SEQUENCE LISTING (76 sequences)

SEQ ID NO: 1 - protein, 12 aa, Artificial Sequence
  EphB1-binding peptide
EWLSPNLAPSVR

SEQ ID NO: 2 - protein, 12 aa, Artificial Sequence
  EphB1-binding peptide
TTLSQLPKSTWL

SEQ ID NO: 3 - protein, 12 aa, Artificial Sequence
  EphB1-binding peptide
AHTFPYPHPKPH

SEQ ID NO: 4 - protein, 12 aa, Artificial Sequence
  EphB1-binding peptide
SHKFPGPPSWMS

SEQ ID NO: 5 - protein, 12 aa, Artificial Sequence
  EphB1-binding peptide
THWKFQPWALVT

SEQ ID NO: 6 - protein, 12 aa, Artificial Sequence
  EphB2-binding peptide
THWCHLLNCAAL

SEQ ID NO: 7 - protein, 12 aa, Artificial Sequence
  EphB2-binding peptide
WHRYPDPRMLPT

SEQ ID NO: 8 - protein, 12 aa, Artificial Sequence
  EphB2-binding peptide
WHWTIEPFAITS

SEQ ID NO: 9 - protein, 12 aa, Artificial Sequence
  EphB2-binding peptide
DHWYYTPWQPIE

SEQ ID NO: 10 - protein, 12 aa, Artificial Sequence
  EphB2-binding peptide
DHWRILPFSLSS

SEQ ID NO: 11 - protein, 12 aa, Artificial Sequence
  EphB2-binding peptide
IHWPVAPYSYLD

SEQ ID NO: 12 - protein, 12 aa, Artificial Sequence
  EphB2-binding peptide
SHWPVLPFAHWQ

SEQ ID NO: 13 - protein, 12 aa, Artificial Sequence
  EphB2-binding peptide
DHWRVSPYSLLY

SEQ ID NO: 14 - protein, 12 aa, Artificial Sequence
  EphB2-binding peptide
SHWPISPYSLLS

SEQ ID NO: 15 - protein, 12 aa, Artificial Sequence
  EphB2-binding peptide
NHWPTQPYAIPI

SEQ ID NO: 16 - protein, 12 aa, Artificial Sequence
  EphB2-binding peptide
DHWPLLPYALAH

SEQ ID NO: 17 - protein, 12 aa, Artificial Sequence
  EphB2-binding peptide
WPPHWPRSLDYA

SEQ ID NO: 18 - protein, 12 aa, Artificial Sequence
  EphB2-binding peptide
SNEWIQPRLPQH

SEQ ID NO: 19 - protein, 12 aa, Artificial Sequence
  EphB2-binding peptide
EWYMKFPPEHYF

SEQ ID NO: 20 - protein, 12 aa, Artificial Sequence
  EphB4-binding peptide
DALNDWLLFRPW

SEQ ID NO: 21 - protein, 12 aa, Artificial Sequence
  EphB4-binding peptide
DHNHNLYNPWRL

SEQ ID NO: 22 - protein, 12 aa, Artificial Sequence
  EphB4-binding peptide
TYFDFQAWSIRA

SEQ ID NO: 23 - protein, 12 aa, Artificial Sequence
  EphB4-binding peptide
EFFTWRPTYYGI

SEQ ID NO: 24 - protein, 12 aa, Artificial Sequence
  EphB4-binding peptide
TNYLFSPNGPIA

SEQ ID NO: 25 - protein, 12 aa, Artificial Sequence
  EphB4-binding peptide
FSPQGPAARNFA

SEQ ID NO: 26 - protein, 11 aa, Artificial Sequence
  EphB4-binding peptide
LPHGPVAAWDA

SEQ ID NO: 27 - protein, 12 aa, Artificial Sequence
  EphB4-binding peptide
NPVIGPIQRAWT

SEQ ID NO: 28 - protein, 12 aa, Artificial Sequence
  EphB4-binding peptide
SHVGPIMRAWAP

SEQ ID NO: 29 - protein, 12 aa, Artificial Sequence
  EphB4-binding peptide
GPVHRAWEPTSH

SEQ ID NO: 30 - protein, 12 aa, Artificial Sequence
  EphB4-binding peptide
GPVERAWRPDLI

SEQ ID NO: 31 - protein, 12 aa, Artificial Sequence
  EphB4-binding peptide
GPVSKAWQETET

SEQ ID NO: 32 - protein, 12 aa, Artificial Sequence
  EphB4-binding peptide
GPVADAWLVYPR

SEQ ID NO: 33 - protein, 12 aa, Artificial Sequence
  EphB4-binding peptide
WGIPRAAQVMWT

SEQ ID NO: 34 - protein, 12 aa, Artificial Sequence
  EphB4-binding peptide
IPWTQHMAMSPM

SEQ ID NO: 35 - protein, 11 aa, Artificial Sequence
  EphB4-binding peptide
SGHQLLNKMPN

SEQ ID NO: 36 - protein, 20 aa, Artificial Sequence
  EphrinB1-binding peptide
KFQEFSPNYMGLEFKKHHDY

SEQ ID NO: 37 - protein, 20 aa, Artificial Sequence
  EphrinB2-binding peptide
KFQEFSPNLWGLEFQKNKDY

SEQ ID NO: 38 - protein, 20 aa, Artificial Sequence
  EphrinB3-binding peptide
KFQEYSPNLWGHEFRSHHDY

SEQ ID NO: 39 - protein, 15 aa, Artificial Sequence
  EphB4-binding peptide
TNYLFSPNGPIARAW

SEQ ID NO: 40 - protein, 14 aa, Artificial Sequence
  EphB4-binding peptide
NYLFSPNGPIARAW

SEQ ID NO: 41 - protein, 13 aa, Artificial Sequence
  EphB4-binding peptide
YLFSPNGPIARAW

SEQ ID NO: 42 - protein, 12 aa, Artificial Sequence
  EphB4-binding peptide
LFSPNGPIARAW

SEQ ID NO: 43 - protein, 12 aa, Artificial Sequence
  DAL peptide
DALNDWLLFRPW

SEQ ID NO: 44 - protein, 12 aa, Artificial Sequence
  IPW peptide
IPWTQHMAMSPM

SEQ ID NO: 45 - protein, 12 aa, Artificial Sequence
  SVS peptide
SVSVGMKPSPRP

SEQ ID NO: 46 - protein, 12 aa, Artificial Sequence
  SGH peptide
SGHQLLLNKMPN

SEQ ID NO: 47 - protein, 12 aa, Artificial Sequence
  EphB4-binding peptide
GPVADAWLVYPR

SEQ ID NO: 48 - protein, 12 aa, Artificial Sequence
  EphB4-binding peptide
NPVIGPIQRAWT

SEQ ID NO: 49 - protein, 12 aa, Artificial Sequence
  EphB4-binding peptide
DHNHDLYNPWRL

SEQ ID NO: 50 - protein, 12 aa, Artificial Sequence
  EphB4-binding peptide
TNYLFSPNGPIA

SEQ ID NO: 51 - protein, 12 aa, Artificial Sequence
  EphB4-binding peptide
LPHGPVAAAWDA

SEQ ID NO: 52 - protein, 12 aa, Artificial Sequence
  EphB4-binding peptide
TYFDFQAWSIRA

SEQ ID NO: 53 - protein, 12 aa, Artificial Sequence
  EphB4-binding peptide
EWYMKFPPEHYF

SEQ ID NO: 54 - protein, 12 aa, Artificial Sequence
  EphB4-binding peptide
GPVHRAWEPTSH

SEQ ID NO: 55 - protein, 12 aa, Artificial Sequence
  EphB4-binding peptide
SHVGPIMRAWAP

SEQ ID NO: 56 - protein, 12 aa, Artificial Sequence
  EphB4-binding peptide
WGIPRAAQVMWT

SEQ ID NO: 57 - protein, 12 aa, Artificial Sequence
  EphB4-binding peptide
GPVSKAWQETET

SEQ ID NO: 58 - protein, 12 aa, Artificial Sequence
  EphB4-binding peptide
EFFTWRPTYYGI

SEQ ID NO: 59 - protein, 12 aa, Artificial Sequence
  EphB4-binding peptide
GPVERAWRPDLI

SEQ ID NO: 60 - protein, 12 aa, Artificial Sequence
  EphB4-binding peptide
DHNHNLYNPWRL

SEQ ID NO: 61 - protein, 12 aa, Artificial Sequence
  EphB4-binding peptide
FSPQGPAARNFA

SEQ ID NO: 62 - protein, 12 aa, Artificial Sequence
  EphB2-binding peptide
SHWPISPYSLLS

SEQ ID NO: 63 - protein, 12 aa, Artificial Sequence
  EphB2-binding peptide
DHWRVSPYSLLY

SEQ ID NO: 64 - protein, 12 aa, Artificial Sequence
  EphB2-binding peptide
SNEWIQPRLPQH

SEQ ID NO: 65 - protein, 12 aa, Artificial Sequence
  EphB2-binding peptide
DHWRILPFSLSS

SEQ ID NO: 66 - protein, 12 aa, Artificial Sequence
  EphB2-binding peptide
SHWPVLPFAHWQ

SEQ ID NO: 67 - protein, 12 aa, Artificial Sequence
  EphB2-binding peptide
IHWPVAPYSYLD

SEQ ID NO: 68 - protein, 12 aa, Artificial Sequence
  EphB2-binding peptide
WHRYPDPRMLPT

SEQ ID NO: 69 - protein, 12 aa, Artificial Sequence
  EphB2-binding peptide
WHWTIEPFAITS

SEQ ID NO: 70 - protein, 12 aa, Artificial Sequence
  EphB2-binding peptide
THWCHLLNCAAL

SEQ ID NO: 71 - protein, 12 aa, Artificial Sequence
  EphB2-binding peptide
DHWYYTPWQPIE

SEQ ID NO: 72 - protein, 12 aa, Artificial Sequence
  EphB2-binding peptide
NHWPTQPYAIPI

SEQ ID NO: 73 - protein, 12 aa, Artificial Sequence
  EphB2-binding peptide
WPPHWPRSLDYA

SEQ ID NO: 74 - protein, 12 aa, Artificial Sequence
  EphB2-binding peptide
DHWPLLPYALAH

SEQ ID NO: 75 - protein, 12 aa, Artificial Sequence
  EphB2-binding peptide
RNKRIRMQLPMI

SEQ ID NO: 76 - protein, 5 aa, Artificial Sequence
  linker sequence
GSGSK

(19) United States
(12) Patent Application Publication
(10) Pub. No.: US 2010/0150834 A1
(43) Pub. Date: Jun. 17, 2010

(54) HIGH AFFINITY EPHB RECEPTOR BINDING COMPOUNDS AND METHODS OF USE THEREOF

(75) Inventor: Elena A. Pasquale, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: THE BURNHAM INSTITUTE, LA JOLLA, CA (US)

(21) Appl. No.: 12/441,274

(22) PCT Filed: Sep. 14, 2007
(86) PCT No.: PCT/US07/20200

§ 371 Date: Mar. 2, 2010

Related U.S. Application Data
(60) Provisional application No. 60/844,851, filed on Sep. 15, 2006.

(51) Int. Cl. A61K 51/08; A61K 38/08; A61K 38/10; A61K 38/16; G01N 33/566; A61P 35/00

(52) U.S. Cl. 424/1.69; 514/15; 514/14; 514/13; 514/12; 436/501

(57) ABSTRACT

The present invention provides peptide-based compounds that selectively bind to a member(s) of the EphB receptor family, including, but not limited to, EphB1, EphB2, EphB3, EphB4, EphB5 and EphB6. In particular, the invention provides multimeric peptides that selectively bind to EphB receptors. The present invention also provides compositions, including pharmaceutical compositions, comprising the EphB receptor binding compounds and a pharmaceutically acceptable carrier or excipient. Methods for identifying compounds that selectively or specifically bind to a member of the EphB receptor family are also provided.

### RELATED APPLICATIONS

This application claims the benefit under 35 U.S.C. §119(e) of U.S. Provisional Application No. 60/844,851 filed Sep. 15, 2006, which is incorporated by reference herein in its entirety.

### GOVERNMENTAL INTERESTS

This invention was made with government support under grant numbers CA116099, CA82713 and HD25938 awarded by the National Institutes of Health and a grant number DAMD17-01-1-0168 awarded by the Department of Defense. The United States Government may have certain rights in this invention.

### 1. FIELD OF THE INVENTION

The present invention provides peptide-based compounds that selectively bind to a member(s) of the EphB receptor family, including, but not limited to, EphB1, EphB2, EphB3, EphB4, EphB5 and EphB6. In particular, the invention provides multimeric peptides that selectively bind to EphB receptors. The present invention also provides compositions, including pharmaceutical compositions, comprising the EphB receptor binding compounds and a pharmaceutically acceptable carrier or excipient. Methods for identifying compounds that selectively or specifically bind to a member of the EphB receptor family are also provided.

### 2. BACKGROUND OF THE INVENTION

The Eph receptors are a large family of receptor tyrosine kinases that regulate a multitude of biological processes in developing, as well as adult tissues by binding a family of ligands called the Ephrins (Murai & Pasquale, 2003, J Cell Sci 116:2823-2832). To date, 14 Eph receptors (EphA1-A8 and EphB1-B6) and 8 Ephrin ligands (EphrinA1-A5 and EphrinB1-B3) have been identified in mammals (see, e.g., “Unified Nomenclature For Eph Family Receptors And Their Ligands, The Ephrins,” by the Eph Nomenclature Committee, reproduced in Cell 90:403-404, 1997; and Zhou et al., 1998, Pharmacol. Ther. 77:151-181). The Ephrin ligands can discriminate between the EphA and EphB classes of receptors. Ephrin-A ligands bind to EphA receptors, with the exception of Ephrin-A5, which at high concentrations can bind to EphB2 (Himanen et al., 2004, Nat Neurosci 7:501-509). Ephrin-B ligands bind to EphB receptors as well as EphA4 (Gale et al., 1996, Neuron 17:9-19; Kullander & Klein, 2002, Nat Rev Mol Cell Biol 3:475-486). However, interactions between Eph receptors and Ephrins belonging to the same class are highly promiscuous (Murai & Pasquale, 2003, J Cell Sci 116:2823-2832; Kullander & Klein, 2002, Nat Rev Mol Cell Biol 3:475-486; Flanagan & Vanderhaeghen, 1998, Annu Rev Neurosci 21:309-345). In general, binding of Eph receptors by their ligands induce receptor clustering, transphosphorylation, and downstream signaling. With certain Eph receptors, such as EphA2, tyrosine phosphorylation of caused by agonists mediates receptor autophosphorylation, internalization of the receptor and the agonist and subsequent receptor degradation (Zantek, N. D. et al. 1999 Cell Growth Differ 10:629-638; Carles-Kinch, K. et al. 2002 Cancer Res 62:2840-2847; Van der Geer, P. et al. 1994 Annu Rev Cell Biol 10:251-337).

The Eph receptors are differentially expressed in a variety of healthy tissues (Hafner et al., 2004, Clinical Chem 50:490-499) and have been implicated in a variety of aspects of normal functions, such as pain processing (Battaglia et al., 2003, Nat Neurosci 6:339-340), platelet aggregation, embryogenesis (Holder & Klein, 1999, Devel. 126:2033-2044), neuronal development, cell migration and adhesion (Prevost et al., 2005, PNAS USA 102:9820-9825). Furthermore, these receptors have been reported to play a role in the balance of stem cell self-renewal versus cell-fate determination and differentiation (Conover et al., 2000, Nat Neurosci 3:1091-1097; Aoki et al., 2004, J Biol Chem 279:32643-32650; Moore et al., 2004, Dev Cell 6:55-67). The Eph receptors have also been implicated in a variety of pathological processes, including tumor progression (Dodelet & Pasquale, 2000, Oncogene 19:5614-5619; Nakamoto & Bergemann, 2002, Microsc Res Tech 59:58-67; Walker-Daniels et al., 2003, Am J Pathol 162:1037-1042; Hu et al., 2005, Cancer Res 65:2542-2546; Hafner et al., 2004, Clin Chem 50:490-499), pathological forms of angiogenesis (Adams & Klein, 2000, Trends Cardiov Medicine 10:183-188; Brantley-Sieders & Chen, 2004, Angiogenesis 7:17-28; Noren et al., 2004, PNAS USA 101:5583-5588; Cheng et al., 2002, Cytokine & Growth Factor Rev. 13:75-85), chronic pain following tissue damage (Battaglia et al., 2003, Nat Neurosci 6:339-340), inhibition of nerve regeneration after spinal cord injury (Goldshmit et al., 2004, J Neurosci 24:10064-10073), and human congenital malformations (Twigg et al., 2004, PNAS USA 101:8652-8657; Wieland et al., 2004, Am J Hum Genet 74:1209-1215).

Specific Eph receptors have been implicated in various disease processes, including cancer. For example, EphA2 has been shown to be a powerful oncoprotein sufficient to confer malignant transformation and tumorigenic potential on non-transformed mammary epithelial cells when overexpressed, and is upregulated in many aggressive cancer cells such as prostate cancer and breast cancer (Walker-Daniels, 1999, Prostate 41:275-280; Zelinski et al., 2001, Cancer Res. 61:2301-2306; Carles-Kinch et al., 2002, Cancer Res. 62:2840-2847). With respect to the EphB receptor family, EphB4, which is transcribed at high levels in the adult liver, lung, kidney, intestine, placenta, muscle and heart tissues (Zhou et al., 1998, Pharmacol. Ther. 77:151-181), has been shown to be upregulated in colon cancer (Stephenson et al., 2001, BMC Mol. Biol. 2:15), breast cancer (Wu et al., 2004, Pathology Oncology Res. 10:26-33) as well as prostate carcinoma (Lee et al., 2005, BMC Cancer 5:119). In addition, EphB1, EphB2, EphB3, EphB4 and EphB6 have all been shown to be expressed at high levels in several small cell lung carcinoma lines (Tang et al., 1999, Clin. Cancer Res. 5:455-460). Thus, the Eph receptors may represent useful targets for cancer therapies.

### 3. SUMMARY OF THE INVENTION

The present invention provides compounds which selectively bind to a member of the EphB receptor family, including, but not limited to, EphB1, EphB2, EphB3, EphB4, EphB5 and EphB6. In specific embodiments, the invention provides multimeric peptides that selectively bind a member of the EphB receptor family and compete with and/or inhibit binding of an Ephrin-B ligand (e.g., Ephrin-B1, Ephrin-B2 and Ephrin-B3) to the EphB receptor, wherein the multimeric peptides comprise two or more EphB receptor binding peptides. In more specific embodiments, the invention provides multimeric peptides comprising two or more EphB receptor binding peptides identified by SEQ ID NOS:1-75, or disclosed in Table 1, infra. The multimeric peptides can comprise two or more EphB receptor binding peptides that are the same, or can comprise at least one or more different EphB receptor binding peptides (identified by SEQ ID NOS:1-75, or disclosed in Table 1, infra). In a specific embodiment, the multimeric peptides comprise at least two EphB receptor binding peptides (e.g., are dimers) having the amino acid sequence of SEQ ID NO:39. In another specific embodiment, a multimeric peptide comprises at least two EphB receptor binding peptides (e.g., are dimers) having the amino acid sequence of SEQ ID NO:41.

In other embodiments, the invention provides conjugates comprising one or more EphB receptor binding peptides (preferably, two or more) and a heterologous compound, wherein the conjugates compete with and/or inhibit binding of an EphrinB ligand (e.g., Ephrin-B1, Ephrin-B2 and Ephrin-B3) to an EphB receptor. In specific embodiments, the invention provides conjugates comprising one or more EphB receptor binding peptides (in a specific embodiment, two or more) identified by SEQ ID NOS:1-75 or disclosed in Table 1, infra, and a heterologous compound, wherein the conjugates compete with and/or inhibit binding of an EphrinB ligand (e.g., Ephrin-B1, Ephrin-B2 and Ephrin-B3) to an EphB receptor. In certain embodiments, the heterologous compound is polyethylene glycol (“PEG”), an Fc region of an IgG immunoglobulin (e.g., human IgG1 immunoglobulin) or a fragment (e.g., CH2 or CH3 domain) thereof, or other therapeutic or diagnostic agents. The conjugates can comprise two or more EphB receptor binding peptides that are the same, or can comprise at least two or more different EphB receptor binding peptides in which at least one peptide differs from the others (e.g., EphB receptor binding peptides identified by SEQ ID NOS:1-75, or disclosed in Table 1, infra). In a specific embodiment, a conjugate is a fusion protein. In a specific embodiment, a heterologous compound is not a therapeutic, diagnostic, cytotoxic agent or marker. In another specific embodiment, a conjugate comprising heterologous compound is not produced by peptide pegylation, glycosylation, acetylation, formylation, amidation, phosphorylation or other similar chemical modifications of an EphB receptor binding peptide. In another specific embodiment, a heterologous compound is not biotin.

In a specific embodiment, a conjugate comprises one or more EphB receptor binding peptides identified by SEQ ID NOS:1-75 or disclosed in Table 1, infra, and an Fc region of the human IgG1 immunoglobulin or a fragment (e.g., CH2 or CH3 domain) thereof. In another specific embodiment, a conjugate comprises an EphB receptor binding peptide having the amino acid sequence of SEQ ID NO:39 and an Fc region of the human IgG1 immunoglobulin or a fragment (e.g., CH2 or CH3 domain) thereof. In another specific embodiment, a conjugate comprises an EphB receptor binding peptide having the amino acid sequence of SEQ ID NO:41 and an Fc region of the human IgG1 immunoglobulin or a fragment (e.g., CH2 or CH3 domain) thereof. In certain embodiments, the one or more EphB receptor binding peptides is linked to a heterologous compound using techniques known in the art for conjugate synthesis, i.e., using recombinant/genetic engineering, or by chemical methods. Examples of such methods are disclosed in Section 5.1, infra. Various chemical and peptide linkers that are used to generate the EphB binding conjugates are also disclosed in Section 5.1, infra.

In some embodiments, the EphB receptor binding compounds are agonistic (e.g., they induce Eph receptor clustering, transphosphorylation, downstream signaling, EphB receptor internalization and/or degradation).

In other embodiments, the EphB receptor binding compounds are antagonistic (e.g., they inhibit EphB receptor clustering, transphosphorylation, downstream signaling, EphB receptor internalization and/or degradation).

The present invention further provides methods of identifying and producing EphB receptor binding peptides and EphB receptor binding compounds. Methods of identifying EphB receptor binding peptides and EphB receptor binding compounds include, but are not limited to, high through-put screening assays and the like. Methods of generating or producing the EphB receptor binding peptides and EphB receptor binding compounds include, but are not limited to, recombinant/genetic engineering methods and/or chemical synthesis. See Section 5.1, infra, for a more detailed description of such methods.

In specific embodiments, the invention further provides derivatives of the EphB receptor binding peptides and EphB receptor binding compounds, wherein the sequence of the EphB receptor binding peptides (such as those listed in Table 1) or EphB receptor binding compounds have been altered by the introduction of amino acid residue deletions, additions and/or substitutions. In specific embodiments, the derivatives may be more stable (e.g., more resistant to proteolysis) and have increased binding affinity for one or more EphB receptors. Methods for generating or producing derivatives of the EphB receptor binding peptides or EphB receptor binding compounds are known to one of skill in the art and are discussed in Section 5.1, infra.

In a specific embodiment, an EphB receptor binding compound selectively binds to an EphB receptor with a kₒₙ rate of at least 10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹.

In another embodiment, an EphB receptor binding compound selectively binds to an EphB receptor with a k_{off} rate of 5×10⁻¹ s⁻¹ or less, 10⁴ s⁻¹ or less, 5×10⁻² s⁻¹ or less, 10⁻² s⁻¹ or less, 5×10⁻³ s⁻¹ or less, 10⁻³ s⁻¹ or less, 5×10⁻⁴ s⁻¹ or less, 10⁻⁴ s⁻¹ or less, 5×10⁻⁵ s⁻¹ or less, 10⁻⁵ s⁻¹ or less, 5×10⁻⁶ s⁻¹ or less, 10⁻⁶ s⁻¹ or less, 5×10⁻⁷ s⁻¹ or less, 10⁻⁷ s⁻¹ or less, 5×10⁴ s⁻¹ or less, 10⁻⁸ s⁻¹ or less, 5×10⁻⁹ s⁻¹ or less, 10⁻⁹ s⁻¹ or less, 5×10⁻¹⁰ s⁻¹ or less, or 10⁻¹⁰ s⁻¹ or less.

In another embodiment, an EphB receptor binding compound selectively binds to an EphB receptor with a Kₐ (kₒₙ/k_{off}) of at least 10¹¹ nM⁻¹, at least 5×10¹¹ nM⁻¹, at least 10¹² nM⁻¹, at least 5×10¹² nM⁻¹, at least 10¹³ nM⁻¹, at least 5×10¹³ nM⁻¹, at least 10¹⁴ nM⁻¹, at least 5×10¹⁴ nM⁻¹, at least 10¹⁵ nM⁻¹, at least 5×10¹⁵ nM⁻¹, at least 10¹⁶ nM⁻¹, at least 5×10¹⁶ nM⁻¹, at least 10¹⁷ nM⁻¹, at least 5×10¹⁷ nM⁻¹, at least 10¹⁸ nM⁻¹, at least 5×10¹⁸ nM⁻¹, at least 10¹⁹ nM⁻¹, at least 5×10¹⁹ nM⁻¹, at least 10²⁰ nM⁻¹, at least 5×10²⁰ nM⁻¹, at least 10²¹ nM⁻¹, at least 5×10²¹ nM⁻¹, at least 10²² at least 5×10²² nM⁻¹, at least 10²³ nM⁻¹, at least 5×10²³ nM⁻¹, at least 10²⁴ nM⁻¹, or at least 5×10²⁴ nM⁻¹.

In yet another embodiment, an EphB receptor binding compound selectively binds to an EphB receptor with a K_{d} (k_{off}/kₒₙ) of 5×10⁷ nM or less, 10⁷ nM or less, 5×10⁶ nM or less, 10⁶ nM or less, 5×10⁵ nM or less, 10⁵ nM or less, 5×10⁴ nM or less, 10⁴ nM or less, 5×10³ nM or less, 10³ nM or less, 5×10² nM or less, 100 nM or less, 90 nM or less, 80 nM or less, 70 nM or less, 60 nM or less, 50 nM or less, 10 nM or less, 5 nM or less, 3.8 nM or less, 1 nM or less, 5×10⁻¹ nM or less, 10⁻¹ nM or less, 5×10⁻² nM or less, 10⁻² nM or less, 5×10⁻³ nM or less, 10⁻³ nM or less, 5×10⁻⁴ nM or less, 10⁻⁴ nM or less, 5×10⁻⁵ nM or less, 10⁻⁵ nM or less, 5×10⁻⁶ nM or less, or 10⁻⁶ nM or less.

In one embodiment, an EphB receptor binding compound binds to an EphB receptor and has an IC₅₀ value of less than 5×10⁷ nM, less than 10⁷ nM, less than 5×10⁶ nM, less than 10⁶ nM, less than 5×10⁵ nM, less than 10⁵ nM, less than 5×10⁴ nM, less than 10⁴ nM, less than 5×10³ nM, less than 10³ nM, less than 5×10² nM, less than 100 nM, less than 90 nM, less than 80 nM, less than 70 nM, 69 nM, less than 60 nM, less than 50 nM, less than 40 nM, less than 30 nM, less than 20 nM, 12 nM, less than 5 nm, less than 1 nM, less than 5×10⁻¹ nM, less than 10⁻¹ nM, less than 5×10⁻² nM, less than 10⁻² nM, less than 5×10⁻³ nM, less than 10⁻³ nM, less than 5×10⁻⁴ nM, or less than 10⁻⁴ nM when measured according to methods well known in the art or described herein, e.g., ELISA. In other embodiments, an EphB receptor binding peptide binds to EphB4 and has an approximate IC₅₀ value of between approximately 1 nM and approximately 10 nM, between approximately 10 nM and approximately 10² nM, between approximately 10² nM and approximately 10³ nM, between approximately 10³ nM and approximately 10⁴ nM, between approximately 10⁴ nM and approximately 10⁵ nM, between approximately 10⁵ nM and approximately 10⁶ nM, or between approximately 10⁶ nM and approximately 10⁷ nM when measured according to methods well known in the art or described herein, e.g., ELISA. In particular embodiments, an EphB receptor binding compound binds to EphB4 and has an IC₅₀ value of approximately 0.1, 0.5, 1, 5, 10, 20, 30, 40, 50, 60, 70, 80, 90, 100, 110, 120, 130, 140 or 150 nM. In specific embodiments, an EphB receptor binding peptide binds to EphB4 and has an IC₅₀ value of approximately 12 nM or approximately 16 nM. In particular embodiments, the EphB receptor binding compounds bind to murine EphB4. In other embodiments, the EphB receptor binding compounds bind to human EphB4. In certain embodiments, an EphB receptor binding peptide selectively binds to EphB4 and inhibits the binding of the EphB4 receptor to the Ephrin B2 ligand at an IC₅₀ value of approximately 69 nM, approximately 70 nM, approximately 75 nM, or approximately 80 nM.

The present invention further provides compositions and prophylactic and therapeutic regimens designed to prevent, treat, and/or manage an EphB receptor related disease, including, but not limited to, neoplastic disease, cancer, vascular disease (e.g., macular degeneration), neurological disease, pathological forms of angiogenesis, chronic pain following tissue damage, inhibition of nerve regeneration after spinal cord injury and human congenital malformations. In specific embodiments, an EphB receptor related disease comprises cells that overexpress one or more members of the EphB receptor family, as in, for example, cancer. Cancers to be prevented, treated and/or managed using the methods presented herein include cancers of an epithelial or endothelial cell origin. Non-limiting examples of such cancers include mesothelioma, ovarian cancer, bladder cancer, squamous cell carcinoma of the head and neck, breast cancer, prostate cancer, colon cancer, small cell lung carcinoma and cancers of neurological origin.

In a specific embodiment, a composition comprises one or more EphB receptor binding compounds (e.g., a multimeric peptide and/or a conjugate described herein) and a pharmaceutically acceptable carrier or excipient. Such compositions can further comprise additional therapies, such as chemotherapies, hormonal therapies and/or biological therapies, immunotherapies, and can be administered in conjunction with radiation therapies and/or surgery. Thus, the EphB receptor binding compounds (e.g., multimeric peptides and/or conjugates described herein) can be administered in combination with a therapeutically or prophylactically effective amount of one or more additional therapies as described above. The one or more additional therapies can be administered concurrently, before or after administration of the EphB receptor binding compounds (e.g., multimeric peptides and/or conjugates described herein).

The methods and compositions are useful not only in untreated patients but are also useful in the treatment of patients that are partially or completely refractory to current standard and experimental therapies for EphB receptor related diseases, including but not limited to, chemotherapies, hormonal therapies, biological therapies, immunotherapies, radiation therapies, and/or surgery. Accordingly, in some embodiments, the invention provides therapeutic and prophylactic methods for the treatment, prevention and/or management of EphB receptor related diseases that have been shown to be or may be refractory or non-responsive to therapies other than those comprising administration of the EphB receptor binding compounds (e.g., multimeric peptides and/or a conjugate described herein). In a specific embodiment, one or more EphB receptor binding compounds (e.g., multimeric peptides and/or a conjugate described herein) are administered to a patient refractory or non-responsive to a non-EphB receptor binding compound-based therapy to render the patient non-refractory or responsive. The therapy to which the patient had previously been refractory or non-responsive can then be administered with therapeutic effect.

The invention provides for methods of preventing, treating or managing an EphB receptor related disease comprising administering to a subject in need thereof an effective amount of an isolated EphB receptor binding compound. In specific embodiments, the invention provides for methods of treating cancer comprising administering to a subject in need thereof a therapeutically effective amount of an isolated EphB receptor binding compound following removal or a tumor from the subject. In other embodiments, the invention provides for methods of preventing or treating a relaspe of cancer comprising administering to a subject in need thereof a prophylactically or therapeutically effective amount of an isolated EphB receptor binding compound. In particular embodiments, the invention provides for methods of reducing the number of EphB receptor expressing cells (e.g., cells that aberrantly express an EphB recepter) and methods of inhibiting proliferation of EphB receptor expressing cells (e.g., cells that aberrantly express an EphB recepter), said methods comprising contacting the cells with an effective amount of an isolated EphB receptor binding compound. The invention also provides for methods of reducing the size of a tumor and methods of preventing growth of a tumor, said methods comprising contacting the tumor with an effective amount of an isolated EphB receptor binding compound.

The invention further provides methods of diagnosing, prognosing, or monitoring an EphB related disease in a subject, using the EphB receptor binding compounds (e.g., multimeric peptides and/or conjugates described herein) to evaluate the efficacy of a treatment for an EphB receptor related disease, either EphB receptor binding compound-based or non EphB receptor binding compounds-based. For example, but not by way of limitation, increased EphB2 or EphB4 expression may be associated with increasingly invasive and metastatic cancers. Accordingly, a reduction in EphB2 or EphB4 expression with a particular treatment indicates that the treatment is reducing the invasiveness and/or metastatic potential of a cancer associated with EphB2 or EphB4. Other determinants or endpoints to measure efficacy of a particular EphB receptor binding compound (e.g., multimeric peptides and/or conjugates described herein) include cell proliferation, EphB receptor transphosphorylation, EphB receptor clustering and EphB receptor degradation. In the context of cancer, to test whether an agonistic EphB receptor binding compound (e.g., a multimeric peptide or conjugates thereof) is efficacious, the EphB receptor binding compound is contacted with cells from a patient, e.g., cancer cells, and assays are performed to determine the effect of the EphB receptor binding compound on various endpoints, such as proliferation of the cancer cells, EphB receptor clustering, transphosphorylation, internalization and/or degradation. In a specific embodiment, an agonistic EphB receptor binding compound is determined to be efficacious if: (1) there is a decrease or inhibition of proliferation of the cancer cells relative to a control; (2) there is an increase in EphB receptor transphosphorylation relative to a control; (3) there is an increase in EphB receptor clustering relative to a control; (4) there is an increase in EphB receptor internalization relative to a control; (5) there is an increase in EphB receptor degradation relative to a control; (6) there is an increase in apoptosis relative to a control; or (7) there is a decrease or inhibition of cell migration/invasion relative to a control. In another specific embodiment, an antagonistic EphB receptor binding compound (e.g., a multimeric peptide) is determined to be efficacious if: (1) there is an decrease of proliferation of the cancer cells relative to a control; (2) there is decrease in EphB receptor transphosphorylation relative to a control; (3) there is a decrease in EphB receptor clustering relative to a control; (4) there is decrease in EphB receptor internalization relative to a control; or (5) there is decrease in EphB receptor degradation relative to a control. Methods for measuring such endpoints are known to one of skill in the art, and are described further in Section 5.3, infra.

Presented herein are methods for the detection and diagnosis of EphB receptor related diseases utilizing EphB receptor binding compounds. In particular embodiments, detection and diagnostic methods presented herein provide methods of imaging and localizing metastases and methods of diagnosis and prognosis using tissues and fluids distal to the primary tumor site (as well as methods using tissues and fluids of the primary tumor), for example, whole blood, sputum, urine, serum, fine needle aspirates (i.e., biopsies). In other embodiments, the diagnostic methods presented herein provide methods of imaging and localizing metastases and methods of diagnosis and prognosis in vivo. In such embodiments, primary metastatic tumors are detected using an EphB receptor binding compound (e.g., a multimeric peptide and/or a conjugate described herein). The EphB receptor binding compounds may also be used for immunohistochemical analyses of frozen or fixed cells or tissue assays.

The present invention further provides kits comprising an EphB receptor binding compound alone or in combination with other therapeutic or diagnostic reagents.

### 3.1 Definitions

As used herein, the terms “about” and “approximately,” when used to a modify numeric value or numeric range, indicate that reasonable deviations from the value or range, typically 10% above and 10% below the value or range, remain within the intended meaning of the recited value or range.

As used herein, the term “agent” refers to a molecule that has a desired biological effect. An agent can be prophylactic, therapeutic or diagnostic. Agents include, but are not limited to, proteinaceous molecules, including, but not limited to, peptides (including dimers and multimers of such peptides), polypeptides, proteins, including post-translationally modified proteins, conjugates, antibodies, etc.; small molecules (less than 1000 daltons), including inorganic or organic compounds; nucleic acid molecules including, but not limited to, double-stranded or single-stranded DNA, or double-stranded or single-stranded RNA (e.g., antisense, RNAi, etc.), intron sequences, triple helix nucleic acid molecules and aptamers; or vaccines. Agents can be derived from any known organism (including, but not limited to, animals, plants, bacteria, fungi, and protista, or viruses) or from a library of synthetic molecules.

As used herein, “agonist” or “agonistic” refers to an agent (e.g., an EphB receptor binding peptide or an EphB receptor binding compound) that selectively binds to a member(s) of the EphB family of receptors (e.g., EphB1, EphB2, EphB3, EphB4, EphB5 or EphB6) and elicits signaling of the EphB receptor (e.g., it causes EphB receptor clustering, transphosphorylation and/or activation of downstream signaling pathways).

As used herein, “antagonist” or “antagonistic” refers to an agent that selectively binds to a member(s) of the EphB family of receptors (e.g., EphB1, EphB2, EphB3, EphB4, EphB5 or EphB6) and inhibits or reduces signaling of the EphB receptor (e.g., it inhibits or reduces EphB receptor clustering, transphosphorylation and/or activation of downstream signaling pathways).

As used herein, the term “analog” in the context of a peptide that selectively or specifically binds to a member(s) of the EphB family of receptors (e.g., EphB1, EphB2, EphB3, EphB4, EphB5 or EphB6), and in particular, a peptide disclosed in Table 1, infra, refers to a peptide that possesses a similar or identical function as a second peptide but does not necessarily comprise a similar or identical amino acid sequence or structure of the second peptide. A peptide that has a similar amino acid sequence refers to a first peptide that satisfies at least one of the following: (a) a first peptide having an amino acid sequence that is at least 70%, at least 75%, at least 80%, at least 85%, at least 90%, at least 95%, or at least 99% identical to the amino acid sequence of a second peptide; (b) a first peptide encoded by a nucleotide sequence that hybridizes under stringent conditions to a nucleotide sequence encoding a second peptide of 100 amino acid residues or less, 90 amino acid residues or less, 80 amino acid residues or less, 70 amino acid residues or less, 60 amino acid residues or less, 50 amino acid residues or less, 40 amino acid residues or less, 30 amino acid residues or less, 20 amino acid residues or less, 12 amino acid residues or less, 10 amino acid residues or less, 8 amino acid residues or less with a minimum of 4 or 5 amino acid residues; (c) to a first peptide encoded by a nucleotide sequence that is at least 60%, at least 65%, at least 70%, at least 75%, at least 80%, at least 85%, at least 90%, at least 95% or at least 99% identical to the nucleotide sequence encoding a second peptide. In a specific embodiment, an analog of an EphB receptor binding peptide is a peptidomimetic.

A peptide with similar structure to a second peptide refers to peptide that has similar secondary, tertiary or quaternary structure of the peptide. The structure of a peptide can be determined by methods known to those skilled in the art, including but not limited to, X-ray crystallography, nuclear magnetic resonance, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A non-limiting example of a mathematical algorithm utilized for the comparison of two sequences is the algorithm of Karlin and Altschul, 1990, Proc. Natl. Acad. Sci. U.S.A. 87: 2264-2268, modified as in Karlin and Altschul, 1993, Proc. Natl. Acad. Sci. U.S.A. 90: 5873-5877. Such an algorithm is incorporated into the NBLAST and XBLAST programs of Altschul et al., 1990, J. Mol. Biol. 215: 403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25: 3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another non-limiting example of a mathematical algorithm utilized for the comparison of sequences is the algorithm of Myers and Miller, 1988, CABIOS 4: 11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of an organic or inorganic molecule other than a peptide that selectively binds to a member(s) of the EphB family of receptors (e.g., EphB1, EphB2, EphB3, EphB4, EphB5 or EphB6) refers to a second organic or inorganic molecule which possesses a similar or identical function as a first organic or inorganic molecule and is structurally similar to the first organic or inorganic molecule.

As used herein, the term “cancer” refers to a neoplasm or tumor resulting from abnormal uncontrolled growth of cells. Non-limiting examples include those cancers described in Section 5.2.2.1, infra. The term “cancer” encompasses a disease involving both pre-malignant and malignant cancer cells. In some embodiments, cancer refers to a localized overgrowth of cells that has not spread to other parts of a subject, i.e., a benign tumor. In other embodiments, “cancer” refers to a disease involving cells that have the potential to metastasize to distal sites and exhibit phenotypic traits that differ from those of non-cancer cells, for example, formation of colonies in a three-dimensional substrate such as soft agar or the formation of tubular networks or web-like matrices in a three-dimensional basement membrane or extracellular matrix preparation, such as MATRIGEL™. Such non-cancer cells do not form colonies in soft agar and can form distinct hollow sphere-like structures in three-dimensional basement membrane or extracellular matrix preparations. The term “cancer cell” is meant to encompass both pre-malignant and malignant cancer cells.

As used herein, the term “conjugate” in the context of an EphB receptor binding compound refers to an agent that selectively or specifically binds to a member(s) of the EphB receptor family (e.g., EphB1, EphB2, EphB3, EphB4, EphB5 or EphB6), comprising two or more EphB receptor binding peptides (e.g., those disclosed in Table 1, infra), or one or more EphB receptor binding peptides and a heterologous compound. Thus, the term “heterologous compound” in the context of a conjugate refers to any compound that is different in amino acid sequence and/or structure than the EphB receptor binding peptide to which it is conjugated. The components of a conjugate may be directly fused, using either non-covalent bonds or covalent bonds (e.g., by combining amino acid sequences via peptide bonds), and/or may be combined using one or more linkers. Linkers suitable for preparing conjugates comprise peptides, alkyl groups, chemically substituted alkyl groups, polymers, or any other covalently-bonded or non-covalently bonded chemical substance capable of binding together two or more components resulting in an EphB receptor binding compound. Polymer linkers comprise any polymers known in the art, including polyethylene glycol (“PEG”). By using methods of chemical synthesis known in the art and/or described herein, a conjugate can be prepared by coupling PEG to one or more EphB receptor binding peptides (as well as analogs or derivatives thereof), thereby providing an EphB receptor binding compound. In a specific embodiment, a conjugate is a fusion protein that selectively or specifically binds to a member of the EphB receptor family (e.g., EphB1, EphB2, EphB3, EphB4, EphB5, or EphB6), comprising two proteinaceous molecules that are linked together via a peptide bond.

As used herein, the term “derivative” in the context of a peptide that selectively or specifically binds to a member of the EphB receptor family (e.g., EphB1, EphB2, EphB3, EphB4, EphB5, or EphB6) refers to a peptide that comprises the amino acid sequence which has been altered by the introduction of amino acid residue deletions, additions and/or substitutions. In a specific embodiment, a derivative in the context of an EphB receptor binding peptide refers to an EphB receptor binding compound composed of an amino acid sequence identical to the amino acid sequence of a second EphB receptor binding compound except for 1-8, 1-6, 1-4, 1-3, 1, 2, 3, 4, 5, 6, 7, or 8 amino acid deletions. In another embodiment, the term derivative refers to an EphB receptor binding compound composed of an amino acid sequence identical to the amino acid sequence of a second EphB receptor binding compound except for 1-5, 1-4, 1-3, 1, 2, 3, 4 or 5 amino acid residue substitutions. In another embodiment, the term derivative refers to an EphB receptor binding compound composed of an amino acid sequence identical to a second EphB receptor binding compound, except for the addition of 1-12, 1-10, 1-8, 1-6, 1-4, 1, 2, 3, 4, 5, 6, 7, 8, 9, 10, 11 or 12 amino acid residues at the amino or carboxy terminus of an EphB receptor binding compound. In yet another embodiment, the term derivative in the context of an EphB receptor binding compound refers to an EphB receptor binding compound composed of an amino acid sequence identical to the amino acid sequence of a second EphB receptor except for a combination of 1-8, 1-6, 1-4, 1-3, 1, 2, 3, 4, 5, 6, 7, or 8 amino acid residue deletions and substitutions, amino acid residue deletions and additions, amino acid residue substitutions and additions, or amino acid residue deletions, substitutions and additions.

A derivative of an EphB receptor binding peptide may also be produced by chemical modifications using techniques known to those of skill in the art, including, but not limited to specific chemical cleavage, acetylation, formylation, metabolic synthesis of tunicamycin, etc. Further, a derivative of an EphB receptor binding peptide may contain one or more non-classical amino acids.

A derivative of a peptide that selectively or specifically binds to a member of the EphB family of receptors (e.g., EphB1, EphB2, EphB3, EphB4, EphB5 or EphB6) possesses an identical function(s) as the EphB receptor binding peptide from which it was derived. In one embodiment, a derivative of an EphB receptor binding compound possesses a similar or identical function as an Ephrin-B ligand. In another embodiment, an EphB receptor binding peptide derivative has improved activity when compared to the EphB receptor binding peptide from which it was derived. For example, an EphB receptor binding peptide derivative can bind to an EphB receptor more tightly and/or is more resistant to proteolysis than the EphB receptor binding peptide from which it was derived.

As used herein, “detectable label” or “imaging agent” refers to materials, which when covalently attached to a compound, permit detection of the compound, including but not limited to, detection in vivo in a patient to whom an agent that selectively or specifically binds to an Eph receptor binding agent (e.g., an EphB receptor binding compound) has been administered. Suitable detectable labels are well known in the art and include, by way of example, biotin, alkaline phosphatase, radioisotopes, fluorescent labels (for example, fluorescein), and the like. The particular detectable label employed is not critical and is selected relative to the amount of label to be employed as well as the toxicity of the label at the amount of label employed. Selection of the label relative to such factors is well within the skill of the art.

Covalent attachment of the detectable label to the peptide or peptidomimetic is accomplished by conventional methods well known in the art. For example, when the ¹²⁵I radioisotope is employed as the detectable label, covalent attachment of ¹²⁵I to the peptide, peptidomimetic or multimers thereof can be achieved by incorporating the amino acid tyrosine into the peptide or peptidomimetic and then iodinating the peptide (see, for example, Weaner, et al. 1994 Synthesis and Applications of Isotopically Labelled Compounds, pp. 137-140). If tyrosine is not present in the peptide or peptidomimetic, incorporation of tyrosine to the amino or carboxy terminus of the peptide or peptidomimetic can be achieved by well known chemistry. Likewise, ³²P can be incorporated onto the peptide or peptidomimetic as a phosphate moiety through, for example, a hydroxyl group on the peptide or peptidomimetic using conventional chemistry.

The terms “disease” and “disorder” can be used interchangeably to refer to a condition, in particular, a pathological condition, and more particularly an EphB related disease.

As used herein, the term “effective amount” in the context of a prophylactic and/or therapeutic utility refers to the amount of a therapy (e.g., a prophylactic or therapeutic agent) which has a beneficial prophylactic or therapeutic effect in a subject. In a specific embodiment, an effective amount is the amount of therapy sufficient to result in one, two, three or more of the following: (1) reduce and/or ameliorate the severity of a disease or a symptom thereof; (2) reduce the duration of a disease or a symptom thereof; (3) prevent the advancement of a disease; (4) cause regression of said disease; (5) prevent the recurrence, development, or onset of a disease or a symptom thereof, (6) reduce the number of symptoms of a disease; (7) decrease length of hospitalization; (8) decrease relapse; (9) decrease spread of disease to different cells, tissues, and/or organs; (10) decrease mortality; (11) increase survival; (12) decrease the size of a tumor; (13) inhibit or reduce the proliferation of EphB receptor expressing cells (e.g., cells that aberrantly express an EphB receptor); (14) reduce the number of EphB receptor expressing cells (e.g., cells that aberrantly express an EphB receptor); and/or (15) enhance or improve the prophylactic or therapeutic effect(s) of another therapy (e.g., prophylactic or therapeutic agent). Non-limiting examples of effective amounts of EphB receptor binding compounds are provided in Section 5.4.3, infra.

As used herein, the term “endogenous ligand” or “natural ligand” refers to a molecule that normally binds a particular receptor in vivo. For example, and not by way of limitation, any of the A-type Ephrin ligands (e.g., EphrinA1, EphrinA2, EphrinA3, EphrinA4 and EphrinA5) may bind to any of the A-type Eph receptors (e.g., EphA1, EphA2, EphA3, EphA4, EphA5, EphA6, EphA7 and EphA8); and any of the B-type Ephrin ligands (e.g., EphrinB1, EphrinB2 and EphrinB3) may bind to any of the B-type Eph receptors (e.g., EphB1, EphB2, EphB3, EphB4, EphB5 and EphB6). Also, by way of example and not by way of limitation, EphA4 may bind to both A-type and B-type Ephrin ligands as disclosed herein.

As used herein, the term “Eph receptor” or “Eph receptor tyrosine kinase” refers to any Eph receptor that has or will be identified and recognized by one of skill in the art (see, e.g., Eph Nomenclature Committee, 1997, Cell 90:403-404). EphB receptors include, but are not limited to EphB1, EphB2, EphB3, EphB4, EphB5 and EphB6. In a specific embodiment, an EphB receptor is from any species. In a specific embodiment, an EphB receptor is human. The nucleotide and/or amino acid sequences of Eph receptors can be found in the literature or public databases (e.g., GenBank), or the nucleotide and/or amino acid sequences can be determined using cloning and sequencing techniques known to one of skill in the art. Non-limiting examples of GenBank Accession numbers for amino acid sequences of EphB receptors include P54762 (human EphB1); NP_{—}059145 (human EphB2); NP_{—}004434 (human EphB3); A54092 (human EphB4); and NP_{—}004436 (human EphB6).

As used herein, the term “Ephrin” or “Ephrin ligand” refers to any Ephrin ligand that has or will be identified and recognized by one of skill in the art (see, e.g., Eph Nomenclature Committee, 1997, Cell 90:403-404). As used herein, “Ephrin-B” includes any of the Ephrins that are members of the Ephrin-B ligand subclass. Ephrins of the present invention include, but are not limited to, Ephrin-B1, Ephrin-B2 and Ephrin-B3. In a specific embodiment, an Ephrin is from any species. In a specific embodiment, an Ephrin is human. The nucleotide and/or amino acid sequences of Ephrins can be found in the literature or public databases (e.g., GenBank), or the nucleotide and/or amino acid sequences can be determined using cloning and sequencing techniques known to one of skill in the art. Non-limiting examples of GenBank Accession numbers for amino acid sequences of Ephrin-B ligands include 546993 (human Ephrin B1); 2108400B (human Ephrin B2); and NP_{—}501955 (Ephrin B3).

As used herein, the term “EphB receptor binding peptide(s)” refers to a peptide that selectively or specifically binds to a member of the EphB family of receptors (e.g., EphB1, EphB2, EphB3, EphB4, EphB5 or EphB6), and includes analogs and derivatives thereof. In specific embodiments, an EphB receptor binding peptide comprises a minimum of 4 or 5 amino acid residues and a maximum of 50 amino acid residues. Examples of EphB receptor binding peptides are disclosed in Table 1, infra.

As used herein, the term “EphB receptor binding compound” refers to a molecule comprising an EphB receptor binding peptide that selectively or specifically binds to a member of the EphB family of receptors (e.g., EphB1, EphB2, EphB3, EphB4, EphB5 or EphB6). In a specific embodiment, an EphB receptor binding compound is a conjugate that selectively or specifically binds to a member of the EphB family of receptors (e.g., EphB1, EphB2, EphB3, EphB4, EphB5 or EphB6). In one embodiment, the conjugate comprises an EphB receptor binding peptide. In a specific embodiment, the conjugate comprises two or more EphB receptor binding peptide.

As used herein, the term “EphB receptor related disease” refers to any disease or pathological process involving one or more EphB receptors. Typically, the one or more EphB receptors associated with said EphB receptor related disease is aberrantly expressed, e.g., is inappropriately expressed, overexpressed, or underexpressed. In specific embodiments, the aberrant expression/activity of EphB receptors is determined using assays well known in the art, e.g., Western blot, immunohistochemistry, flow cytometry, ELISA, and transcriptional targer report assays. In such a disease context, the one or more EphB receptors associated with said EphB receptor related disease can also have aberrant receptor activity, e.g., overactivity or underactivity. Non-limiting examples of EphB receptor related diseases include neoplastic diseases, cancers, neurological diseases, and vascular diseases (e.g., macular degeneration). See Section 5.2, infra, for additional non-limiting examples of EphB receptor related diseases.

As used herein, the term “fragment” in the context of an amino acid sequence of an EphB receptor binding peptide, EphB receptor binding compound, or an Fc region of an immunoglobulin molecule refers to a molecule comprising an amino acid sequence of at least 4 contiguous amino acid residues, at least 5 contiguous amino acid residues, at least 8 contiguous amino acid residues, at least 10 contiguous amino acid residues, at least 20 contiguous amino acid residues, at least 30 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of an EphB receptor binding peptide, EphB receptor binding compound, or an Fc region of an immunoglobulin molecule, respectively.

As used herein, the term “fusion protein” refers to a conjugate having two proteinaceous molecules that are linked together via a peptide bond. In specific embodiments, a fusion protein refers to a polypeptide or protein that comprises two or more EphB receptor binding peptides; or a polypeptide or protein that comprises one or more EphB receptor binding peptides and a heterologous peptide linked together via a peptide bond. In a specific embodiment, a fusion protein does not comprise two or more EphB receptor binding peptides arranged as they are found contiguously in an EphB receptor or an EphrinB ligand amino acid sequence in the same N-terminal to C-terminal configuration. In a specific embodiment, a fusion protein comprises one or more EphB receptor binding peptides described herein (e.g., those identified by SEQ ID NOS:1-75 or disclosed in Table 1, infra) linked via a peptide bond to the Fc portion of the human IgG₁ immunoglobulin or a fragment thereof. Non-limiting examples of fusion proteins and methods for making the same are further discussed in Section 5.1, infra.

As used herein, the term “in combination” refers to the use of more than one therapies. The use of the term “in combination” does not restrict the order in which the therapies are administered to a subject. A first therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject. Any additional therapy can be administered in any order with the other additional therapies. In certain embodiments, an EphB receptor binding compound can be administered in combination with one or more therapies (e.g., non-EphB receptor binding compounds currently administered to prevent, treat, or manage a disease such as analgesic agents, anesthetic agents, antibiotics, cancer therapeutics, and immunomodulatory agents).

As used herein, the term “isolated” in the context of an organic or inorganic molecule (whether it be a small or large molecule), other than a proteinaceous agent or a nucleic acid, refers to an organic or inorganic molecule substantially free of a different organic or inorganic molecule. Preferably, an organic or inorganic molecule is 60%, 65%, 70%, 75%, 80%, 85%, 90%, 95%, or 99% free of a second, different organic or inorganic molecule. In a specific embodiment, an organic and/or inorganic molecule is isolated.

As used herein, the term “isolated” in the context of a proteinaceous agent (e.g., a peptide, polypeptide, fusion protein, or antibody) refers to a proteinaceous agent which is substantially free of cellular material or contaminating proteins from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous protein, polypeptide, or peptide (also referred to as a “contaminating protein”). When the proteinaceous agent is recombinantly produced, it is also preferably substantially free of culture medium, i.e., culture medium represents less than about 20%, 10%, or 5% of the volume of the proteinaceous agent preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. In a specific embodiment, proteinaceous agents disclosed herein are isolated. In another specific embodiment, an EphB receptor binding peptide or an EphB receptor binding compound is isolated.

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, is preferably substantially free of other cellular material, or culture medium when produced by recombinant techniques, or substantially free of chemical precursors or other chemicals when chemically synthesized. In certain embodiments, an “isolated” nucleic acid molecule is a nucleic acid molecule that is recombinantly expressed in a heterologous cell. In a specific embodiment, nucleic acid molecules are isolated. In another specific embodiment, a nucleic acid molecule encoding an EphB receptor binding peptide or an EphB receptor binding compound is isolated.

As used herein, the term “low tolerance” refers to a state in which the patient suffers from side effects from a therapy so that the patient does not benefit from and/or will not continue therapy because of the adverse effects and/or the harm from side effects outweighs the benefit of the therapy.

As used herein, the terms “manage,” “managing,” and “management” in the context of the administration of a therapy to a subject refer to the beneficial effects that a subject derives from a therapy, which does not result in a cure of the disease. In certain embodiments, a subject is administered one or more therapies to “manage” a disease so as to prevent the progression or worsening of the disease (i.e., hold disease progress).

As used herein, a “mimetic” or “peptidomimetic” of a peptide refers to a peptide that selectively or specifically binds to a member of the EphB family of receptors (e.g., EphB1, EphB2, EphB3, EphB4, EphB5 or EphB6) in which chemical structures of a peptide necessary for functional activity of the peptide have been replaced with other chemical structures which mimic the conformation of the peptide. Examples of peptidomimetics include peptidic compounds in which the peptide backbone is substituted with one or more benzodiazepine molecules (see, e.g., James et al., 1993, Science 260:1937-1942), peptides in which all L-amino acids are substituted with the corresponding D-amino acids and “retro-inverso” peptides (see, e.g., U.S. Pat. No. 4,522,752 by Sisto), described further below.

As used herein, the term “neoplastic” refers to a disease involving cells that have the potential to grow in an unregulated fashion and metastasize to distal sites and exhibit phenotypic traits that differ from those of non-neoplastic cells, for example, formation of colonies in a three-dimensional substrate such as soft agar or the formation of tubular networks or web-like matrices in a three-dimensional basement membrane or extracellular matrix preparation, such as MATRIGEL™. Non-neoplastic cells do not form colonies in soft agar and form distinct sphere-like structures in three-dimensional basement membrane or extracellular matrix preparations. Neoplastic cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. Thus, “non-neoplastic” means that the condition, disease, or disorder does not involve cells that do not have potential to metastasize.

As used herein, the phrase “non-responsive/refractory” is used to describe patients that are or have received one or more currently available therapies (e.g., cancer therapeutics) such as chemotherapy, radiation therapy, surgery, hormonal therapy and/or biological therapy/immunotherapy, particularly a standard therapeutic regimen for the particular cancer), wherein the therapy(ies) is not clinically adequate to treat the patients such that these patients need additional effective therapy, e.g., remain unsusceptible to therapy. The phrase can also describe patients who respond to therapy yet suffer from side effects, relapse, develop resistance, etc. In various embodiments, “non-responsive/refractory” means that at least some significant portion of the cancer cells are not killed or their cell division arrested. The determination of whether particular cells are “non-responsive/refractory” can be made either in vivo or in vitro by any method known in the art for assaying the effectiveness of treatment such cells, using the art-accepted meanings of “refractory” in such a context. In various embodiments, a cell is “non-responsive/refractory” where the number of cells has not been significantly reduced, or has increased during the therapy.

As used herein, the phrase “pharmaceutically acceptable” means approved by a regulatory agency of the federal or a state government, or listed in the U.S. Pharmacopeia, European Pharmacopeia, or other generally recognized pharmacopoeia for use in animals, and more particularly, in humans.

As used herein, “pharmaceutically acceptable salts” refer to the non-toxic alkali metal, alkaline earth metal, and ammonium salts commonly used in the pharmaceutical industry including the sodium, potassium, lithium, calcium, magnesium, barium, ammonium, and protamine zinc salts, which are prepared by methods well known in the art. The term also includes non-toxic acid addition salts, which are generally prepared by reacting the compounds of this invention with a suitable organic or inorganic acid. Representative salts include the hydrochloride, hydrobromide, sulfate, bisulfate, acetate, oxalate, valerate, oleate, laurate, borate, benzoate, lactate, phosphate, tosylate, citrate, maleate, fumarate, succinate, tartrate, napsylate, and the like.

As used herein, “pharmaceutically or therapeutically acceptable carrier” refers to a carrier medium which does not interfere with the effectiveness of the biological activity of the active ingredients and which is not toxic to the subject.

As used herein, the term “potentiate” refers to an improvement in the efficacy of a therapy at its common or approved dose.

As used herein, the terms “prevent,” “preventing,” and “prevention” in the context of the administration of a therapy to a subject refer to the preveption or inhibition of the recurrence, onset and/or development of a disease or one or more symptoms thereof resulting from the administration of a therapy (e.g., a prophylactic or therapeutic agent), or the administration of a combination of therapies (e.g., a combination of prophylactic or therapeutic agents). In some embodiments, such terms refer to one or more of the following: (1) an increase in the length of remission; (2) a decrease in the recurrence rate of the disease; and (3) an increase in the time to recurrence of the disease.

As used herein, the terms “prophylactic agent” and “prophylactic agents” refer to any agent(s) that can be used in the prevention of a disease. In certain embodiments, the term “prophylactic agent” refers to an EphB receptor binding compound. In certain other embodiments, the terms “prophylactic agent” and “prophylactic agents” refer to chemotherapeutics, radiation therapy, hormonal therapy, and/or biological therapy (e.g., immunotherapy). In other embodiments, more than one prophylactic agent may be administered in combination with other agents prophylactic and/or therapeutic agents.

As used herein, a “prophylactically effective amount” refers to that amount of a therapy sufficient to result in the prevention or inhibition of the recurrence, onset and/or development of an EphB receptor related disease. A prophylactically effective amount may refer to the amount of prophylactic agent sufficient to prevent the recurrence, onset and/or development of an EphB receptor related disease in a patient or subject, including but not limited to those patients predisposed to a such a disorder, for example those genetically predisposed or those having previously suffered from such a disorder. In one embodiment, a prophylactically effective amount is the amount of the prophylactic agent that provides a prophylactic benefit in the prevention of an EphB receptor related disease. In another embodiment, a prophylactically effective amount with respect to a prophylactic agent is the amount of prophylactic agent alone, or in combination with one or more other agents (e.g., non-EphB receptor binding compounds currently administered to treat the disease or disorder, analgesic agents, anesthetic agents, antibiotics, immunomodulatory agents) that provides a prophylactic benefit in the prevention of an EphB receptor related disease. Used in connection with an amount of an EphB receptor binding compound, the term can encompass an amount that improves overall prophylaxis or enhances the prophylactic efficacy of or synergies with another prophylactic agent.

As used herein, a “protocol” refers to a regimen for dosing, timing frequency and duration of administration of an agent or method for the prevention, treatment or management of a disease.

As used herein, the term “refractory” refers to a disease that is not responsive to a particular therapy. In a certain embodiment, that a disease is refractory to a therapy means that at least some significant portion of the symptoms associated with said disease is not eliminated or lessened by that therapy. The determination of whether a disease is refractory can be made either in vivo or in vitro by any method known in the art for assaying the effectiveness of a therapy for preventing, treating or managing a disease.

As used herein, the phrase “side effects” encompasses unwanted and adverse effects of a therapy. Adverse effects are always unwanted, but unwanted effects are not necessarily adverse. An adverse effect from a therapy might be harmful or uncomfortable or risky. Side effects from chemotherapy include, but are not limited to, gastrointestinal toxicity such as, but not limited to, early and late-forming diarrhea and flatulence, nausea, vomiting, anorexia, leukopenia, anemia, neutropenia, asthenia, abdominal cramping, fever, pain, loss of body weight, dehydration, alopecia, dyspnea, insomnia, dizziness, mucositis, xerostomia, and kidney failure, as well as constipation, nerve and muscle effects, temporary or permanent damage to kidneys and bladder, flu-like symptoms, fluid retention, and temporary or permanent infertility. Side effects from radiation therapy include but are not limited to fatigue, dry mouth, and loss of appetite. Side effects from biological therapies/immunotherapies include but are not limited to rashes or swellings at the site of administration, flu-like symptoms such as fever, chills and fatigue, digestive tract problems and allergic reactions. Side effects from hormonal therapies include but are not limited to nausea, fertility problems, depression, loss of appetite, eye problems, headache, and weight fluctuation. Additional undesired effects typically experienced by patients are numerous and known in the art. Many are described in the Physicians' Desk Reference (61^{st} ed., 2007).

As used herein, the term “selectively binds” and analogous terms in the context of EphB receptor binding peptides or EphB receptor binding compounds refer to a peptide, derivative, analog or conjugate that has a binding affinity for one or a few EphB receptor family members that is substantially greater than said binding affinity for the other EphB receptor family members and other antigens. As used in connection with selective binding affinity, “substantially greater” means at least a two-fold, at least a three-fold, at least a four-fold, at least a five-fold, at least a six-fold, at least a seven-fold, at least a eight-fold, at least a nine-fold, at least a ten-fold, at least a fifteen-fold, at least a twenty-fold, at least a thirty-fold, at least a forty-fold, at least a fifty-fold or at least a hundred-fold increase in the amount of peptide, derivative, analog or multimer bound to one or a few EphB receptor family members than to other Eph receptor family members and other antigens as measured by an immunoassay (e.g., an ELISA) or plasmon surface resonance.

As used herein, the term “specifically binds” and analogous terms in the context of EphB receptor binding peptides or EphB receptor binding compounds refers to a peptide, derivative, analog or conjugate that specifically binds to one member of the EphB family of receptors (e.g., EphB1, EphB2, EphB3, EphB4, EphB5 or EphB6) and not to other EphB receptors or Eph receptors of the A class, as measured by an immunoassay (e.g., an ELISA) or plasmon surface resonance.

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human. In one embodiment, the subject is a mammal, preferably a human, with an EphB receptor related disease. In another embodiment, the subject is a farm animal (e.g., a horse, pig, or cow), a pet (e.g., a guinea pig, dog or cat), or a laboratory animal (e.g., an animal model) with an EphB receptor related disease. In another embodiment, the subject is a mammal, preferably a human, at risk of developing an EphB receptor related disease. In a specific embodiment, the subject or patient is diagnosed with an EphB receptor related disease.

As used herein, the term “synergistic” refers to a combination of therapies (e.g., prophylactic or therapeutic agents) which is more effective than the additive effects of any two or more single therapies (e.g., one or more prophylactic or therapeutic agents). A synergistic effect of a combination of therapies (e.g., a combination of prophylactic or therapeutic agents) permits the use of lower dosages of one or more of therapies (e.g., one or more prophylactic or therapeutic agents) and/or less frequent administration of said therapies to a subject with an EphB receptor related disease. The ability to utilize lower dosages of therapies (e.g., prophylactic or therapeutic agents) and/or to administer said therapies less frequently reduces the toxicity associated with the administration of said therapies to a subject without reducing the efficacy of said therapies in the prevention or treatment of an EphB receptor related disease. In addition, a synergistic effect can result in improved efficacy of therapies (e.g., prophylactic or therapeutic agents) in the prevention or treatment of an EphB receptor related disease. Finally, synergistic effect of a combination of therapies (e.g., prophylactic or therapeutic agents) may avoid or reduce adverse or unwanted side effects associated with the use of any single therapy.

As used herein, the term “therapeutic agent” refers to any agent that can be used in the treatment and/or management of a disease or symptom thereof. In certain embodiments, the term “therapeutic agent” refers to an EphB receptor binding peptide or an EphB receptor binding compound. In certain other embodiments, the term “therapeutic agent” refers an agent other than an EphB receptor binding peptide or an EphB receptor binding compound. In particular embodiments, a therapeutic agent is an agent which is known to be useful for, or has been or is currently being used for the treatment and/or management of a disease, or one or more symptoms thereof.

As used herein, a “therapeutically effective amount” refers to that amount of a therapy sufficient to reduce or inhibit the progression, spread and/or duration of a disease in a subject, the reduction or amelioration of the severity of a disease, amelioration of one or more symptoms of a disease and/or the reduction in the duration of one or more symptom of a disease resulting from the administration of one or more therapies. In one embodiment, the therapeutically effective amount of a therapy is sufficient to eliminate, modify, or control a disease or a symptom associated with such a disease In another embodiment, the therapeutically effective amount is the amount of a therapy sufficient to delay or minimize the onset or severity of a disorder. In a specific embodiment, a therapeutically effective amount is the amount of the therapy that provides a therapeutic benefit in the treatment and/or management of a disorder. In another specific embodiment, a therapeutically effective amount with respect to a therapy means that amount of the therapy alone, or in combination with other therapies, that provides a therapeutic benefit in the treatment and/or management of a disorder. Used in connection with amount of an EphB receptor binding peptide or an EphB receptor binding compound, the term can encompass an amount that improves overall therapy, reduces or avoids unwanted effects, or enhances the therapeutic efficacy of or synergies with another therapy.

As used herein, the term “therapy” refers to any protocol, method and/or agent that can be used in the prevention, treatment and/or management of a disorder. In certain embodiments, the terms “therapies” and “therapy” refer to a biological therapy, supportive therapy, and/or other therapies useful in treatment, management and/or prevention or a disorder or one or more symptoms thereof known to one of skill in the art such as medical personnel.

As used herein, the terms “treat”, “treating” and “treatment” in the context of the administration of a therapy to a subject refer to the reduction or inhibition of the progression, spread and/or duration of a disease, the reduction or amelioration of the severity of a disease, amelioration of one or more symptoms of a disease, and/or the reduction in the duration of one or more symptom of a disease resulting from the administration of one or more therapies. In specific embodiments, such terms in the context of cancer refer to one, two, or three or more results following the administration of one, two, three or more therapies: (1) a reduction in the growth of a tumor or neoplasm; (2) a reduction in the formation of a tumor; (3) an eradication, removal, or control of primary, regional and/or metastatic cancer; (4) a reduction in metastatic spread; (5) a reduction in mortality; (6) an increase in survival rate; (7) an increase in length of survival; (8) an increase in the number of patients in remission; (9) a decrease in hospitalization rate; (10) a decrease in hospitalization lengths; and (11) the maintenance in the size of the tumor so that it does not increase by more than 10%, or by more than 8%, or by more than 6%, or by more than 4%; preferably the size of the tumor does not increase by more than 2%.

Concentrations, amounts, cell counts, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### 4. DESCRIPTION OF THE FIGURES

FIG. 1 is a graph showing that the TNYL-RAW peptide (SEQ ID NO:39) can be reacted with an activated PEG to form a PEG-linked multimeric peptide. The graph shows the average number of peptides per PEG linker molecule in four different experiments which varied by PEG size (3.4 kDa or 10 kDa) and initial peptide:PEG starting material ratio (3:1 or 5:1). The graph indicates that there are approximately 2 peptides per PEG linker in each sample, which supports the conclusion that PEG-linked peptide dimers are formed.

FIG. 2 is a graph showing that the PEG-linked TNYL-RAW-biotin dimers captured on wells coated with anti-PEG antibodies bind EphB4 Alkaline Phosphatase (“AP”). The data was obtained from experiments involving optical density (OD405) analysis of peptide dimers formed by conjugating TNYL-RAW-biotin peptide with PEG of either 3.4 kDa or 10 kDa. The coupling reaction was repeated by varying the initial PEG:peptide starting material ratio, which is shown in parentheses after each PEG peptide conjugate on the graph.

FIG. 3A-3D show stability data of the TNYL-RAW peptide in cell culture medium with PC3 cells (FIG. 3A and FIG. 3B), in cell conditioned medium in the absence of PC3 cells (FIG. 3C), and in cell conditioned medium without PC3 cells and with a mixture of protease inhibitors (FIG. 3D). In FIG. 5B, the cell culture medium was replaced with fresh medium just before addition of the TNYL-RAW peptide. In FIG. 3A, the cell culture medium was not replaced with fresh medium prior to addition of the TNYL-RAW peptide.

FIG. 4 is a graph showing that the TNYL-RAW-biotin peptide was successfully covalently coupled to bifunctional or monofunctional PEG. The PEGylated peptide was captured on wells coated with anti-PEG antibodies and detected with streptavidin-HRP. The data was obtained from experiments involving optical density analysis at OD405. The signal from peptide bound to bifunctional PEG was stronger than the background signal with peptide alone (dashed line) and the signal from peptide coupled to monofunctional PEG.

FIGS. 5A-5E present data from experiments to assess the functional characteristics of the TNYL-RAW Fc fusion protein. The TNYL-RAW Fc fusion protein comprises a signal peptide for secretion at the N-terminus followed by the TNYL-RAW peptide (underlined), followed by a GSGSK linker (SEQ ID NO:76) and the Fc domain (FIG. 5A). The TNYL-RAW Fc fusion protein purified from HEK293 cells were assayed for its ability to bind to EphB4 using ELISA plates. The TNYL-RAW Fc fusion protein was immobilized on protein A ELISA plates and detected with EphB4 conjugated to Alkaline Phosphatase (AP) (FIG. 5B). The binding affinity of the TNYL-RAW Fc fusion protein for EphB4-AP (FIG. 5C) was compared with the binding affinity of the ephrin B2 Fc fusion protein (FIG. 5D). The ability of the TNYL-RAW Fc fusion protein to stimulate MDA-MB-231 cells was assayed by incubating 2 μg/ml TNYL-RAW Fc protein for 20 minutes or 1 μg/ml ephrin B2 Fc protein (positive control) or human Fc domain (negative control), subjecting the cell lysates to EphB4 immunoprecipitation, and immunoblotting with anti-phosphotyrosine antibodies (PTyr) (FIG. 5E). Reprobing by immunoblotting with anti-EphB4 antibodies was done as a control.

### 5. DETAILED DESCRIPTION OF THE INVENTION

The Eph receptor tyrosine kinases are overexpressed in many pathologic tissues and have therefore emerged as promising drug target candidates. However, there are few molecules available that can selectively bind to a single Eph receptor and not other members of this large receptor family. In one embodiment, EphB receptor binding peptides and EphB receptor binding compounds selectively bind to different receptors of the EphB class, including but not limited to, EphB1, EphB2, EphB3, EphB4, EphB5, and EphB6. In specific embodiments, the EphB receptor binding peptides and EphB receptor binding compounds contain amino acid motifs found in the G-H loop of the Ephrin-B ligands, which is the region that mediates high-affinity interaction with the EphB receptors. Consistent with targeting the Ephrin-binding site, the higher-affinity multimeric peptides may act as agonists or antagonists of the EphB receptors to which they bind.

EphB receptors, and in particular, EphB2 and EphB4 (see Section 2, supra), are overexpressed in a wide variety of cancers, and compounds that bind to these receptors would thus be useful for imaging and drug targeting applications. In specific embodiments, EphB receptor binding compounds may act as EphB receptor agonists and are thus useful for cancer therapy, to reduce cell proliferation and/or metastasis. The interplay between EphB4 expressed in breast cancer cells and Ephrin-B2 expressed in the tumor vasculature was reported wherein the cytoplasmic domain of the transmembrane Ephrin-B2 ligand promoted tumor growth by stimulating angiogenesis (Noren et al., 2004, PNAS USA 101:5583-5588). Thus, in another specific embodiment, the EphB receptor binding compounds may act as EphB receptor antagonists, to inhibit activation of EphB receptors expressed in the vascular endothelial tissues, thus inhibiting angiogenesis and inhibiting tumor growth.

In some embodiments, EphB receptor binding peptides and EphB receptor binding compounds are used as therapies Eph receptor related diseases. Non-limiting examples of such diseases include neoplastic diseases, cancers, neurological diseases, and vascular diseases (e.g., macular degeneration). In some embodiments, the Eph receptor related disease is an EphB receptor related diseases. An EphB receptor related disease includes those diseases associated with higher than normal expression of the EphB receptor. In a specific embodiment, a higher than normal expression of the EphB receptor is at least 10%, at least 20%, at least 30%, at least 40%, at least 50%, at least 60%, at least 70%, at least 80% at least 90%, or at least 100% higher than normal expression of the EphB receptor of a control (e.g., serum, cells, and tissue samples from a normal healthy subject) as determine via assays well known in the art, e.g., immunofluorescence, ELISA, flow cytometry, and Western. In another specific embodiment, a higher than normal expression of the EphB receptor is at least 1.5 fold, at least 2 fold, at least 3 fold, at least 4 fold, at least 5 fold, at least 6 fold, at least 7 fold, at least 8 fold at least 9 fold, or at least 10 fold higher than normal expression of the EphB receptor of a control (e.g., serum, cells, and tissue samples from a normal healthy subject) as determine via assays well known in the art, e.g., immunofluorescence, ELISA, flow cytometry, and Western. Accordingly, in a specific embodiment, EphB receptor binding peptides and EphB receptor binding compounds targeting the EphB receptor could have therapeutic effectiveness against such EphB receptor related diseases. Examples of EphB receptor related diseases include neoplastic diseases, cancer, neurological diseases (e.g., spinal cord injury), and vascular diseases (e.g., macular degeneration).

The present invention provides peptides (i.e., EphB receptor binding peptides) and compounds (i.e., EphB receptor binding compounds), which selectively bind to a member of the EphB receptor family, including, but not limited to, EphB1, EphB2, EphB3, EphB4, EphB5 and EphB6. In one embodiment, an isolated EphB receptor binding peptide has the amino acid sequence of SEQ ID NO:41. In specific embodiments, the EphB receptor binding peptides and EphB receptor binding compounds compete with and/or inhibit binding of an Ephrin-B ligand (e.g., Ephrin-B1, Ephrin-B2 and Ephrin-B3) to an EphB receptor.

Presented herein are isolated multimeric peptides comprising two or more EphB receptor binding peptides, which multimeric peptides selectively bind to a member of the EphB receptor family with a dissociation constant (Kd) of about 100 nM or less, about 71 nM or less, or about 20 nM or less, and inhibit the binding of the EphB receptor to an EphrinB ligand. In another embodiment, the multimeric peptides selectively bind to a member of the EphB receptor family with a dissociation constant (Kd) of about 15 nM or less; 10 nM or less; or 5 nM or less. In one embodiment, the isolated multimeric peptides inhibit the binding of the EphB receptor to an EphrinB ligand at an IC₅₀ of about 100 nM or less. In some embodiment, the isolated multimeric peptides inhibit the binding of the EphB receptor to an EphrinB ligand at an IC₅₀ of about 20 nM or less or 15 nM or less. In a specific embodiment, the isolated multimeric peptides are dimers. In some embodiments, the dimers are produced by linking the two EphB receptor binding peptides using polyethylene glycol (PEG). In another embodiment, the isolated multimeric peptides comprise two or more EphB receptor binding peptides that are the same. In a particular embodiment, the isolated multimeric peptides comprise two or more EphB receptor binding peptides wherein at least one of the EphB receptor binding peptides differ from the other EphB receptor binding peptide(s). In a certain embodiment, the EphB receptor is EphB1, EphB2, EphB3, EphB4, EphB5, or EphB6. In other embodiments, the EphB receptor binding peptides has the amino acid sequence of any of SEQ ID NOS:1-75. In one embodiment, the isolated multimeric peptides are agonistic. In another embodiment, the isolated multimeric peptides are antagonistic. In specific embodiments, the EphB receptor binding compound which selectively binds to an EphB receptor of the EphB receptor family with a dissociation constant (Kd) of about 100 nM or less is an isolated conjugate comprising (i) two or more peptides that selectively binds to the EphB receptor and each of said two or more peptides has a length of between 5 to 50 amino acid residues, and (ii) a heterologous compound.

In a certain embodiment, an isolated multimeric peptide which selectively binds to a member of the EphB receptor family comprises two or more EphB receptor binding peptides, and the multimeric peptide inhibits the binding of the EphB receptor to an EphrinB ligand at an IC₅₀ of about 100 nM or less. In a specific embodiment, the isolated multimeric peptide is a dimer. In another specific embodiment, the dimer is produced by linking the two EphB receptor binding peptides using polyethylene glycol (PEG). In another embodiment, the isolated multimeric peptide comprise two or more EphB receptor binding peptides that are the same. In a particular embodiment, the isolated multimeric peptides comprise two or more EphB receptor binding peptides wherein at least one of the EphB receptor binding peptides differ from the other EphB receptor binding peptide(s). In certain embodiment, the EphB receptor is EphB1, EphB2, EphB3, EphB4, EphB5, or EphB6. In other embodiments, the EphB receptor binding peptides is SEQ ID NOS:1-75.

Presented herein are isolated conjugates comprising an EphB receptor binding peptide and a heterologous compound, wherein isolated conjugates inhibit the binding of the EphB receptor to an EphrinB ligand, and the conjugates selectively binds to an EphB receptor with a dissociation constant (Kd) of about 100 nM or less, about 71 nM or less, or about 20 nM or less. In some embodiments, the isolated conjugates have a dissociation constant (Kd) of about 15 nM or less, 10 nM or less, or 5 nM or less. In certain embodiments, an isolated conjugate, comprising an EphB receptor binding peptide and a heterologous compound, inhibits the binding of the EphB receptor to an EphrinB ligand at an IC₅₀ of about 100 nM or less. In other embodiments, the isolated conjugates inhibit the binding of the EphB receptor to an EphrinB ligand at an IC₅₀ of about 50 nM or less, 40 nM or less, 30 nM or less, 20 nM or less, or 15 nM or less. In a specific embodiment, the heterologous compound is the Fc region of an IgG or a fragment thereof (e.g., CH2 or CH3 domain). In a certain embodiment, the heterologous compound is polyethylene glycol (PEG). In some embodiments, the isolated conjugate is a fusion protein. In certain embodiment, the EphB receptor is EphB1, EphB2, EphB3, EphB4, EphB5, or EphB6. In other embodiments, the EphB receptor binding peptides is SEQ ID NOS:1-75.

Presented herein are isolated multimeric peptides comprising at least two EphB receptor binding peptides having the amino acid sequence of SEQ ID NO:39. In some embodiments, the isolated multimeric peptides comprise at least two EphB receptor binding peptides having the amino acid sequence of SEQ ID NO:40. In other embodiments, the isolated multimeric peptides comprise at least two EphB receptor binding peptides having the amino acid sequence of SEQ ID NO:41. In a specific embodiment, the isolated multimeric peptides are dimers. In another embodiment, the multimeric peptides have a dissociation constant (Kd) of about 15 nM or less, 10 nM or less, or 5 nM or less. In another embodiment, isolated multimeric peptides inhibit the binding of the EphB receptor to an EphrinB ligand at an IC₅₀ of about 75 nM or less, about 50 nM or less, about 25 nM or less, about 15 nM or less, about 10 nM or less, or about 5 nM or less.

The present invention also provides compositions, including pharmaceutical compositions, comprising the EphB receptor binding peptides and/or the EphB receptor binding compounds and a pharmaceutically acceptable carrier or excipient. The peptides and compounds are useful for the prevention, treatment and/or management of EphB receptor related diseases. The peptides and compounds are also useful in the diagnosis and/or monitoring of EphB receptor related diseases as well as in methods for identifying compounds that selectively or specifically bind to a member of the EphB receptor family.

In one embodiment, compositions comprise a pharmaceutically acceptable carrier or excipient and a multimeric peptide, wherein the multimeric peptide comprises two or more EphB receptor binding peptides and selectively binds to a member of the EphB receptor family with a dissociation constant (Kd) of approximately 100 nM or less, approximately 71 nM or less, approximately 40 nM or less, or approximately 20 nM or less and inhibits the binding of the EphB receptor. In a specific embodiment, the composition further comprises a chemotherapy, a hormonal therapy, a radiation therapy, a biological therapy or a immunotherapy.

In another embodiment, a composition comprises a pharmaceutically acceptable carrier or excipient and an isolated conjugate that selectively binds to an EphB receptor with a dissociation constant (Kd) of approximately 100 nM or less, approximately 71 nM or less, approximately 40 nM or less, or approximately 20 nM or less and inhibits the binding of EphB receptor to an Ephrin B ligand, wherein the conjugate comprises an EphB receptor binding peptide and a heterologous compound. In some embodiments, a composition comprises a pharmaceutically acceptable carrier or excipient and an isolated conjugate that selectively binds to an EphB receptor with a dissociation constant (Kd) of approximately 15 nM or less, 10 nM or less, 5 nM or less, or 1 nM or less and inhibits the binding of EphB receptor to an Ephrin B ligand, wherein the conjugate comprises an EphB receptor binding peptide and a heterologous compound. In a specific embodiment, the composition further comprises a chemotherapy, a hormonal therapy, a radiation therapy, a biological therapy or a immunotherapy.

In other embodiments, the invention provides for compositions, including pharmaceutical compositions, comprising an EphB receptor binding peptide having SEQ ID NO:41 and a pharmaceutically acceptable carrier or excipient. In a specific embodiment, the composition further comprises a chemotherapy, a hormonal therapy, a radiation therapy, a biological therapy or a immunotherapy.

The invention provides for methods of preventing, treating or managing an EphB receptor-related disease, the method comprising administering to a subject in need thereof a prophylactically or therapeutically effective amount of a multimeric peptide, that selectively binds to a member of the EphB receptor family with a dissociation constant (Kd) of approximately 100 nM or less, approximately 71 nM or less, approximately 40 nM or less, or approximately 20 nM or less and inhibits the binding of the EphB receptor to an EphrinB ligand, wherein the multimeric peptide comprises two or more EphB receptor binding peptides. In some embodiments, the method comprising administering to a subject in need thereof a prophylactically or therapeutically effective amount of a multimeric peptide, that selectively binds to a member of the EphB receptor family with a dissociation constant (Kd) of approximately 15 nM or less, 10 nM or less, or 5 nM or less and inhibits the binding of the EphB receptor to an EphrinB ligand, wherein the multimeric peptide comprises two or more EphB receptor binding peptides. In a particular embodiment, the EphB receptor-related disease is a neoplastic disease, a vascular disease, or a neurological disorder. In other embodiments, the EphB receptor-related disease is cancer, e.g., mesothelioma, ovarian cancer, bladder cancer, squamous cell carcinoma of the head and neck, breast cancer, prostate cancer, colon cancer, small cell lung carcinoma, and a cancer of neurological origin. In specific embodiments, the method of preventing, treating or managing cancer further comprises administering to the subject a chemotherapy, a hormonal therapy, a radiation therapy, a biological therapy or an immunotherapy. In one embodiment, the subject is a human.

In certain embodiment, a method of preventing, treating or managing an EphB receptor-related disease comprises administering to a subject in need thereof a prophylactically or therapeutically effective amount of an isolated conjugate that selectively binds to an EphB receptor with a dissociation constant of 20 nM or less and inhibits the binding of the EphB receptor to an Ephrin B ligand wherein the conjugate comprises an EphB receptor binding peptide and a heterologous compound. In some embodiments, the method comprises administering to a subject in need thereof a prophylactically or therapeutically effective amount of an isolated conjugate that selectively binds to an EphB receptor with a dissociation constant of 15 nM or less, 10 nM or less, or 5 nM or less and inhibits the binding of the EphB receptor to an Ephrin B ligand wherein the conjugate comprises an EphB receptor binding peptide and a heterologous compound. In a particular embodiment, the EphB receptor-related disease is a cancer, aneoplastic disease, a vascular disease, or a neurological disorder. In other embodiments, the EphB receptor-related disease is cancer, e.g., mesothelioma, ovarian cancer, bladder cancer, squamous cell carcinoma of the head and neck, breast cancer, prostate cancer, colon cancer, small cell lung carcinoma, and a cancer of neurological origin. In specific embodiments, the method of preventing, treating or managing cancer further comprises administering to the subject a chemotherapy, a hormonal therapy, a radiation therapy, a biological therapy or an immunotherapy. In one embodiment, the subject is a human.

In certain embodiments, a method of preventing, treating or managing an EphB receptor-related disease comprises administering to a subject in need thereof a prophylactically or therapeutically effective amount of the EphB receptor binding peptide which is agonistic. In a particular embodiment, the EphB receptor-related disease is a neoplastic disease, a vascular disease, or a neurological disorder. In other embodiments, the EphB receptor-related disease is cancer, e.g., mesothelioma, ovarian cancer, bladder cancer, squamous cell carcinoma of the head and neck, breast cancer, prostate cancer, colon cancer, small cell lung carcinoma, and a cancer of neurological origin. In specific embodiments, the method of preventing, treating or managing cancer further comprises administering to the subject a chemotherapy, a hormonal therapy, a radiation therapy, a biological therapy or an immunotherapy. In one embodiment, the subject is a human.

The invention provides methods of treating cancer comprising administering to a subject in need thereof a therapeutically effective amount of an isolated EphB receptor binding compound following removal or a tumor from the subject. In other embodiments, the invention provides for methods of preventing or treating a relapse of cancer comprising administering to a subject in need thereof a prophylactically or therapeutically effective amount of an isolated EphB receptor binding compound. In particular embodiments, the invention provides for methods of reducing the number of EphB receptor expressing cells (e.g., cells that aberrantly express an EphB receptor) and methods of inhibiting proliferation of EphB receptor expressing cells (e.g., cells that aberrantly express an EphB receptor), said methods comprising contacting the cells with an effective amount of an isolated EphB receptor binding compound. The invention also provides for methods of reducing the size of a tumor and methods of preventing growth of a tumor, said methods comprising contacting the tumor with an effective amount of an isolated EphB receptor binding compound.

In other embodiments, the invention also provides for methods for detecting aberrant expression of an EphB receptor in a subject, the methods comprising (a) contacting samples or cells of the subject with an isolated EphB receptor binding compound comprising a detectable agent; and (b) detecting binding of the isolated EphB receptor binding compound to the samples or cells of the subject, wherein aberrant expression of the EphB receptor is detected if the binding of the isolated EphB receptor binding compound to said samples of the subject is higher or lower than the binding of the isolated EphB receptor binding compound to control samples or cells that have normal expression of the EphB receptor. In specific embodiments, the level of EphB receptor expression is measured in a subject and is compared to the level of EphB receptor expression in a healthy subject (e.g., a normal level of EphB receptor expression) or subjects who does not have a detected EphB receptor related disease, or to a predetermined reference range for a healthy subject or a subject who does not have a detectable EphB receptor related disease. In other embodiments, methods of detecting aberrant expression of an EphB receptor in a subject comprise measuring the level of EphB receptor expression in a sample of the subject or in the subject and comparing the level of EphB receptor expression in the sample or the subject to the level of EphB receptor expression in a healthy subject (e.g., a normal level of EphB receptor expression) or subjects who does not have a detected EphB receptor related disease, or to a predetermined reference range for a subject that aberrantly expresses an EphB receptor or has an EphB receptor related disease, wherein aberrant expression of an EphB receptor is detected if there is an equivalent or greater level of EphB receptor expression in the sample or the subject relative to the predetermined reference range. In certain embodiments, methods of detecting aberrant expression of an EphB receptor is for the purpose of diagnosing an EphB receptor related disease. In other embodiments, methods of detecting aberrant expression of an EphB receptor is for the purpose of monitoring the progression of an EphB receptor related disease or for the purpose of monitoring the effectiveness of a therapy.

5.1 EPHB Receptor Binding Peptides and EPHB Receptor Binding Compounds

The present invention provides peptides and compounds that selectively bind to a member of the EphB receptor family, including, but not limited to, EphB1, EphB2, EphB3, EphB4, EphB5, and EphB6. In some embodiments, EphB receptor binding peptides compete with and/or inhibit the binding of an EphB receptor to an EphrinB ligand. In certain embodiments, the EphB receptor binding peptides are antagonists. In a some embodiments, the EphB receptor binding peptides are agonists. In specific embodiments, the EphB receptor binding peptide is 4, 5, 6, 7, 8, 9, 10, 11, 12, 13, 14, 15, 16, or 17 amino acid residues in length. In other embodiments, the EphB receptor binding peptide is less than 10, less than 15, less than 20, less than 25, less than 30, less than 35, less than 40, less than 45 or less than 50 amino acid residues in length with a minimum of 4 or 5 amino acid residues. In other embodiments, the EphB receptor binding peptide has the motif ΦxxΦ (where “x” is a non-conserved amino acid and 1 is an aromatic amino acid (Aasland et al., 2002, FEBS Lett 513:141-144)), which is also found in the G-H loop of certain Ephrin ligands.

In certain embodiments, the EphB receptor binding peptides have a high binding affinity for a member of the EphB class of receptors, e.g., EphB1, EphB2, EphB3, EphB4, EphB5 or EphB6. In specific embodiments, the EphB receptor binding peptides have specific association rate constants (kₒₙ values), dissociation rate constants (k_{off} values), affinity constants (Kₐ values), dissociation constants (K_{d} values) and/or IC₅₀ values.

In a specific embodiment, an EphB receptor binding peptide selectively binds to an EphB receptor with a kₒₙ rate of at least 10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹.

In another embodiment, an EphB receptor binding peptide selectively binds to an EphB receptor with a k_{off} rate of 5×10⁻¹ s⁻¹ or less, 10⁻¹ s⁻¹ or less, 5×10⁻² s⁻¹ or less, 10⁻² s⁻¹ or less, 5×10⁻³ s⁻¹ or less, 10⁻³ s⁻¹ or less, 5×10⁴ s⁻¹ or less, 10⁴ s⁻¹ or less, 5×10⁻⁵ s⁻¹ or less, 10⁻⁵ s⁻¹ or less, 5×10⁻⁶ s⁻¹ or less, 10⁻⁶ s⁻¹ or less, 5×10⁻⁷ s⁻¹ or less, 10⁻⁷ s⁻¹ or less, 5×10⁻⁸ s⁻¹ or less, 10⁻⁸ s⁻¹ or less, 5×10⁻⁹ s⁻¹ or less, 10⁻⁹ s⁻¹ or less, 5×10⁴⁰ s⁻¹ or less, or 10⁻¹° s⁻¹ or less.

In another embodiment, an EphB receptor binding peptide selectively binds to an EphB receptor with a Kₐ (kₒₙ/k_{off}) of at least 10¹¹ nM⁻¹, at least 5×10¹¹ nM⁻¹, at least 10¹² nM⁻¹, at least 5×10¹² nM⁻¹, at least 10¹³ nM⁻¹, at least 5×10¹³ nM⁻¹, at least 10¹⁴ nM⁻¹, at least 5×10¹⁴ nM⁻¹, at least 10¹⁵ nM⁻¹, at least 5×10¹⁵ nM⁻¹, at least 10¹⁶ nM⁻¹ at least 5×10¹⁶ nM⁻¹, at least 10¹⁷ nM⁻¹, at least 5×10¹⁷ nM⁻¹, at least 10¹⁸ nM⁻¹, at least 5×10¹⁸ nM⁻¹, at least 10¹⁹ nM⁻¹, at least 5×10¹⁹ nM⁻¹, at least 10²⁰ nM⁻¹, at least 5×10²⁰ nM⁻¹, at least 10²¹ nM⁻¹, at least 5×10²¹ nM⁻¹, at least 10²² nM⁻¹, at least 5×10²² nM⁻¹, at least 10²³ nM⁻¹, at least 5×10²³ nM⁻¹, at least 10²⁴ nM⁻¹, or at least 5×10²⁴ nM⁻¹.

In other embodiments, an EphB receptor binding peptide selectively binds to an EphB receptor with a K_{d} (k_{off}/kₒₙ) of 5×10⁷ nM or less, 10⁷ nM or less, 5×10⁶ nM or less, 10⁶ nM or less, 5×10⁵ nM or less, 10⁵ nM or less, 5×10⁴ nM or less, 10⁴ nM or less, 5×10³ nM or less, 10³ nM or less, 5×10² nM or less, 100 nM or less, 90 nM or less, 80 nM or less, 70 nM or less, 60 nM or less, 50 nM or less, 20 nM or less, 15 nM or less, 10 nM or less, 5 nM or less, 3.8 nM or less, 2 nM or less, 1.5 nM or less, 1 nM or less, 5×10⁻¹ nM or less, 10⁻¹ nM or less, 5×10⁻² nM or less, 10⁻² nM or less, 5×10⁻³ nM or less, 10⁻³ nM or less, 5×10⁻⁴ nM or less, 10⁻⁴ nM or less, 5×10⁻⁵ nM or less, 10⁻⁵ nM or less, 5×10⁻⁶ nM or less, or 10⁻⁶ nM or less. In specific embodiments, an EphB receptor binding peptide selectively binds to an EphB receptor with a K_{d} (k_{off}/kₒₙ) between approximately 1 nM and approximately 10 nM, or approximately 1 nM and approximately 20 nM, or approximately 1 nM and approximately 30 nM, or approximately 1 nM and approximately 40 nM, or approximately 1 nM and approximately 50 nM, or approximately 1 nM and approximately 60 nM, or approximately 1 nM and approximately 70 nM, or approximately 1 nM and approximately 80 nM, or approximately 1 nM and approximately 90 nM, or approximately 1 nM and approximately 100 nM, or between approximately 0.5 nM and approximately 1 nM, or between approximately 0.5 nM and approximately 10 nM, or between approximately 0.5 nM and approximately 20 nM, or between approximately 0.5 nM and approximately 30 nM, or between approximately 0.5 nM and approximately 40 nM, or between approximately 0.5 nM and approximately 50 nM, or between approximately 0.5 nM and approximately 60 nM, or between approximately 0.5 nM and approximately 70 nM, or between approximately 0.5 nM and approximately 80 nM, or between approximately 0.5 nM and approximately 90 nM, or between approximately 50 nM and approximately 80 nM, or between approximately 60 nM and approximately 90 nM, or between approximately 50 nM and approximately 100 nM, or between approximately 0.6 nM and approximately 1.1 nM, or between approximately 0.7 nM and approximately 1.2 nM, or between approximately 0.5 and approximately 5 nM. In other specific embodiments, an EphB receptor binding peptide selectively binds to an EphB receptor with a K_{d} (k_{off}/kₒₙ) of about 71 nM, about 70 nM, about 5 nM, about 3.5 nM, about 1.2 nM, about 1.1 nM, about 1 nM, about 0.9 nM, or about 0.8 nM. In specific embodiments, the K_{d} (k_{off}/kₒₙ) value is determined by assays well known in the art or described herein, e.g., ELISA, isothermal titration calorimetry (ITC), BIAcore, or fluorescent polarization assay. In particular embodiments, an EphB receptor binding peptide selectively binds to a murine EphB receptor (or an ectodomain of an EphB receptor) with a K_{d} value that is determined by assays well known in the art or described herein, e.g., ELISA, isothermal titration calorimetry (ITC), BIAcore, or fluorescent polarization assay. In particular embodiments, an EphB receptor binding peptide selectively binds to a human EphB receptor (or an ectodomain of an EphB receptor) with a K_{d} value that is determined by assays well known in the art or described herein, e.g., ELISA, isothermal titration calorimetry (ITC), BIAcore, or fluorescent polarization assay. In specific embodiments, the K_{d} value of an EphB receptor binding peptide for a murine EphB receptor is measured using assays well known in the art or described herein, e.g., ELISA, isothermal titration calorimetry (ITC), BIAcore, or fluorescent polarization assay. In certain embodiments, the K_{d} value of an EphB receptor binding peptide for a human EphB receptor is measured using assays well known in the art or described herein, e.g., ELISA, isothermal titration calorimetry (ITC), BIAcore, or fluorescent polarization assay.

In some embodiments, an EphB receptor binding peptide selectively binds to an EphB receptor and inhibits the binding of an EphB receptor to an Ephrin B ligand at an IC₅₀ value of less than 5×10⁷ nM, less than 10⁷ nM, less than 5×10⁶ nM, less than 10⁶ nM, less than 5×10⁵ nM, less than 10⁵ nM, less than 5×10⁴ nM, less than 10⁴ nM, less than 5×10³ nM, less than 10³ nM, less than 5×10² nM, less than 100 nM, less than 90 nM, less than 80 nM, less than 70 nM, 69 nM, less than 60 nM, less than 50 nM, less than 40 nM, less than 30 nM, less than 25 nM, less than 20 nM, less than 15 nM, less than 12 nM, less than 10 nM, less than 5 nM, less than 1 nM, less than 5×10⁻¹ nM, less than 10⁻¹ nM, less than 5×10⁻² nM, less than 10⁻² nM, less than 5×10⁻³ nM, less than 10⁻³ nM, less than 5×10⁴ nM, or less than 10⁻⁴ nM when measured according to methods well known in the art or described herein, e.g., ELISA. In other embodiments, an EphB receptor binding peptide binds to EphB4 and has an approximate IC₅₀ value of between approximately 1 nM and approximately 10 nM, between approximately 1 nM and approximately 15 nM, between approximately 1 nM and approximately 20 nM, between approximately 1 nM and approximately 25 nM, between approximately 1 nM and approximately 30 nM, between approximately 1 nM and approximately 40 nM, between approximately 1 nM and approximately 50 nM, between approximately 10 nM and approximately 10² nM, between approximately 10² nM and approximately 10³ nM, between approximately 10³ nM and approximately 10⁴ nM, between approximately 10⁴ nM and approximately 10⁵ nM, between approximately 10⁵ nM and approximately 10⁶ nM, or between approximately 10⁶ nM and approximately 10⁷ nM when measured according to methods well known in the art or described herein, e.g., ELISA. In a specific embodiment, an EphB receptor binding peptide binds to EphB4 and has an approximate IC₅₀ value of between approximately 5 nM and approximately 10 nM, between approximately 5 nM and approximately 15 nM, between approximately 10 nM and approximately 15 nM, between approximately 10 nM and approximately 20 nM, between approximately 10 nM and approximately 30 nM, between approximately 10 nM and approximately 40 nM, between approximately 10 nM and approximately 50 nM, between approximately 1 nM and approximately 100 nM, between approximately 10 nM and approximately 100 nM, between approximately 20 nM and approximately 100 nM, between approximately 30 nM and approximately 100 nM, between approximately 40 nM and approximately 100 nM, between approximately 50 nM and approximately 100 nM, between approximately 15 nM and approximately 25 nM, or between approximately 15 nM and approximately 20 nM when measured according to methods well known in the art or described herein, e.g., ELISA. In specific embodiments, the IC₅₀ value is determined for an EphB receptor binding peptide that inhibits a murine Ephrin B ligand binding to a murine EphB receptor. In particular embodiments, the ICₛₒ is determined for an EphB receptor binding peptide that inhibits a murine Ephrin B ligand binding to a human EphB receptor. In some embodiments, the IC₅₀ value is determined for an EphB receptor binding peptide that inhibits the binding of a human Ephrin B ligand to a murine EphB receptor. In other embodiments, the IC₅₀ value is determined for an EphB receptor binding peptide that inhibits the binding of a human Ephrin B ligand to a human EphB receptor. In specific embodiments, the IC₅₀ value is determined for an EphB receptor binding peptide that inhibits the binding of a murine Ephrin B ligand to a murine EphB receptor using an ELISA. In specific embodiments, the IC₅₀ value is measured, e.g., by ELISA, using a preparation of EphB receptor binding peptides that has a purity of approximately 20%, approximately 30%, approximately 40%, approximately 50%, approximately 60%, approximately 70%, approximately 80%, approximately 90%, approximately 95%, approximately 99%. In particular embodiments, an EphB receptor binding peptide selectively binds to an EphB receptor and inhibits the binding of an EphB receptor to an Ephrin B ligand at an IC₅₀ value as measured in an assay, e.g., by ELISA, wherein the concentration of soluble Ephrin B ligand or EphB receptor used in the assay is approximately 0.001 μM, 0.005 μM, 0.01 μM, 0.05 μM, 0.1 μM, 0.5 μM, 1 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1000 μM, or 5000 μM.

Non-limiting examples of EphB receptor binding peptides are provided in Table 1. In a specific embodiment, the EphB receptor binding peptide is TNYLFSPNGPIARAW (“TNYL-RAW,” SEQ ID NO: 39). In another embodiment, the EphB receptor binding peptide is NYLFSPNGPIARAW (“NYL-RAW,” SEQ ID NO: 40). In another embodiment, the EphB receptor binding peptide is YLFSPNGPIARAW (“YL-RAW,” SEQ ID NO: 41).

The invention provides compounds which selectively bind to a member of the EphB receptor family (i.e., EphB receptor binding compounds). In a specific embodiment, EphB receptor binding compounds compete with and/or inhibit the binding of an EphB receptor to an ephrin B ligand. In some embodiments, the EphB receptor binding compounds are agonistic. In other embodiments, the EphB receptor binding compounds are antagonistic.

The EphB receptor binding peptides described above can be conjugated (e.g., fused or linked) as described herein to provide EphB receptor binding compounds which have improved properties, such as, e.g., improved potency, activity, selectivities, binding affinities and/or improved half-lives.

In specific embodiments, the EphB receptor binding compounds described herein have an activity greater than an EphB receptor binding peptide that is monomeric and is not conjugated or otherwise modified (e.g., a peptide described in Table 1). In some embodiments, the EphB receptor binding compounds described herein have an activity that is about 0.1 to about 0.01-fold that of the EphB receptor binding peptide. In other embodiments, the EphB receptor binding compounds described herein have an activity that is about 0.1 to 1-fold that of the EphB receptor binding peptide. For example, in certain embodiments, the EphB receptor binding compounds have improved association rate constants (kₒₙ values), dissociation rate constants (k_{off} values), affinity constants (Kₐ values) and/or dissociation constants (K_{d} values) than EphB receptor binding peptides (e.g., those in Table 1). In other embodiments, the EphB receptor binding compounds have improved IC₅₀ values compared to a monomeric peptide (e.g., as described in Table 1). In other embodiments, the EphB receptor binding compounds cause greater death (e.g., greater death rates) of cancer cells in vitro than EphB receptor binding peptides (e.g., those in Table 1) when analyzed using standard assays known in the art to measure cell survival and/or growth; for example, cell proliferation can be assayed by measuring ³H-thymidine incorporation, flow cytometry, by direct cell count, by detecting changes in transcriptional activity of known genes such as proto-oncogenes (e.g., fos, myc) or cell cycle markers; cell viability can be assessed by trypan blue staining. In accordance with these embodiments, the term “greater” in the context of cancer cell death (e.g., greater death rates), refers to the percentage of cell death caused by contacting the cancer cells with an EphB receptor binding peptide or an EphB receptor binding compound, as compared to contacting the cancer cells with a control peptide or compound using assays well known in the art and/or assays described herein. Thus, an EphB receptor binding peptide or an EphB receptor binding compound can cause at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% greater death (e.g., greater death rates) of cancer cells relative to a control peptide or compound as determined by assays well known in the art and/or assays described herein, e.g., ³H-thymidine incorporation assay, cell count assay, cell viability assay with trypan blue staining. In another embodiment, an EphB receptor binding peptide or an EphB receptor binding compound can cause at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% greater reduction in the size of a tumor relative to a control peptide or compound as determined by assays well known in the art and/or assays described herein, e.g., physical examination of the tumor, imaging analysis (e.g., MRI, CT-Scan, X-ray).

In specific embodiments, an EphB receptor binding compound has at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of the activity of an EphB receptor binding peptide. In a specific embodiment, an EphB receptor binding compound has an amount of activity that is at least 1 fold, at least 1.5 fold, at least 2 fold, at least 2.5 fold, at least 3 fold, at least 3.5 fold, at least 4 fold, at least 5 fold, at least 6 fold, at least 7 fold, at least 8 fold, at least 9 fold, at least 10 fold, at least 100 fold, at least 1,000 fold, or at least 10,000 fold higher than the amount of activity of an EphB receptor binding peptide as measured by assays well known in the art and/or described herein, e.g., assay to measure affinity binding to the EphB receptor, tyrosine phosphorylation, and cell proliferation. In a specific embodiment, an EphB receptor binding compound or EphB receptor binding peptide has less than 10%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, or less than 95% of the activity of an EphB receptor binding peptide.

In a specific embodiment, an EphB receptor binding compound selectively binds to an EphB receptor with a kon rate of at least 10⁵ M⁻¹s⁻¹, at least 5×10⁵ M⁻¹s⁻¹, at least 10⁶ M⁻¹s⁻¹, at least 5×10⁶M⁻¹s⁻¹, at least 10⁷ M⁻¹s⁻¹, at least 5×10⁷ M⁻¹s⁻¹, or at least 10⁸ M⁻¹s⁻¹.

In another embodiment, an EphB receptor binding compound selectively binds to an EphB receptor with a k_{off} rate of 5×10⁻¹ s⁻¹ or less, 10⁻¹ s⁻¹ or less, 5×10⁻² s⁻¹ or less, 10⁻² s⁻¹ or less, 5×10⁻³ s⁻¹ or less, 10⁻³ s⁻¹ or less, 5×10⁻⁴ s⁻¹ or less, 10⁴ s⁻¹ or less, 5×10⁻⁵ s⁻¹ or less, 10⁻⁵ s⁻¹ or less, 5×10⁻⁶ s⁻¹ or less, 10⁻⁶ s⁻¹ or less, 5×10⁻⁷ s⁻¹ or less, 10⁻⁷ s⁻¹ or less, 5×10⁻⁸ s⁻¹ or less, 10⁻⁸ s⁻¹ or less, 5×10⁻⁹ s⁻¹ or less, 10⁻⁹ s⁻¹ or less, 5×10⁻¹⁰ s⁻¹ or less, or 10⁻¹⁰ s⁻¹ or less.

In another embodiment, an EphB receptor binding compound selectively binds to an EphB receptor with a Kₐ (kₒₙ/k_{off}) of at least 10¹¹ nM⁻¹, at least 5×10¹¹ nM⁻¹, at least 10¹² nM⁻¹, at least 5×10¹² nM⁻¹, at least 10¹³ nM⁻¹, at least 5×10¹³ nM⁻¹, at least 10¹⁴ nM⁻¹, at least 5×10¹⁴ nM⁻¹, at least 10¹⁵ nM⁻¹, at least 5×10¹⁵ nM⁻¹, at least 10¹⁶ nM⁻¹, at least 5×10¹⁶ nM⁻¹, at least 10¹⁷ nM⁻¹, at least 5×10¹⁷ nM⁻¹, at least 10¹⁸ nM⁻¹, at least 5×10¹⁸ nM⁻¹, at least 10¹⁹ nM⁻¹, at least 5×10¹⁹ nM⁻¹, at least 10²⁰ nM⁻¹, at least 5×10²⁰ nM⁻¹, at least 10²¹ nM⁻¹, at least 5×10²¹ nM⁻¹, at least 10²² nM⁻¹, at least 5×10²² nM⁻¹, at least 10²³ nM⁻¹, at least 5×10²³ nM⁻¹, at least 10²⁴ nM⁻¹, or at least 5×10²⁴ nM⁻¹.

In certain embodiments, an EphB receptor binding compound selectively binds to an EphB receptor with a K_{d} (k_{off}/kₒₙ) of 5×10⁷ nM or less, 10⁷ nM or less, 5×10⁶ nM or less, 10⁶ nM or less, 5×10⁵ nM or less, 10⁵ nM or less, 5×10⁴ nM or less, 10⁴ nM or less, 5×10³ nM or less, 10³ nM or less, 5×10² nM or less, 100 nM or less, 90 nM or less, 80 nM or less, 70 nM or less, 60 nM or less, 50 nM or less, 20 nM or less, 15 nM or less, 10 nM or less, 5 nM or less, 3.8 nM or less, 2 nM or less, 1.5 nM or less, 1 nM or less, 5×10⁻¹ nM or less, 10⁻¹ nM or less, 5×10⁻² nM or less, 10⁻² nM or less, 5×10⁻³ nM or less, 10⁻³ nM or less, 5×10⁻⁴ nM or less, 10⁻⁴ nM or less, 5×10⁻⁵ nM or less, 10⁻⁵ nM or less, 5×10⁻⁶ nM or less, or 10⁻⁶ nM or less. In a specific embodiment, an EphB receptor binding compound selectively binds to an EphB receptor with a K_{d} (k_{off}/kₒₙ) between approximately 1 nM and approximately 10 nM, or between approximately 1 nM and approximately 20 nM, or between approximately 1 nM and approximately 30 nM, or between approximately 1 nM and approximately 40 nM, or between approximately 1 nM and approximately 50 nM, or between approximately 1 nM and approximately 60 nM, or between approximately 1 nM and approximately 70 nM, or between approximately 1 nM and approximately 80 nM, or between approximately 1 nM and approximately 90 nM, or between approximately 1 nM and approximately 100 nM, or between approximately 0.1 nM and approximately 100 nM, or between approximately 0.1 nM and approximately 50 nM, or between approximately 0.1 nM and approximately 25 nM, or between approximately 0.1 nM and approximately 10 nM, or between approximately 0.5 nM and approximately 10 nM, or between approximately 0.5 nM and approximately 20 nM, or between approximately 0.5 nM and approximately 250 nM, or between approximately 0.5 nM and approximately 30 nM, or between approximately 0.5 nM and approximately 40 nM, or between approximately 0.5 nM and approximately 50 nM, or between approximately 0.5 nM and approximately 60 nM, or between approximately 0.5 nM and approximately 70 nM, or between approximately 0.5 nM and approximately 80 nM, or between approximately 0.5 nM and approximately 90 nM, or between approximately 0.5 nM and approximately 100 nM, or between approximately 0.5 nM and approximately 5 nM, or between approximately 0.5 nM and approximately 1 nM, or between approximately 10 nM and approximately 100 nM, or between approximately 20 nM and approximately 100 nM, or between approximately 30 nM and approximately 100 nM, or between approximately 40 nM and approximately 100 nM, or between approximately 50 nM and approximately 100 nM, or between approximately 60 nM and approximately 100 nM, or between approximately 70 nM and approximately 100 nM, or between approximately 50 nM and approximately 90 nM, or between approximately 60 nM and approximately 90 nM. In particular embodiments, an EphB receptor binding compound selectively binds to an EphB receptor with a K_{d} (k_{off}/kₒₙ) of between approximately 0.6 nM and approximately 1.1 nM, or between approximately 0.7 nM and approximately 1.2 nM, or between approximately 0.5 and approximately 5 nM. In other specific embodiments, an EphB receptor binding compound selectively binds to an EphB receptor with a K_{d} (k_{off}/kₒₙ) of about 100 nM, about 70 nM, about 60 nM, about 50 nM, about 40 nM, about 30 nM, about 20 nM, about 16 nM, about 15 nM, about 14 nM, about 10 nM, about 5 nM, about 3.5 nM, about 1.2 nM, about 1.1 nM, about 1 nM, about 0.9 nM, about 0.8 nM, about 0.7 nM, or about 0.6 nM as determined using assays known in the art or described herein, e.g., ELISA, isothermal titration calorimetry, or fluorescent polarization assay. In specific embodiments, an EphB receptor binding compound selectively binds to a murine EphB receptor with a K_{d} (k_{off}/kₒₙ) of about 100 nM, about 70 nM, about 60 nM, about 50 nM, about 40 nM, about 30 nM, about 20 nM, about 16 nM, about 15 nM, about 14 nM, about 10 nM, about 5 nM, about 3.5 nM, about 1.2 nM, about 1.1 nM, about 1 nM, about 0.9 nM, about 0.8 nM, about 0.7 nM, or about 0.6 nM as determined using assays known in the art or described herein, e.g., ELISA, isothermal titration calorimetry, or fluorescent polarization assay. In particular embodiments, an EphB receptor binding compound selectively binds to a human EphB receptor with a K_{d} (k_{off}/kₒₙ) of about 100 nM, about 70 nM, about 60 nM, about 50 nM, about 40 nM, about 30 nM, about 20 nM, about 16 nM, about 15 nM, about 14 nM, about 10 nM, about 5 nM, about 3.5 nM, about 1.2 nM, about 1.1 nM, about 1 nM, about 0.9 nM, about 0.8 nM, about 0.7 nM, or about 0.6 nM as determined using assays known in the art or described herein, e.g., ELISA, isothermal titration calorimetry, or fluorescent polarization assay. In particular embodiments, an EphB receptor binding peptide selectively binds to a murine EphB receptor (or an ectodomain of an EphB receptor) with a K_{d} value that is determined by assays well known in the art or described herein, e.g., ELISA, isothermal titration calorimetry (ITC), BIAcore, or fluorescent polarization assay. In particular embodiments, an EphB receptor binding compound selectively binds to a human EphB receptor (or an ectodomain of an EphB receptor) with a K_{d} value that is determined by assays well known in the art or described herein, e.g., ELISA, isothermal titration calorimetry (ITC), BIAcore, or fluorescent polarization assay. In specific embodiments, the K_{d} value of the affinity an EphB receptor binding compound for a murine EphB receptor is measured using assays well known in the art or described herein, e.g., ELISA, isothermal titration calorimetry (ITC), BIAcore, or fluorescent polarization assay. In certain embodiments, the K_{d} value of the affinity an EphB receptor binding compound for a human EphB receptor is measured using assays well known in the art or described herein, e.g., ELISA, isothermal titration calorimetry (ITC), BIAcore, or fluorescent polarization assay. In specific embodiments, the K_{d} value is measured an assay, e.g., by ELISA, isothermal titration calorimetry (ITC), BIAcore, or fluorescent polarization assay, using a preparation of EphB receptor binding compounds that has a purity of approximately 20%, approximately 30%, approximately 40%, approximately 50%, approximately 60%, approximately 70%, approximately 80%, approximately 90%, approximately 95%, or approximately 99%.

In one embodiment, an EphB receptor binding compound selectively binds to an EphB receptor and inhibits the binding of an EphB receptor to an Ephrin B ligand at an IC₅₀ value of less than 5×10⁷ nM, less than 10⁷ nM, less than 5×10⁶ nM, less than 10⁶ nM, less than 5×10⁵ nM, less than 10⁵ nM, less than 5×10⁴ nM, less than 10⁴ nM, less than 5×10³ nM, less than 10³ nM, less than 5×10² nM, less than 100 nM, less than 90 nM, less than 80 nM, less than 70 nM, 69 nM, less than 60 nM, less than 50 nM, less than 40 nM, less than 30 nM, less than 20 nM, less than 15 nM, less than 12 nM, less than 10 nM, less than 5 nm, less than 2 nM, less than 1.5 nM, less than 1 nM, less than 5×10⁻¹ nM, less than 10⁻¹ nM, less than 5×10⁻² nM, less than 10⁻² nM, less than 5×10⁻³ nM, less than 10⁻³ nM, less than 5×10⁻⁴ nM, or less than 10⁻⁴ nM when measured according to methods well known in the art or described herein, e.g., ELISA. In other embodiments, an EphB receptor binding compound selectively binds to EphB4 and inhibits the binding of an EphB4 receptor to an Ephrin B ligand at an IC₅₀ value of between approximately 1 nM and approximately 10 nM, between approximately 1 nM and approximately 15 nM, between approximately 1 nM and approximately 20 nM, between approximately 1 nM and approximately 25 nM, between approximately 1 nM and approximately 30 nM, between approximately 1 nM and approximately 40 nM, between approximately 1 nM and approximately 50 nM, between approximately 1 nM and approximately 60 nM, between approximately 1 nM and approximately 70 nM, between approximately 1 nM and approximately 80 nM, between approximately 1 nM and approximately 90 nM, between approximately 1 nM and approximately 100 nM, between approximately 1 nM and approximately 10 nM, between approximately 1 nM and approximately 10 nM, between approximately 1 nM and approximately 10 nM, between approximately 1 nM and approximately 10 nM, between approximately 1 nM and approximately 10 nM, between approximately 1 nM and approximately 10 nM, between approximately 10 nM and approximately 40 nM, between approximately 10 nM and approximately 50 nM, between approximately 60 nM and approximately 100 nM, between approximately 40 nM and approximately 100 nM, between approximately 50 nM and approximately 100 nM, between approximately 60 nM and approximately 100 nM, between approximately 10 nM and approximately 10² nM, between approximately 10² nM and approximately 10³ nM, between approximately 10³ nM and approximately 10⁴ nM, between approximately 10⁴ nM and approximately 10⁵ nM, between approximately 10⁵ nM and approximately 10⁶ nM, or between approximately 10⁶ nM and approximately 10⁷ nM when measured according to methods well known in the art or described herein, e.g., ELISA. In particular embodiments, an EphB receptor binding compound binds to EphB4 and has an IC₅₀ value of approximately 0.1, 0.5, 1, 5, 6, 7, 8, 9, 10, 11, 12, 13, 14, 15, 16, 17, 18, 19, 20, 25, 30, 40, 50, 60, 70, 80, 90, 100, 110, 120, 130, 140 or 150 nM when measured according to methods well known in the art or described herein, e.g., ELISA. In specific embodiments, an EphB receptor binding compound binds to EphB4 and has an IC₅₀ value of approximately 12 nM, or approximately 14 nM, or approximately 16 nM when measured according to methods well known in the art or described herein, e.g., ELISA. In specific embodiments, an EphB receptor binding compound binds to EphB4 and has an IC₅₀ value of approximately 18 nM, approximately 14 nM, approximately 10 nM, approximately 9 nM, or approximately 7 nM when measured according to methods well known in the art or described herein, e.g., ELISA. In specific embodiments, the IC₅₀ value is determined for an EphB receptor binding compound that inhibits a murine Ephrin B ligand binding to a murine EphB receptor. In particular embodiments, the IC₅₀ is determined for an EphB receptor binding compound peptide that inhibits a murine Ephrin B ligand binding to a human EphB receptor. In some embodiments, the IC₅₀ value is determined for an EphB receptor binding compound that inhibits the binding of a human Ephrin B ligand to a murine EphB receptor. In other embodiments, the IC₅₀ value is determined for an EphB receptor binding compound that inhibits the binding of a human Ephrin B ligand to a human EphB receptor. In specific embodiments, the IC₅₀ value is determined for an EphB receptor binding compound peptide that inhibits the binding of a murine Ephrin B ligand to a murine EphB receptor using ELISA. In specific embodiments, the IC₅₀ value is measured, e.g., by ELISA, using a preparation of EphB receptor binding compounds that has a purity of approximately 20%, approximately 30%, approximately 40%, approximately 50%, approximately 60%, approximately 70%, approximately 80%, approximately 90%, approximately 95%, or approximately 99%. In particular embodiments, an EphB receptor binding compound selectively binds to an EphB receptor and inhibits the binding of an EphB receptor to an Ephrin B ligand at an ICₛₒ value as measured in an assay, e.g., by ELISA, wherein the concentration of soluble Ephrin B ligand or EphB receptor used in the assay is approximately 0.001 μM, 0.005 μM, 0.01 μM, 0.05 μM, 0.1 μM, 0.5 μM, 1 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1000 μM, or 5000 μM.

Table 1, infra, discloses certain peptides that can be used to generate the EphB receptor binding compounds (e.g., peptide dimers, peptide multimers, Fc-fusion peptides and PEG-conjugated peptides, among others). Other EphB receptor binding peptides that can be used to generate the EphB receptor binding compounds include, but are not limited to, fragments of the peptides listed in Table 1, supra, that are at least 5, at least 6, at least 7, at least 8, at least 9, at least 10 or at least 11 amino acid residues in length. In a specific embodiment, EphB receptor binding peptides used to generate the EphB receptor binding compounds include, but are not limited to, fragments of the peptides listed in Table 1, supra, that are between 4 to 5, 4 to 6, 4 to 7, 4 to 8, 4 to 9, 4 to 10, 5 to 10, 6 to 10, 4 to 15, 5 to 15, 6 to 15, 4 to 20, 5 to 20, between 5 to 30, between 5 to 40, between 5 to 50, 6 to 20, 4 to 25, 5 to 25, or 6 to 25 amino acid residues in length. In certain embodiments, EphB receptor binding peptides that can be used to generate the EphB receptor binding compounds have a length of between approximately 4 to approximately 10 amino acid residues, approximately 4 to approximately 15 amino acid residues, approximately 4 to approximately 20 amino acid residues, approximately 4 to approximately 25 amino acid residues, approximately 4 to approximately 30 amino acid residues, approximately 5 to approximately 10 amino acid residues, approximately 5 to approximately 15 amino acid residues, approximately 5 to approximately 20 amino acid residues, approximately 5 to approximately 25 amino acid residues, approximately 5 to approximately 30 amino acid residues, approximately 5 to approximately 40 amino acid residues, approximately 5 to approximately 50 amino acid residues, or approximately 10 to approximately 40 amino acid residues. Still other EphB receptor binding peptides that can be used to generate the EphB receptor binding compounds include, but are not limited to, the peptides listed in Table 1, supra, having at least 1, at least 2, at least 3, at least 4, at least 5, or at least 6 amino acid substitutions, insertions and/or deletions. Included among possible substitutions are conservative substitutions, in which the amino acid sequence is modified by replacing one or more amino acids with different amino acids which have similar chemical or structural characteristics and/or do not significantly alter the biological function of the peptide.

Standard techniques known to those of skill in the art can be used to introduce mutations in the nucleotide sequence encoding a EphB receptor binding peptide, including, for example, site-directed mutagenesis and PCR-mediated mutagenesis which results in amino acid substitutions. In certain embodiments, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a specific embodiment, the derivatives have conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded EphB receptor binding peptide can be inserted into an expression vector and expressed (e.g., in a heterologous host cell) and the activity of the protein can be determined.

In certain embodiments, an EphB receptor binding compound comprises a conjugate. In particular embodiments, the conjugate is a Fc fusion protein. In a specific embodiment, the conjugate is a Fc fusion protein comprising an Fc region of an IgG₁ or a fragment (e.g., CH2 or CH3 domain) thereof. In another specific embodiment, the Fc region is the Fc region of an IgG₂, IgG₃, or IgG₄. In other embodiments, an EphB receptor binding compound comprises a PEG-conjugated EphB receptor binding peptide.

In certain embodiments, an EphB receptor binding compound comprises an EphB receptor binding peptide that is not conjugated to PEG. In specific embodiments, an EphB receptor binding compound does not comprise one EphB receptor binding peptide conjugated to one PEG polymer. In other embodiments, an EphB receptor binding compound comprises an EphB receptor binding peptide that is not conjugated to biotin. In some embodiments, an EphB receptor binding compound comprises an EphB receptor binding peptide that is not conjugated to a therapeutic agent. In another embodiment, an EphB receptor binding compound comprises an EphB receptor binding peptide that is not conjugated to imaging agents. In some other embodiments, an EphB receptor binding compound comprises an EphB receptor binding peptide that is not conjugated to detectable agents. In yet other embodiments, an EphB receptor binding compound comprises an EphB receptor binding peptide that is not conjugated to diagnostic agents.

In some embodiments, an EphB receptor binding compound comprises a multimeric peptide. In other embodiments, an EphB receptor binding compound comprises a multimeric peptide comprising 2, 3, 4, 5, 6, 7, 8, 9, 10 or more EphB receptor binding peptides. The EphB receptor binding peptides that make up the multimeric peptide can be 1, 2 or more of the peptides in Table 1. In certain embodiments, the EphB receptor binding compound comprises a multimeric peptide that is homomeric, i.e., contains two or more peptides, each of which comprises the same amino acid sequence. In certain embodiments, the EphB receptor binding compound comprises a multimeric peptide which is heteromeric, i.e., contains two or more peptides which have different amino acid sequences. In certain embodiments, the EphB receptor binding compound comprises a multimeric peptide which is a homodimer, i.e., contains two peptides of the same amino acid sequence. In certain embodiments, the EphB receptor binding compound comprises a multimeric peptide which is a heterodimer, i.e., contains two peptides of different amino acid sequences. In certain embodiments, the EphB receptor binding compound comprises a PEG-linked peptide homodimer of the amino acid sequence TNYLFSPNGPIARAW, (“TNYL-RAW,” SEQ ID NO:39), e.g., (TNYL-RAW)₂-PEG. In other embodiments, the EphB receptor binding compound comprises a PEG-linked peptide homodimer of the amino acid sequence NYLFSPNGPIARAW, (“NYL-RAW,” SEQ ID NO:40), e.g., (NYL-RAW)₂-PEG. In some embodiments, the EphB receptor binding compound comprises a PEG-linked peptide homodimer of the amino acid sequence YLFSPNGPIARAW, (“YL-RAW,” SEQ ID NO:41), e.g., (YL-RAW)₂—PEG.

TABLE 1
EphB Receptor-Binding Peptides

| | | | | EphB Receptor | | | | | |
|---|---|---|---|---|---|---|---|---|---|
| | | | | Specificity | | | | | |
| Panning | | SEQ | | (% Binding)^{c} | | | | | |
| Receptor | # clones^{a} | ID NO | Peptide Sequence^{b} | B1 | B2 | B3 | B4 | B6 | Peptide IC₅₀^{d} |

| | | | | | | | | | |
|---|---|---|---|---|---|---|---|---|---|
| EphB1 | 1⁴ | 1 | EWLSPNLAPSVR | 100 | 2 | 1 | 1 | 0 | ~10 μM^{e} |
| | 2³ | 2 | TTLSQLPKSTWL | 100 | 0 | 1 | 0 | 0 | nd |
| | 16^{3,4} | 3 | AHTFPYPHPKPH | 100 | 4 | 8 | 1 | 0 | ~150 μM |
| | 1³ | 4 | SHKFPGPPSWMS | 88 | 100 | 10 | 9 | 0 | nd |
| | 1³ | 5 | THWKFQPWALVT | 86 | 100 | 5 | 0 | 0 | nd |
| EphB2 | 1^{3.2} | 6 | THWCHLLNCAAL | 100 | 89 | 85 | 20 | 26 | nd |
| | 1^{3.2} | 7 | WHRYPDPRMLPT | 54 | 100 | 31 | 13 | 15 | nd |
| | 5^{3.2,4} | 8 | WHWTIEPFAITS | 99 | 100 | 22 | 20 | 45 | >100 μM |
| | 1² | 9 | DHWYYTPWQPIE | 100 | 81 | 5 | 5 | 2 | nd |
| | 2^{2,3.2} | 10 | DHWRILPFSLSS | 53 | 100 | 4 | 1 | 3 | >100 μM |
| | 1² | 11 | IHWPVAPYSYLD | 97 | 100 | 4 | 4 | 7 | nd |
| | 1² | 12 | SHWPVLPFAHWQ | 98 | 100 | 9 | 6 | 10 | nd |
| | 5^{2,3.2,4} | 13 | DHWRVSPYSLLY | 100 | 97 | 1 | 0 | 1 | nd |
| | 1⁴ | 14 | SHWPISPYSLLS | 39 | 100 | 9 | 4 | 8 | >100 μM |
| | 1² | 15 | NHWPTQPYAIPI | 18 | 100 | 22 | 22 | 0 | nd |
| | 1⁴ | 16 | DHWPLLPYALALH | 4 | 100 | 7 | 0 | 0 | nd |
| | 1² | 17 | WPPHWPRSLDYA | 0 | 100 | 0 | 0 | 0 | nd |
| | 3² | 18 | SNEWIQPRLPQH | 1 | 100 | 7 | 1 | 12 | ~15 μM^{e} |
| EphB4 | 1^{3.2} | 19 | EWYMKFPPEHYF | 22 | 23 | 23 | 100 | 30 | >600 μM |
| | 7^{3(1st)} | 20 | DALNDWLLFRPW | 0 | 0 | 10 | 100 | 16 | >100 μM |
| | 4^{3.1,3.4,4} | 21 | DHNHNLYNPWRL | 9 | 8 | 9 | 100 | 7 | ~200 μM |
| | 1² | 22 | TYFDFQAWSIRA | 10 | 21 | 18 | 100 | 30 | >50 μM^{f} |
| | 1^{3.2} | 23 | EFFTWRPTYYGI | 16 | 12 | 29 | 100 | 17 | nd |
| | 5^{2,3.1,4} | 24 | TNYLFSPNGPIA | 5 | 3 | 3 | 100 | 7 | ~50 μM^{g} |
| | 1⁴ | 25 | FSPQGPAARNFA | 13 | 8 | 11 | 100 | 11 | >600 μM |
| | 1² | 26 | LPHGPVAAAWDA | 2 | 15 | 2 | 100 | 4 | nd |
| | 1^{3.2} | 27 | NPVIGPIQRAWT | 1 | 0 | 4 | 100 | 0 | >500 μM |
| | 1^{3.1} | 28 | SHVGPIMRAWAP | 22 | 6 | 2 | 100 | 6 | nd |
| | 1^{3.1} | 29 | GPVHRAWEPTSH | 4 | 6 | 16 | 100 | 7 | nd |
| | 2^{3.2,4} | 30 | GPVERAWRPDLI | 1 | 2 | 1 | 100 | 3 | nd |
| | 1^{3.1} | 31 | GPVSKAWQETET | 13 | 13 | 14 | 100 | 16 | nd |
| | 2³ | 32 | GPVADAWLVYPR | 10 | 5 | 4 | 100 | 6 | nd |
| | 1^{3.1} | 33 | WGIPRAAQVMWT | 16 | 17 | 20 | 100 | 14 | nd |
| | 1^{3(1st)} | 34 | IPWTQHMANSPM | nd | nd | nd | 100 | nd | nd |
| | 1^{3(1st)} | 35 | SGHQLLLNKMPN | nd | nd | nd | 100 | nd | nd |
| Ephrin-B1 | | 36 | KFQEFSPNYMGLEFKKHHDY | | | | | | |
| Ephrin-B2 | | 37 | KFQEFSPNLWGLEFQKNKDY | | | | | | |
| Ephrin-B3 | | 38 | KFQEYSPNLWGHEFRSHHDY | | | | | | |
| EphB4 | | 39 | TNYLFSPNGPIARAW | | | | | | See below |
| EphB4 | | 40 | NYLFSPNGPIARAW | | | | | | See below |
| EphB4 | | 41 | YLFSPNGPIARAW | | | | | | See below |
| EphB4 | | 42 | LFSPNGPIARAW | | | | | | See below |
| EphB4 | | 43 | DALNDWLLFRPW | | | | | | |
| EphB4 | | 44 | IPWTQHMAMSPM | | | | | | |
| EphB4 | | 45 | SVSVGMKPSPRP | | | | | | |
| EphB4 | | 46 | SGHQLLLNKMPN | | | | | | |
| EphB4 | | 47 | GPVADAWLVYPR | | | | | | |
| EphB4 | | 48 | NPVIGPIQRAWT | | | | | | |
| EphB4 | | 49 | DHNHDLYNPWRL | | | | | | |
| EphB4 | | 50 | TNYLFSPNGPIA | | | | | | |
| EphB4 | | 51 | LPHGPVAAAWDA | | | | | | |
| EphB4 | | 52 | TYFDFQAWSIRA | | | | | | |
| EphB4 | | 53 | EWYMKFPPEHYF | | | | | | |
| EphB4 | | 54 | GPVHRAWEPTSH | | | | | | |
| EphB4 | | 55 | SHVGPIMRAWAP | | | | | | |
| EphB4 | | 56 | WGIPRAAQVMWT | | | | | | |
| EphB4 | | 57 | GPVSKAWQETET | | | | | | |
| EphB4 | | 58 | EFFTWRPTYYGI | | | | | | |
| EphB4 | | 59 | GPVERAWRPDLI | | | | | | |
| EphB4 | | 60 | DHNHNLYNPWRL | | | | | | |
| EphB4 | | 61 | FSPQGPAARNFA | | | | | | |
| EphB2 | | 62 | SHWPISPYSLLS | | | | | | |
| EphB2 | | 63 | DHWRVSPYSLLY | | | | | | |
| EphB2 | | 64 | SNEWIQPRLPQH | | | | | | |
| EphB2 | | 65 | DHWRILPFSLSS | | | | | | |
| EphB2 | | 66 | SHWPVLPFAHWQ | | | | | | |
| EphB2 | | 67 | IHWPVAPYSYLD | | | | | | |
| EphB2 | | 68 | WHRYPDPRMLPT | | | | | | |
| EphB2 | | 69 | WHWTIEPFAITS | | | | | | |
| EphB2 | | 70 | THWCHLLNCAAL | | | | | | |
| EphB2 | | 71 | DHWYYTPWQPIE | | | | | | |
| EphB2 | | 72 | NHWPTQPYAIPI | | | | | | |
| EphB2 | | 73 | WPPHWPRSLDYA | | | | | | |
| EphB2 | | 74 | DHWPLLPYALAH | | | | | | |
| EphB2 | | 75 | RNKRIRMQLPMI | | | | | | |

^{a}The round of panning is indicated as a superscript, 1st is the first panning on EphB4.
^{b}The alignment was by eye, and the G-H loop of the human ephrins are underlined.
^{c}Values >25 are indicated in bold.
^{d}Concentration of peptide required to inhibit ephrin-B2 AP binding by 50%; IC₅₀ values with “>” before the concentration indicates either the concentration required to inhibit ephrin-B2 AP binding by 50% or the highest concentration tested that did not inhibit ephrin-B2 AP binding by 50%.
^{e}Biotinylated and non-biotinylated peptides gave the same result.
^{f}Limited by solubility
^{g}This value was obtained with the biotinylated peptide, although the IC₅₀ for the non-biotinylated peptide is 150 μM.
nd, not determined.

5.1.1 Preparation of EphB Receptor Binding Peptides

The EphB receptor binding peptides described herein can be prepared by standard methods known in the art, for example, by solid phase synthesis and recombinant DNA/genetic engineering technology. Non-limiting examples of solid phase synthesis include exclusive solid phase synthesis, partial solid phase synthesis methods, fragment condensation, and classical solution synthesis. See, for example, Merrifield, 1963 J Am Chem Soc 85:2149, which is incorporated herein by reference. For EphB receptor binding peptides produced by recombinant/genetic engineering techniques, nucleic acids which encode the EphB receptor binding peptides can be inserted into various expression vectors and introduced into heterologous host cells for recombinant expression. For a standard protocols of recombinant DNA techniques, see, e.g., Sambrook and Russell, 2001, Molecular Cloning: A Laboratory Manual, 3^{rd} ed. Cold Spring Harbor Laboratory Press (Cold Spring Harbor, N.Y. 2001), and U.S. Patent Publication No. US 2004/0091486 A1 (see, e.g., paragraph numbers 136-148) and references cited therein, each of which is incorporated herein by reference. In specific embodiments, the purity of a preparation of EphB receptor binding compounds or EphB receptor binding peptides is approximately 20%, approximately 30%, approximately 40%, approximately 50%, approximately 60%, approximately 70%, approximately 80%, approximately 90%, approximately 95%, or approximately 99% using assays well known in the art and/or described herein.

5.1.1.1 Solid Synthesis

On solid phase, the synthesis is typically commenced from the C-terminal end of the peptide using an alpha-amino protected resin. A suitable starting material can be prepared, for instance, by attaching the required alpha-amino acid to a chloromethylated resin, a hydroxymethyl resin, or a benzhydrylamine resin. One such chloromethylated resin is sold under the trade name BIO-BEADS SX-1 by BioRad Laboratories, Richmond, Calif., and the preparation of the hydroxymethyl resin is described by Bodonszky, et al. 1966 Chem Ind (London) 38:1597. The benzhydrylamine (BHA) resin has been described by Pietta and Marshall, 1970 Chem Commun 650, and is commercially available from Beckman Instruments, Inc., Palo Alto, Calif., in the hydrochloride form.

Thus, compounds can be prepared by coupling an alpha-amino protected amino acid to the chloromethylated resin with the aid of, for example, cesium bicarbonate catalyst, according to the method described by Gisin, 1973 Helv Chim Acta 56:1467. After the initial coupling, the alpha-amino protecting group is removed by a choice of reagents including trifluoroacetic acid (TFA) or hydrochloric acid (HCl) solutions in organic solvents at room temperature.

The alpha-amino protecting groups are those known to be useful in the art of stepwise synthesis of peptides. Included are acyl type protecting groups (for example, formyl, trifluoroacetyl, acetyl), aromatic urethane type protecting groups (for example benzyloxycarboyl (Cbz) and substituted Cbz), aliphatic urethane protecting groups (for example, t-butyloxycarbonyl (Boc), isopropyloxycarbonyl, cyclohexyloxycarbonyl) and alkyl type protecting groups (for example, benzyl, triphenylmethyl). Boc and Fmoc are preferred protecting groups. The side chain protecting group remains intact during coupling and is not split off during the deprotection of the amino-terminus protecting group or during coupling. The side chain protecting group must be removable upon the completion of the synthesis of the final peptide and under reaction conditions that will not alter the target peptide.

The side chain protecting groups for Tyr include tetrahydropyranyl, tert-butyl, trityl, benzyl, Cbz, Z—Br-Cbz, and 2,5-dichlorobenzyl. The side chain protecting groups for Asp include benzyl, 2,6-dichlorobenzyl, methyl, ethyl, and cyclohexyl. The side chain protecting groups for Thr and Ser include acetyl, benzoyl, trityl, tetrahydropyranyl, benzyl, 2,6-dichlorobenzyl, and Cbz. The side chain protecting group for Thr and Ser is benzyl. The side chain protecting groups for Arg include nitro, Tosyl (Tos), Cbz, adamantyloxycarbonyl mesitoylsulfonyl (Mts), or Boc. The side chain protecting groups for Lys include Cbz, 2-chlorobenzyloxycarbonyl (2Cl-Cbz), 2-bromobenzyloxycarbonyl (2-BrCbz), Tos, or Boc.

After removal of the alpha-amino protecting group, the remaining protected amino acids are coupled stepwise in the desired order. An excess of each protected amino acid is generally used with an appropriate carboxyl group activator such as dicyclohexylcarbodiimide (DCC) in solution, for example, in methylene chloride (CH₂Cl₂), dimethyl formamide (DMF) mixtures.

These procedures can also be used to synthesize peptides in which amino acids other than the 20 naturally occurring, genetically encoded amino acids are substituted at one, two, or more positions of any of the compounds. For instance, naphthylalanine can be substituted for tryptophan, facilitating synthesis. Other synthetic amino acids that can be substituted into the peptides of the present invention include L-hydroxypropyl, L-3,4-dihydroxy-phenylalanyl, d amino acids such as L-d-hydroxylysyl and D-d-methylalanyl, L-α-methylalanyl, β amino acids, and isoquinolyl. D amino acids and non-naturally occurring synthetic amino acids can also be incorporated into the peptides of the present invention (see, for example, Roberts, et al. 1983 Unusual Amino/Acids in Peptide Synthesis 5:341-449).

After the desired amino acid sequence has been completed, the desired peptide is decoupled from the resin support by treatment with a reagent such as trifluoroacetic acid or hydrogen fluoride (HF), which not only cleaves the peptide from the resin, but also cleaves all remaining side chain protecting groups. When the chloromethylated resin is used, hydrogen fluoride treatment results in the formation of the free peptide acids. When the benzhydrylamine resin is used, hydrogen fluoride treatment results directly in the free peptide amide. Alternatively, when the chloromethylated resin is employed, the side chain protected peptide can be decoupled by treatment of the peptide resin with ammonia to give the desired side chain protected amide or with an alkylamine to give a side chain protected alkylamide or dialkylamide. Side chain protection is then removed in the usual fashion by treatment with hydrogen fluoride to give the free amides, alkylamides, or dialkylamides.

These solid phase peptide synthesis procedures are well known in the art and further described by J. M. Stewart and J. D. Young, 1984 Solid Phase Peptide Syntheses 2nd Ed., Pierce Chemical Company.

Using the “encoded synthetic library” or “very large scale immobilized polymer synthesis” system described in U.S. patent application Ser. No. 07/492,462, filed Mar. 7, 1990; Ser. No. 07/624,120, filed Dec. 6, 1990; and Ser. No. 07/805,727, filed Dec. 6, 1991; one can not only determine the minimum size of a peptide with such activity, one can also make all of the peptides that form the group of peptides that differ from the preferred motif (or the minimum size of that motif) in one, two, or more residues. This collection of peptides can then be screened for ability to bind to members of the Eph receptor family including, but not limited to, EphB1, EphB2, EphB3, EphB4, EphB5 and EphB6. It will be appreciated that this immobilized polymer synthesis system or other peptide synthesis methods can also be used to synthesize truncation analogs and deletion analogs and combinations of truncation and deletion analogs of the EphB receptor binding peptides.

5.1.1.2 Peptide Modifications

A variety of techniques are available for modifying an EphB receptor binding peptide to prepare another EphB receptor binding peptide (e.g., an analog or derivative) with the same desired biological activity as the unmodified peptide but with more favorable activity with respect to solubility, stability, and/or susceptibility to hydrolysis and proteolysis. See, for example, Morgan, et al. 1989 Ann Rep Med Chem 24:243-252. The following describes methods for preparing EphB receptor binding peptides which are modified at the N-terminal amino group, at the C-terminal carboxyl group, at other reactive sites (e.g., side chains) on the amino acid residues of the peptide, and/or by changing one or more of the amido linkages in the peptide to a non-amido linkage. It is understood that two or more such modifications can be coupled in one EphB receptor binding peptide (for example, modification at the C-terminal carboxyl group and inclusion of a —CH₂-carbamate linkage between two amino acids in the peptide).

(A) N-Terminal Modifications

The EphB receptor binding peptides typically are synthesized as the free acid but could be readily prepared as the amide or ester. One can also modify the amino and/or carboxy terminus of the peptide to produce EphB receptor binding peptides. Amino terminus modifications include methylation (i.e., —NHCH₃ or —NH(CH₃)₂), acetylation, adding a benzyloxycarbonyl group, or blocking the amino terminus with any blocking group containing a carboxylate functionality defined by RCOO—, where R is selected from the group consisting of naphthyl, acridinyl, steroidyl, and similar groups. Carboxy terminus modifications include replacing the free acid with a carboxamide group or forming a cyclic lactam at the carboxy terminus to introduce structural constraints.

Amino terminus modifications are as recited above and include alkylating, acetylating, adding a carbobenzoyl group, forming a succinimide group, etc. (See, for example, Murray, et al. 1995 Burger's Medicinal Chemistry and Drug Discovery 5th ed., Vol. 1, Manfred E. Wolf, ed., John Wiley and Sons, Inc.). Specifically, the N-terminal amino group can then be reacted as follows:

(a) to form an amide group of the formula RC(O)NH— where R is as defined above by reaction with an acid halide [for example, RC(O)Cl] or symmetric anhydride. Typically, the reaction can be conducted by contacting about equimolar or excess amounts (for example, about 5 equivalents) of an acid halide to the peptide in an inert diluent (for example, dichloromethane) preferably containing an excess (for example, about 10 equivalents) of a tertiary amine, such as diisopropylethylamine, to scavenge the acid generated during reaction. Reaction conditions are otherwise conventional (for example, room temperature for 30 minutes). Alkylation of the terminal amino to provide for a lower alkyl N-substitution followed by reaction with an acid halide as described above will provide for N-alkyl amide group of the formula RC(O)NR—;

(b) to form a succinimide group by reaction with succinic anhydride. As before, an approximately equimolar amount or an excess of succinic anhydride (for example, about 5 equivalents) can be employed and the amino group is converted to the succinimide by methods well known in the art including the use of an excess (for example, ten equivalents) of a tertiary amine such as diisopropylethylamine in a suitable inert solvent (for example, dichloromethane). See, for example, Wollenberg, et al., U.S. Pat. No. 4,612,132 which is incorporated herein by reference in its entirety. It is understood that the succinic group can be substituted with, for example, C₂-C₆ alkyl or —SR substituents which are prepared in a conventional manner to provide for substituted succinimide at the N-terminus of the peptide. Such alkyl substituents are prepared by reaction of a lower olefin (C₂-C₆) with maleic anhydride in the manner described by Wollenberg, et al., supra and —SR substituents are prepared by reaction of RSH with maleic anhydride where R is as defined above;

(c) to form a benzyloxycarbonyl-NH— or a substituted benzyloxycarbonyl-NH— group by reaction with approximately an equivalent amount or an excess of CBZ-Cl (i.e., benzyloxycarbonyl chloride) or a substituted CBZ-Cl in a suitable inert diluent (for example, dichloromethane) preferably containing a tertiary amine to scavenge the acid generated during the reaction;

(d) to form a sulfonamide group by reaction with an equivalent amount or an excess (for example, 5 equivalents) of R—S(O)₂Cl in a suitable inert diluent (dichloromethane) to convert the terminal amine into a sulfonamide where R is as defined above. Preferably, the inert diluent contains excess tertiary amine (for example, ten equivalents) such as diisopropylethylamine, to scavenge the acid generated during reaction. Reaction conditions are otherwise conventional (for example, room temperature for 30 minutes);

(e) to form a carbamate group by reaction with an equivalent amount or an excess (for example, 5 equivalents) of R—OC(O)Cl or R—OC(O)OC₆H₄-p-NO₂ in a suitable inert diluent (for example, dichloromethane) to convert the terminal amine into a carbamate where R is as defined above. Preferably, the inert diluent contains an excess (for example, about 10 equivalents) of a tertiary amine, such as diisopropylethylamine, to scavenge any acid generated during reaction. Reaction conditions are otherwise conventional (for example, room temperature for 30 minutes); and

(f) to form a urea group by reaction with an equivalent amount or an excess (for example, 5 equivalents) of R—N═C═O in a suitable inert diluent (for example, dichloromethane) to convert the terminal amine into a urea (i.e., RNHC(O)NH—) group where R is as defined above. Preferably, the inert diluent contains an excess (for example, about 10 equivalents) of a tertiary amine, such as diisopropylethylamine. Reaction conditions are otherwise conventional (for example, room temperature for about 30 minutes).

(B) C-Terminal Modifications

In preparing EphB receptor binding peptides wherein the C-terminal carboxyl group is replaced by an ester (i.e., —C(O)OR where R is as defined above), the resins used to prepare the peptide acids are employed, and the side chain protected peptide is cleaved with base and the appropriate alcohol, for example, methanol. Side chain protecting groups are then removed in the usual fashion by treatment with hydrogen fluoride to obtain the desired ester.

In preparing EphB receptor binding peptides wherein the C-terminal carboxyl group is replaced by the amide —C(O)NR³R⁴, a benzhydrylamine resin is used as the solid support for peptide synthesis. Upon completion of the synthesis, hydrogen fluoride treatment to release the peptide from the support results directly in the free peptide amide (i.e., the C-terminus is —C(O)NH₂). Alternatively, use of the chloromethylated resin during peptide synthesis coupled with reaction with ammonia to cleave the side chain protected peptide from the support yields the free peptide amide and reaction with an alkylamine or a dialkylamine yields a side chain protected alkylamide or dialkylamide (i.e., the C-terminus is —C(O)NRR₁ where R and R¹ are as defined above). Side chain protection is then removed in the usual fashion by treatment with hydrogen fluoride to give the free amides, alkylamides, or dialkylamides.

In another alternative embodiment, the C-terminal carboxyl group or a C-terminal ester can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Such methods are well known in the art.

One can also cyclize the peptides, or incorporate a desamino or descarboxy residue at the termini of the peptide, so that there is no terminal amino or carboxyl group, to decrease susceptibility to proteases or to restrict the conformation of the peptide. C-terminal functional groups of the compounds of the present invention include amide, amide lower alkyl, amide di(lower alkyl), lower alkoxy, hydroxy, and carboxy, and the lower ester derivatives thereof, and the pharmaceutically acceptable salts thereof.

(C) Backbone Modifications

Methods which can be used for making EphB receptor binding peptides of the present invention are described in Hruby, et al. 1990 Biochem J 268(2):249-262, incorporated herein by reference. EphB receptor binding peptides also serve as structural models for non-peptidic compounds with similar biological activity. Those of skill in the art recognize that a variety of techniques are available for modifying EphB receptor binding peptides to produce other EphB receptor binding peptides with the same or similar desired biological activity as unmodified EphB receptor binding peptides but with more favorable activity than the lead with respect to solubility, stability, and susceptibility to hydrolysis and proteolysis. See Morgan, et al. 1989 Ann Rep Med Chem 24:243-252, incorporated herein by reference. These techniques include replacing the peptide backbone with a backbone composed of phosphonates, amidates, carbamates, sulfonamides, secondary amines, and N-methylamino acids.

EphB receptor binding peptides wherein one or more of the peptidyl linkages [—C(O)NH—] have been replaced by such linkages as a —CH₂-carbamate linkage, a phosphonate linkage, a —CH₂-sulfonamide linkage, a urea linkage, a secondary amine (—CH₂NH—) linkage, an alkylated peptidyl linkage [—C(O)NR⁶— where R⁶ is lower alkyl], or other linkages such as, but not limited to, —CH₂S—, —CH₂CH₂—, —CH═CH— (cis and trans), —COCH₂—, —CH(OH)CH₂—, and CH₂SO—, are prepared according to methods known in the art, e.g., during conventional peptide synthesis a suitably protected amino acid analog is substituted for the amino acid reagent at the appropriate point during synthesis.

Suitable reagents include, for example, amino acid analogs wherein the carboxyl group of the amino acid has been replaced with a moiety suitable for forming one of the above linkages. For example, if one desires to replace a —C(O)NR— linkage in the peptide with a —CH₂-carbamate linkage (—CH₂OC(O)NR—), then the carboxyl (—COOH) group of a suitably protected amino acid is first reduced to the —CH₂OH group which is then converted by conventional methods to a OC(O)Cl functionality or a para-nitrocarbonate —OC(O)O—C₆H₄—P—NO₂ functionality. Reaction of either of such functional groups with the free amine or an alkylated amine on the N-terminus of the partially fabricated peptide found on the solid support leads to the formation of a —CH₂OC(O)NR— linkage. For a more detailed description of the formation of such —CH₂-carbamate linkages, see Cho, et al. 1993 Science 261:1303-1305.

Similarly, replacement of an amido linkage in the peptide with a phosphonate linkage can be achieved in the manner set forth in U.S. patent application Ser. Nos. 07/943,805, 08/081,577, and 08/119,700, the disclosures of which are incorporated herein by reference in their entirety.

Replacement of an amido linkage in the peptide with a —CH₂-sulfonamide linkage can be achieved by reducing the carboxyl (—COOH) group of a suitably protected amino acid to the —CH₂OH group and the hydroxyl group is then converted to a suitable leaving group such as a tosyl group by conventional methods. Reaction of the tosylated derivative with, for example, thioacetic acid followed by hydrolysis and oxidative chlorination will provide for the —CH₂—S(O)₂Cl functional group which replaces the carboxyl group of the otherwise suitably protected amino acid. Use of this suitably protected amino acid analog in peptide synthesis provides for inclusion of a —CH₂S(O)₂NR— linkage, which replaces the amido linkage in the peptide thereby providing an analog, derivative or peptidomimetic. For a more complete description on the conversion of the carboxyl group of the amino acid to a —CH₂S(O)₂Cl group, see, for example, Weinstein, B., 1983 Chemistry & Biochemistry of Amino Acids, Peptides and Proteins Vol. 7, pp. 267-357, Marcel Dekker, Inc., New York, which is incorporated herein by reference.

Replacement of an amido linkage in the peptide with a urea linkage can be achieved in the manner set forth in U.S. patent application Ser. No. 08/147,805 which application is incorporated herein by reference in its entirety.

Secondary amine linkages wherein a —CH₂NH— linkage replaces the amido linkage in the peptide can be prepared by employing, for example, a suitably protected dipeptide analog wherein the carbonyl bond of the amido linkage has been reduced to a CH₂ group by conventional methods. For example, in the case of diglycine, reduction of the amide to the amine will yield after deprotection H₂NCH₂CH₂NHCH₂COOH which is then used in N-protected form in the next coupling reaction. The preparation of such analogs by reduction of the carbonyl group of the amido linkage in the dipeptide is well known in the art (see, for example, M. W. Remington 1994 Meth Mol Bio 35:241-247).

The suitably protected amino acid analog is employed in the conventional peptide synthesis in the same manner as would the corresponding amino acid. For example, typically about 3 equivalents of the protected amino acid analog are employed in this reaction. An inert organic diluent such as methylene chloride or DMF is employed and, when an acid is generated as a reaction by-product, the reaction solvent will typically contain an excess amount of a tertiary amine to scavenge the acid generated during the reaction. One particularly preferred tertiary amine is diisopropylethylamine which is typically employed in about 10-fold excess. The reaction results in incorporation into the analog, derivative or peptidomimetic of an amino acid analog having a non-peptidyl linkage. Such substitution can be repeated as desired such that from zero to all of the amido bonds in the peptide have been replaced by non-amido bonds.

One can also cyclize the peptides, or incorporate a desamino or descarboxy residue at the termini of the peptide, so that there is no terminal amino or carboxyl group, to decrease susceptibility to proteases or to restrict the conformation of the peptide. C-terminal functional groups of the compounds include amide, amide lower alkyl, amide di(lower alkyl), lower alkoxy, hydroxy, and carboxy, and the lower ester derivatives thereof, and the pharmaceutically acceptable salts thereof.

(D) Disulfide Bond Formation

The EphB receptor binding peptides may exist in a cyclized form with an intramolecular disulfide bond between the thiol groups of the cysteines. Other embodiments of this invention include analogs of these disulfide derivatives in which one of the sulfurs has been replaced by a CH₂ group or other isostere for sulfur. These analogs can be made via an intramolecular or intermolecular displacement, using methods known in the art.

Alternatively, the amino-terminus of the peptide can be capped with an alpha-substituted acetic acid, wherein the alpha substituent is a leaving group, such as an α-haloacetic acid, for example, α-chloroacetic acid, α-bromoacetic acid, or α-iodoacetic acid. The compounds of the present invention can be cyclized or dimerized via displacement of the leaving group by the sulfur of the cysteine or homocysteine residue. See, for example, Andreu, et al. 1994 Meth Mol Bio 35(7):91-169; Barker, et al. 1992 J Med Chem 35:2040-2048; and Or, et al. 1991 J Org Chem 56:3146-3149, each of which is incorporated herein by reference.

(E) Additional Chemical Modifications

In certain embodiments, the EphB receptor binding peptides may be subjected to chemical modifications to produce other EphB receptor binding compounds. The chemical modification may be performed in addition to other modifications described herein. In certain embodiments, chemical modifications result in the addition of functional groups to the peptides. The chemical modifications may be performed at any point of reactivity on the peptide, including, but not limited to, the N-terminus, C-terminus, and/or a reactive side chain on the peptide.

One can replace the naturally occurring side chains of the 20 genetically encoded amino acids (or D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclic. In particular, proline analogs in which the ring size of the proline residue is changed from 5 members to 4, 6, or 7 members can be employed. Cyclic groups can be saturated or unsaturated, and if unsaturated, can be aromatic or non-aromatic. Heterocyclic groups preferably contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl (for example, morpholino), oxazolyl, piperazinyl (for example, 1-piperazinyl), piperidyl (for example, 1-piperidyl, piperidino), pyranyl, pyrazinyl, pyrazolidinyl, pyrazolinyl, pyrazolyl, pyridazinyl, pyridyl, pyrimidinyl, pyrrolidinyl (for example, 1-pyrrolidinyl), pyrrolinyl, pyrrolyl, thiadiazolyl, thiazolyl, thienyl, thiomorpholinyl (for example, thiomorpholino), and triazolyl. These heterocyclic groups can be substituted or unsubstituted. Where a group is substituted, the substituent can be alkyl, alkoxy, halogen, oxygen, or substituted or unsubstituted phenyl.

In certain embodiments, the EphB receptor binding peptides described herein are modified to include the addition of chemical functional groups. In certain embodiments, the peptide is chemically modified to add one or more acetyl groups by way of an acetylation reaction. In other embodiments, the peptide is chemically modified to add one or more alkyl groups, e.g., methyl or ethyl groups, by way of an alkylation reaction, such as, e.g., the addition of a methyl group to a lysine and/or arginine residue. In other embodiments, the peptide is chemically modified to add one or more biotin appendages. In other embodiments, the peptide is chemically modified to add one or more phosphate groups by way of a phosphorylation reaction, such as, e.g., the addition of a phosphate group to a serine, tyrosine, threonine and/or histidine residue (see, for example, W. Bannwarth, et al. 1996 Biorganic and Medicinal Chemistry Letters 6:2141-2146). In other embodiments, the peptide is chemically modified to add one or more sulfate groups, such as, e.g., the addition of a sulfate group to a tyrosine residue. In other embodiments, the peptide is chemically modified to add one or more saccharides by way of a glycosylation reaction, such as, e.g., the addition of a glycosyl group to an asparagine, hydroxylysine, serine and/or threonine residue. Other methods which can be employed for chemically modifying the EphB receptor binding peptides are described in Hruby, et al. 1990 Biochem J 268:249-262.

5.1.2 Preparation of EphB Receptor Binding Compounds

In certain embodiments, the EphB receptor binding peptides described above can be conjugated (e.g., fused or linked) with one or more peptides (e.g., those described in Table 1, supra), analogs, derivatives, peptidomimetics, antibody fragments (e.g., Fc antibody fragments), heterologous compounds, marker sequences and/or therapeutic agents to provide EphB receptor binding compounds which have improved properties, such as, e.g., improved selectivities, potency, binding affinities and/or improved half-lives.

In certain embodiments, EphB receptor binding compounds do not comprise an EphB receptor binding peptide covalently coupled to one or more hydrophilic polymers. In some embodiments, EphB receptor binding compounds do not comprise an EphB receptor binding peptide covalently coupled to PEG. In specific embodiments, EphB receptor binding compounds do not comprise an EphB receptor binding peptide covalently coupled to unbranched PEG. In some embodiments, EphB receptor binding compounds do not comprise an EphB receptor binding peptide covalently coupled to branched PEG. In other embodiments, EphB receptor binding compounds do not comprise an EphB receptor binding peptide covalently coupled to PEG having a molecular weight ranging from 5,000 daltons to about 20,000 daltons. In some embodiments, EphB receptor binding compounds do not comprise one EphB receptor binding peptide covalently coupled to one PEG. In particular embodiments, EphB receptor binding compounds do not consist essentionally of one EphB receptor binding peptide covalently coupled to one PEG.

In some embodiments, EphB receptor binding compounds do not comprise an EphB receptor binding peptide covalently coupled to cellulose or cellulose derivatives. In specific embodiments, EphB receptor binding compounds do not comprise an EphB receptor binding peptide covalently coupled to dextran or dextran derivatives. In other embodiments, EphB receptor binding compounds do not comprise an EphB receptor binding peptide covalently coupled to biotin.

In certain embodiments, EphB receptor binding compounds do not comprise an EphB receptor binding peptide covalently coupled to polypropylene glycol, polylactic acid, polyglycolic acid, polyoxyalkenes, polyvinylalcohol, or polyvinylpyrrolidone. In some embodiments, EphB receptor binding compounds do not comprise an EphB receptor binding peptide covalently coupled to therapeutic agents. In other embodiments, EphB receptor binding compounds do not comprise an EphB receptor binding peptide covalently coupled to detectable agents. In certain embodiments, EphB receptor binding compounds do not comprise an EphB receptor binding peptide covalently coupled to diagnostic agents.

In some embodiments, EphB receptor binding compounds do not comprise an EphB receptor binding peptide covalently coupled to a peptide or chemical linker. In particular embodiments, EphB receptor binding compounds do not comprise an EphB receptor binding peptide with N-terminal modifications. In some embodiments, EphB receptor binding compounds do not comprise an EphB receptor binding peptide with C-terminal modifications.

5.1.2.1 Peptide Conjugation

The present invention encompasses multimeric (including homomultimeric and heteromultimeric) EphB receptor binding compounds comprising 1 or more, 2 or more, 3 or more, 4 or more, 5 or more, 6 or more, 7 or more, 8 or more, 9 or more, 10 or more, 11 or more, or 12 or more EphB receptor binding peptides. EphB receptor binding compounds may be prepared by conjugating peptides by the methods described herein. EphB receptor binding compounds may exist as dimers, in which two peptides are conjugated, or higher conjugates, in which three or more peptides are conjugated. The EphB receptor binding compounds may comprise more than one peptide with the same amino acid sequence (homomultimeric compounds) and/or may contain peptides with different amino acid sequences (heteromultimeric compounds). In certain embodiments, the number of peptides conjugated together in the EphB receptor binding compounds is less than 12, less than 11, less than 10, less than 9, less than 8, less than 7, less than 6, less than 5, less than 4 or less than 3 peptides. In specific embodiments, 2 peptides, 3 peptides, 4 peptides, 5 peptides, 6 peptides, 7 peptides, 8 peptides, 9 peptides, 10 peptides, 11 peptides or 12 peptides are conjugated together to form an EphB receptor binding compound.

In certain embodiments, one or more EphB receptor binding peptides comprising at least 4, at least 5, at least 6, at least 7 at least 8, at least 9, at least 10, at least 20, at least 30, or at least 40 amino acid residues are conjugated to form the EphB receptor binding compounds. In other embodiments, one or more EphB receptor binding peptides comprising less than 11, less than 21, less than 31, less than 41, or less than 50 amino acid residues are conjugated to form the EphB receptor binding compounds. In certain embodiments, one or more EphB receptor binding peptides comprising between approximately 4 to approximately 10, approximately 5 to approximately 10, approximately 6 to approximately 10, approximately 4 to approximately 15, approximately 5 to approximately 15, approximately 6 to approximately 15, approximately 4 to approximately 12, approximately 5 to approximately 12, approximately 6 to approximately 12, approximately 10 to approximately 15, approximately 10 to approximately 20, approximately 10 to approximately 30, approximately 10 to approximately 40, approximately 10 to approximately 50, approximately 15 to approximately 25, approximately 20 to approximately 30, approximately 25 to approximately 35, approximately 30 to approximately 40, approximately 30 to approximately 50, or approximately 40 to approximately 50 amino acid residues are conjugated to form the EphB receptor binding compounds. In a specific embodiment, one or more EphB receptor binding peptides comprising 10, 12, or 15 amino acid residues are conjugated to form the EphB receptor binding compounds. In one embodiment, the EphB receptor binding compounds result from conjugating two or more of the peptides of SEQ ID NOS:1-75, as listed in Table 1. In other embodiments, the EphB receptor binding compounds result from conjugating two or more peptide analogs or derivatives with structures based upon the peptides of SEQ ID NOS:1-75, as listed in Table 1. In other embodiments, the EphB receptor binding compounds result from conjugating together peptides of SEQ ID NOS:1-75, as listed in Table 1 with analogs, derivatives or heterologous peptides together in one EphB receptor binding compound. In other embodiments, the EphB receptor binding compounds result from conjugating two or more EphB receptor binding peptides of the same amino acid sequence together in one homomultimeric EphB receptor binding compound.

Specific embodiments provide EphB receptor binding compounds which are peptide dimers resulting from the conjugation of two peptides with sequences selected from the group consisting of SEQ ID NOS:1-75 in Table 1. One embodiment provides a peptide dimer resulting from the conjugation (including fusion or chemical linkage, as described herein) of two peptides of the amino acid sequence TNYLFSPNGPIARAW (“TNYL-RAW”), corresponding to SEQ ID NO:39 in Table 1. In another embodiment, the present invention provides a peptide dimer resulting from the conjugation (including fusion or chemical linkage, as described herein) of two peptides of the amino acid sequence NYLFSPNGPIARAW (“NYL-RAW”), corresponding to SEQ ID NO:40 in Table 1. In another embodiment, the present invention provides a peptide dimer resulting from the conjugation (including fusion or chemical linkage, as described herein) of two peptides of the amino acid sequence YLFSPNGPIARAW (“YL-RAW”), corresponding to SEQ ID NO:41 in Table 1. In another embodiment, the present invention provides a dimer resulting from the conjugation (including fusion or chemical linkage, as described herein) of two peptides of an amino acid sequence selected from the amino acid sequences of SEQ ID NOS:1-75 in Table 1. Other embodiments provide a dimer resulting from the conjugation (including fusion or chemical linkage, as described herein) of two peptides of two different amino acid sequences selected from the amino acid sequences of SEQ ID NOS:1-75 in Table 1. In certain embodiments, the peptide dimers described herein bind to an EphB receptor (e.g., EphB1, EphB2, EphB3, EphB4, EphB5 or EphB6) with higher affinity than non-dimerized or monomeric peptides. In a specific embodiment, the peptide dimers described herein bind to the EphB4 receptor with higher affinity than non-dimerized or monomeric peptides.

In other embodiments, the EphB receptor binding compounds are formed by the conjugation (including fusion and/or chemical linkage) of three or more peptides or modified peptides, including analogs and/or derivatives thereof, into a multimeric EphB receptor binding compound. In certain embodiments, the multimeric EphB receptor binding compounds include one or more peptides of amino acid sequences selected from the amino acid sequences of SEQ ID NOS:1-75 in Table 1. In certain embodiments, the multimeric EphB receptor binding compounds described herein bind to an EphB receptor (e.g., EphB1, EphB2, EphB3, EphB4, EphB5 or EphB6) with higher affinity than non-multimeric or monomeric peptides. In a specific embodiment, the multimeric EphB receptor binding compounds described herein bind to the EphB4 receptor with higher affinity than non-multimeric or monomeric peptides.

The dimeric and higher multimeric EphB receptor binding compounds can be conjugated by directly fusing two or more components (e.g., via peptide bonds) and/or can be conjugated by chemically linking two or more components using chemical linkers, as described infra. In certain embodiments, peptides are conjugated into dimeric EphB receptor binding compounds using polyethylene glycol (PEG) or a chemical derivative thereof, as described in detail infra. In specific embodiments, two amino acid sequences selected from the group consisting of TNYL-RAW, NYL-RAW and YL-RAW (SEQ ID NOS:39, 40, and 41, respectively, as shown in Table 1) are conjugated as dimeric EphB receptor binding compounds using a bifunctional PEG or a chemical derivative of PEG. In specific embodiments, the two peptides which are linked together have identical amino acid sequences. In a specific embodiment, the PEG-linked dimeric EphB receptor binding compound binds to an EphB receptor (e.g., EphB1, EphB2, EphB3, EphB4, EphB5 or EphB6) with higher affinity than a non-dimerized peptide of the same sequence. In particular embodiments, the EphB receptor is murine EphB receptor. In certain embodiments, the EphB receptor is human EphB receptor.

In particular embodiments, the conjugation efficiency of two or more EphB receptor binding peptides conjugated to PEG or a chemical derivative thereof to form EphB receptor binding compounds is approximately 10%, approximately 20%, approximately 30%, approximately 40%, approximately 50%, approximately 60%, approximately 70%, approximately 80%, approximately 85%, approximately 90%, approximately 95%, or approximately 100% as determined by assays well known in the art, e.g., conjugated and unconjugated fractions are separated and quantitation of the fractions can be determined by, e.g., ELISA or spectrometry. Assays well known in the art or described herein, e.g., affinity chromatography and HPLC, can be performed to separate the fractions of conjugated and unconjugated peptides and PEG.

In certain embodiments, the multimeric EphB receptor binding compounds are agonistic. In other embodiments, the multimeric EphB receptor binding compounds are antagonistic.

5.1.2.2 Linkers

The EphB receptor binding peptides that make up the multimeric EphB receptor binding compounds described supra may be conjugated together directly (i.e., fused) using non-covalent or covalent bonds, e.g., peptide bonds. In other embodiments, EphB receptor binding peptides are conjugated together by using one or more chemical linkers, disulfide bonds, Fc regions of an IgG or fragments (e.g., CH2 or CH3 domain) thereof and/or other chemical moieties capable of joining together two or more amino acids.

In certain embodiments, the linker that joins the EphB receptor binding peptides exhibits conformational flexibility. In other embodiments, the length of the linker that joins the peptides is optimized to maximize binding affinity to an EphB receptor (e.g., EphB1, EphB2, EphB3, EphB4, EphB5 or EphB6).

In some embodiments, peptide linkers are used to join the EphB receptor binding peptides to make up the multimeric EphB receptor binding compounds. In certain embodiments, the peptide linkers are made up of a majority of amino acids that are sterically unhindered, such as glycine and alanine. Certain peptide linkers include poly-glycines, poly-alanines, and peptides comprising alanine and glycine. In one embodiment, the peptide linker is 1, 2, 3, 4, 5, 6, 7, 8, 9, 10, 15 or 20 amino acid residues in length. In other embodiments, the peptide linker is between approximately 1 to approximately 5, approximately 5 to approximately 10, approximately 2 to approximately 6, or approximately 3 to approximately 7 amino acid residues in length. In one embodiment, the EphB receptor binding compounds does not comprise a linker.

In other embodiments, an intermolecular disulfide bond between the thiol groups of amino acids (e.g., cysteines) can yield a dimeric or higher multimeric EphB receptor binding compound, including homomeric and heteromeric compounds. One or more of the cysteine residues may also be substituted with a homocysteine. In certain embodiments, peptides may be modified to specifically include one or more thiol-containing amino acids, e.g., cysteine, which may participate in disulfide bond formation in order to provide EphB receptor binding compounds. In a specific embodiment, the dimeric or higher multimeric EphB receptor binding compounds, including homomeric and heteromeric compounds are not produced using disulfide bonds.

In other embodiments, non-peptide linkers are used to join the EphB receptor binding peptides to make up the EphB receptor binding compounds. In some embodiments, linkers comprising covalently bonded alkyl groups are used to form the EphB receptor binding compounds. An alkyl linker can, for example, take the form of —NH—(CH₂)ₙ—C(O)—, where n is an integer number of methylene units. Alkyl linkers may be linear or branched and/or may be saturated or unsaturated. Other embodiments include the use of alkyl linkers which are substituted by one or more chemical functional groups, including, but not limited to, alkyl, acyl, halogen, nitrile, amino, phenyl, ether and the like.

In other embodiments, non-peptide polymers are used to join the EphB receptor binding peptides to make up the multimer compounds. In certain embodiments, the non-peptide polymer is a hydrophilic polymer. Polymers which may be employed as linkers in the multimer compounds include multifunctional versions of polyalkylethers, as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives, and the like. In certain embodiments, such hydrophilic polymers have an average molecular weight in the ranges of about 500 to about 100,000 daltons, about 2,000 to about 40,000 daltons, or about 3,000 to about 20,000 daltons. In other embodiments, such hydrophilic polymers have average molecular weights of about 3,000 daltons, 4,000 daltons, 5,000 daltons, 10,000 daltons and 20,000 daltons.

The EphB receptor binding compounds can be derivatized with or coupled to such polymers using any methods known in the art, including the methods set forth in Zallipsky, S. 1995 Bioconjugate Chem 6:150-165; Monfardini, C, et al. 1995 Bioconjugate Chem 6:62-69; U.S. Pat. Nos. 4,640,835; 4,496,689; 4,301,144; 4,670,417; 4,791,192; 4,179,337 or WO 95/34326, all of which are incorporated by reference in their entirety herein.

In certain embodiments, the peptides are linked together with multifunctional polyethylene glycol (PEG), including functionally-modified PEG. PEGs are classified by their molecular weights which typically range from about 500 daltons to about 40,000 daltons. In certain embodiments, the PEGs employed have molecular weights ranging from 5,000 daltons to about 20,000 daltons. The PEGs used as linkers in the present invention can be either branched or unbranched, and can be bifunctional or multifunctional. (See, for example, Monfardini, C. et al. 1995 Bioconjugate Chem 6:62-69). PEGs, including bifunctional PEGs, are commercially available from Nektar Therapeutics AL, Co. (Huntsville, Ala.), Sigma Chemical Co., and other companies. Such bifunctional or multifunctional PEGs include, but are not limited to: PEGs suitable for conjugation to amines, such as, e.g., polyethylene glycol-bis-(succinimidyl α-methylbutanoate) (SMB-PEG-SMB) and polyethylene glycol-bis-(carboxymethyl-3-hydroxybutanoate-N-hydroxyl-succinimidyl ester) (NHS-HBA-CM-PEG-CM-HBA-NHS); PEGs suitable for conjugation to the N-terminal of a peptide, such as, e.g., polyethylene glycol-bis-buteraldehyde (ButyrALD-PEG-ButyrALD); PEGs suitable for conjugation to thiols, such as, e.g., polyethylene glycol-bis-maleimide (MAL-PEG-MAL), monomethoxypolyethylene glycol-bis-forked-maleimide (mPEG-MAL2), branched-monomethoxypolyethylene glycol-bis-forked maleimide (mPEG2-MAL2) and polyethylene glycol-bis-ortho-pyridyldisulfide (OPSS-PEG-OPSS); and PEGs suited for forming conjugates between two different polypeptides, such as, e.g., 9-fluorenylmethoxycarbonyl-polyethylene glycol-N-hydroxy-succinimidyl ester (Fmoc-PEG-NHS), tert-butyloxycarbonyl-polyethylene glycol-N-hydroxy-succinimidyl ester (Boc-PEG-NHS), and other heterobifunctional derivatives of PEG which have the general structure X-PEG-Y, where X and Y represent two different PEG-activating functional groups. Such linkers are available commercially, e.g., from Nektar Therapeutics AL, Co. (Huntsville, Ala.).

In one non-limiting exemplar embodiment, the hydrophilic polymer which is employed as a linker is a bifunctional PEG which has a reactive functional group at each end, e.g., a cyanuric halide (for example, cyanuric chloride, bromide or fluoride), an anhydride reagent (for example, a dihalosuccinic anhydride, such as dibromosuccinic anhydride), acyl azide, p-diazoniumbenzyl ether, 3-(p-diazoniumphenoxy)-2-hydroxypropylether), an imide derivative (for example, succinimidyl propionate or N-hydroxysuccinimide), a thiol derivative, or the like. The activated polymer is then reacted with an EphB receptor binding peptide using methods described herein or using normal PEGylation conditions as known in the art (e.g., for amine PEGylation, an amide bond can be formed between the protein and the peptide) to produce a PEG-linked EphB receptor binding multimeric compound. Alternatively, a functional group in the EphB receptor binding peptides can be activated for reaction with the polymer, or the two groups can be joined in a concerted coupling reaction using known coupling methods. It will be readily appreciated that the EphB receptor binding peptides can be derivatized with PEG using a myriad of other reaction schemes known to and used by those of skill in the art.

In certain embodiments, the chemical linker that links the peptides to form the dimeric or multimeric EphB receptor binding compounds of the present invention is selected such that its length is optimized to provide advantageous binding affinity. Methods of optimizing linker length to provide advantageous binding affinity are known in the art, and are described in detail, e.g., in Kramer and Karpen, Nature (1998) 395:710-713. In particular embodiments, the conjugation efficiency of two or more EphB receptor binding peptides conjugated to a chemical linker to form EphB receptor binding compounds is approximately 10%, approximately 20%, approximately 30%, approximately 40%, approximately 50%, approximately 60%, approximately 70%, approximately 80%, approximately 85%, approximately 90%, approximately 95%, or approximately 100% as determined by assays well known in the art, e.g., conjugated and unconjugated fractions are separated and quantitation of the fractions can be determined by, e.g., ELISA or spectrometry. Assays well known in the art or described herein, e.g., affinity chromatography and HPLC, can be performed to separate the unconjugated and conjugated fractions.

Specific embodiments described herein provide EphB receptor binding compounds resulting from the conjugation of two peptides of SEQ ID NOS:1-75, as listed in Table 1, joined by a chemical linker as described herein. In some embodiments, an EphB receptor binding compound results from the conjugation of two peptides of the amino acid sequence TNYLFSPNGPIARAW, corresponding to SEQ ID NO:39 in Table 1, linked together by a PEG polymer of approximately 3.4 kDa. In other embodiments, an EphB receptor binding compound results from the conjugation of two peptides of the amino acid sequence TNYLFSPNGPIARAW, corresponding to SEQ ID NO:39 in Table 1, linked together by a PEG polymer of approximately 10 kDa. In specific embodiments, an EphB receptor binding compound results from the conjugation of two peptides of the amino acid sequence TNYLFSPNGPIARAW, corresponding to SEQ ID NO:39 in Table 1, linked together by a PEG polymer of approximately 40 kDa. In certain embodiments, an EphB receptor binding compound results from the conjugation of two peptides of the amino acid sequence NYLFSPNGPIARAW, corresponding to SEQ ID NO:40 in Table 1, linked together by a PEG polymer of approximately 3.4 kDa. In particular embodiments, the present invention provides an EphB receptor binding compound resulting from the conjugation of two peptides of the amino acid sequence NYLFSPNGPIARAW, corresponding to SEQ ID NO:40 in Table 1, linked together by a PEG polymer of approximately 10 kDa. In specific embodiments, the present invention provides an EphB receptor binding compound resulting from the conjugation of two peptides of the amino acid sequence NYLFSPNGPIARAW, corresponding to SEQ ID NO:40 in Table 1, linked together by a PEG polymer of approximately 40 kDa. In certain embodiments, the present invention provides an EphB receptor binding compound resulting from the conjugation of two peptides of the amino acid sequence YLFSPNGPIARAW, corresponding to SEQ ID NO:41 in Table 1, linked together by a PEG polymer of approximately 3.4 kDa. In other embodiments, the present invention provides an EphB receptor binding compound resulting from the conjugation of two peptides of the amino acid sequence YLFSPNGPIARAW, corresponding to SEQ ID NO:41 in Table 1, linked together by a PEG polymer of approximately 10 kDa. In particular embodiments, the present invention provides an EphB receptor binding compound resulting from the conjugation of two peptides of the amino acid sequence YLFSPNGPIARAW, corresponding to SEQ ID NO:41 in Table 1, linked together by a PEG polymer of approximately 40 kDa. In certain embodiments, the EphB receptor binding compound peptide dimers described herein bind to EphB4 with higher affinity than non-dimerized or monomeric peptides.

5.1.2.3 Conjugation with Antibody Fragments

In certain embodiments, the present invention provides EphB receptor binding compounds comprising an EphB receptor binding peptide conjugated (e.g., fused or chemically linked) to an antibody fragment (in certain embodiments, antibody fragments of at least 10, at least 20, at least 30, at least 40, at least 50, at least 60, at least 70, at least 80, at least 90 or at least 100 amino acids are used) to generate multimeric compounds or EphB receptor binding compounds. The components may be conjugated by direct fusion or may be conjugated through linker amino acid sequences or other linkers, such as, e.g., hydrophilic polymers, as described herein. Antibody fragments may be used to target peptides to particular cell types, either in vitro or in vivo, by conjugating (e.g., fusing or chemically linking) the peptides to antibody fragments specific for particular cell surface receptors. EphB receptor binding peptides conjugated to antibody fragments may also be used to modify the half-life of a peptide, or to modify the degradation profile of a peptide. EphB receptor binding peptides conjugated to antibody fragments may also be used in in vitro immunoassays and purification methods using methods known in the art. See e.g., International Publication WO 93/21232; EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452, which are incorporated by reference in their entireties. In one embodiment, an EphB receptor binding compound comprises one or more EphB receptor binding peptides conjugated to a whole antibody. In a certain embodiment, EphB receptor binding compounds do not comprise an EphB receptor binding peptide conjugated to a whole antibody. In particular embodiments, the conjugation efficiency of two or more EphB receptor binding peptides conjugated to antibody fragments to form EphB receptor binding compounds is approximately 10%, approximately 20%, approximately 30%, approximately 40%, approximately 50%, approximately 60%, approximately 70%, approximately 80%, approximately 85%, approximately 90%, approximately 95%, or approximately 100% as determined by assays well known in the art, e.g., conjugated and unconjugated fractions are separated and quantitation of the fractions can be determined by, e.g., ELISA or spectrometry.

In specific embodiments, EphB receptor binding compounds comprise an EphB receptor binding peptide conjugated (e.g., fused or chemically linked) to a native Fc region of an IgG immunoglobulin or a fragment (e.g., CH2 or CH3 domain) thereof. A native Fc region is a molecule or sequence comprising the non-antigen-binding fragment resulting from digestion of whole antibody, whether in monomeric or multimeric form. The original immunoglobulin source of the native Fc is, in certain embodiments, of human origin and may be any of the immunoglobulins. In certain embodiments, IgG1 and IgG2 are used. Native Fc regions are made up of monomeric polypeptides that may be linked into dimeric or multimeric forms by covalent bonds (e.g., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgA2). One example of a native Fc region is a disulfide-bonded dimer resulting from papain digestion of an IgG (see Ellison et al. (1982), Nucleic Acids Res., 10:4071-9). The term “native Fc” as used herein is generic to the monomeric, dimeric and multimeric forms. A Fc variant is a molecule or sequence that is modified from a native Fc but still comprises a binding site for the salvage receptor, FcRn. International applications WO 97/34631 and WO 96/32478 describe exemplary Fc variants, as well as interaction with the salvage receptor, and are hereby incorporated by reference. Thus, Fc variants may include a molecule or sequence that is humanized from a non-human native Fc. Furthermore, a native Fc comprises sites that may be removed because they provide structural features or biological activity that are not required for the fusion molecules of the present invention. Thus, Fc variants may include a molecule or sequence that lacks one or more native Fc sites or residues that affect or are involved in (1) disulfide bond formation, (2) incompatibility with a selected host cell, (3) N-terminal homogeneity upon expression in the host cell, (4) glycosylation, (5) interaction with complement, (6) binding to an Fc receptor other than a salvage receptor, or (7) antibody-dependent cellular cytotoxicity (ADCC). A Fc fragment and/or Fc variant may be conjugated (e.g., directly fused or joined via a chemical linker) to the EphB receptor binding peptide at one or more of the N-terminus, C-terminus, or a side chain of one or more of the amino acid residues. In certain embodiments, the addition of a native Fc fragment or a Fc variant to the peptide introduces advantageous properties. In certain embodiments, the addition of a native Fc region of an IgG or a Fc variant provides an increased half-life and/or improves the peptide degradation profile. In particular embodiments, the addition of a native Fc region or Fc variant increases the half-life of EphB receptor binding peptides by approximately 0.5 fold, approximately 1 fold, approximately 1.5 fold, approximately 2 fold, approximately 2.5 fold, approximately 3 fold, approximately 3.5 fold, approximately 4 fold, approximately 4.5 fold, approximately 5 fold, approximately 5.5 fold, approximately 6 fold, approximately 6.5 fold, approximately 7 fold, approximately 8 fold, approximately 9 fold, approximately 10 fold, approximately 15 fold, approximately 20 fold, approximately 30 fold, approximately 40 fold, approximately 50 fold, approximately 60 fold, approximately 70 fold, approximately 80 fold, approximately 90 fold, approximately 100 fold, approximately 500 fold or approximately 1000 fold, as determined by assays known in the art or described herein.

In certain embodiments, the EphB receptor binding compounds contain EphB receptor binding peptides which are not conjugated to antibody fragments, including Fc fragments or Fc variants. In specific embodiments, the EphB receptor binding compound comprising one or more EphB receptor binding peptide conjugated to an Fc region or Fc variant further comprises a peptide linker having the amino acid sequence: Gly Ser Gly Ser Lys (SEQ ID NO:76). In one embodiment, an EphB receptor binding compound comprises one or more EphB receptor binding peptide conjugated to an Fc region or Fc variant, wherein the EphB receptor binding compound is an agonist. In certain embodiments, an EphB receptor binding compound comprises one or more EphB receptor binding peptide conjugated to an Fc region or Fc variant, wherein the EphB receptor binding compound is an antagonist. In particular embodiments, the conjugation efficiency of two or more EphB receptor binding peptides conjugated to an Fc region or Fc variant to form EphB receptor binding compounds is approximately 10%, approximately 20%, approximately 30%, approximately 40%, approximately 50%, approximately 60%, approximately 70%, approximately 80%, approximately 85%, approximately 90%, approximately 95%, or approximately 100% as determined by assays well known in the art, e.g., conjugated and unconjugated fractions are separated and quantitation of the fractions can be determined by, e.g., ELISA or spectrometry. Assays well known in the art or described herein, e.g., affinity chromatography and HPLC, can be performed to separate the fractions of conjugated and unconjugated peptides and PEG. Assays well known in the art or described herein, e.g., affinity chromatography and HPLC, can be performed to separate the unconjugated and conjugated fractions.

5.1.2.4 Conjugation with Polymers

In certain embodiments, the EphB receptor binding peptides (e.g., the peptides listed in Table 1) can be covalently or non-covalently coupled to one or more heterologous compounds, e.g., hydrophilic polymers. When the EphB receptor binding peptides are derivatized with a hydrophilic polymer, their solubility and circulation half-lives can be increased, their biological distribution patterns can be improved, and their immunogenicity can be masked, thereby offering protection from biological degradation. Advantageously, the foregoing can be accomplished with little, if any, diminishment in their binding activity. In certain embodiments, EphB receptor binding compounds do not comprise an EphB receptor binding peptide conjugated to a hydrophilic polymers. In particular embodiments, the conjugation efficiency of two or more EphB receptor binding peptides conjugated to heterologous compounds to form EphB receptor binding compounds is approximately 10%, approximately 20%, approximately 30%, approximately 40%, approximately 50%, approximately 60%, approximately 70%, approximately 80%, approximately 85%, approximately 90%, approximately 95%, or approximately 100% as determined by assays well known in the art, e.g., conjugated and unconjugated fractions are separated and quantitation of the fractions can be determined by, e.g., ELISA or spectrometry. Assays well known in the art or described herein, e.g., affinity chromatography and HPLC, can be performed to separate the unconjugated and conjugated fractions.

Nonproteinaceous polymers suitable for use include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives, and the like. In certain embodiments, such hydrophilic polymers have an average molecular weight in the ranges of about 500 to about 100,000 daltons, about 2,000 to about 40,000 daltons, or about 5,000 to about 20,000 daltons. In other embodiments, such hydrophilic polymers have average molecular weights of about 5,000 daltons, 10,000 daltons and 20,000 daltons.

The EphB receptor binding peptides can be derivatized with or coupled to such polymers using any of the methods set forth in Zallipsky, S. 1995 Bioconjugate Chem 6:150-165; Monfardini, C, et al. 1995 Bioconjugate Chem 6:62-69; U.S. Pat. Nos. 4,640,835; 4,496,689; 4,301,144; 4,670,417; 4,791,192; 4,179,337 or WO 95/34326, all of which are incorporated by reference in their entirety herein.

In certain embodiments, the EphB receptor binding peptides are derivatized with polyethylene glycol (PEG), including functionally-modified PEG. PEG is a linear, water-soluble polymer of ethylene oxide repeating units with two terminal hydroxyl groups. PEGs are classified by their molecular weights which typically range from about 500 daltons to about 40,000 daltons. In certain embodiments, the PEGs employed have molecular weights ranging from 5,000 daltons to about 20,000 daltons. PEGs coupled to the EphB receptor binding peptides of the present invention can be either branched or unbranched. (See, for example, Monfardini, C. et al. 1995 Bioconjugate Chem 6:62-69). PEGs, including functionally-modified PEGs, are commercially available from Nektar Therapeutics AL, Co. (Huntsville, Ala.), Sigma Chemical Co. and other companies. Such PEGs include, but are not limited to, monomethoxypolyethylene glycol (MePEG-OH), monomethoxypolyethylene glycol-succinate (MePEG-S), monomethoxypolyethylene glycol-N-hydroxy-succinimide (MePEG-NHS) monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), monomethoxypolyethylene glycol-imidazolyl-carbonyl (MePEG-IM), monomethoxypolyethylene glycol-succinimidyl α-methylbutanoate (MePEG-SMG), monomethoxypolyethylene glycol-succinimidyl propionate (MePEG-SPA), monomethoxypolyethylene glycol-carboxymethyl-3-hydroxybutanoate-N-hydroxy-succinimide (MePEG-CM-HBA-NHS), monomethoxypolyethylene glycol-acetaldehyde, monomethoxypolyethylene glycol-propionaldehyde, monomethoxypolyethylene glycol-butyraldehyde (MePEG-ButyrALD), monomethoxypolyethylene glycol-ortho-pyridylthioester (MePEG-OPTE), monomethoxypolyethylene glycol-maleimide (MePEG-MAL), monomethoxypolyethylene glycol-ortho-pyridyldisulfide (MePEG-OPSS), and monomethoxypolyethylene glycol-thiol (MePEG-SH).

Briefly, in one exemplar embodiment, the hydrophilic polymer which is employed, for example, PEG, is preferably capped at one end by an unreactive group such as a methoxy or ethoxy group. Thereafter, the polymer is activated at the other end by reaction with a suitable activating agent, such as cyanuric halides (for example, cyanuric chloride, bromide or fluoride), an anhydride reagent (for example, a dihalosuccinic anhydride, such as dibromosuccinic anhydride), acyl azide, p-diazoniumbenzyl ether, 3-(p-diazoniumphenoxy)-2-hydroxypropylether), an imide derivative, a thiol derivative, and the like. The activated polymer is then reacted with an EphB receptor binding peptide as described herein or as known in the art to produce a polymer-derivitized EphB receptor binding compound. Alternatively, a functional group in the EphB receptor binding peptides can be activated for reaction with the polymer, or the two groups can be joined in a concerted coupling reaction using known coupling methods. It will be readily appreciated that the EphB receptor binding peptides can be derivatized with PEG using a myriad of other reaction schemes known to and used by those of skill in the art.

Peptides suitable for use in this embodiment generally include the EphB receptor binding peptides described herein, e.g., the peptides of SEQ ID NOS: 1-75, as listed in Table 1. Hydrophilic polymers suitable for use in the present invention include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives, etc. In certain embodiments, such hydrophilic polymers have an average molecular weight ranging from about 500 to about 100,000 daltons, from about 2,000 to about 40,000 daltons, or from about 5,000 to about 20,000 daltons. In some embodiments, such hydrophilic polymers have average molecular weights of about 5,000 daltons, 10,000 daltons or 20,000 daltons.

In some embodiments, EphB receptor binding compounds comprise an EphB receptor binding peptide that is not conjugated to one or more hydrophilic polymers. In other embodiments, EphB receptor binding compounds comprise an EphB receptor binding peptide that is not conjugated to PEG. In certain embodiments, EphB receptor binding compounds comprise an EphB receptor binding peptide that is not conjugated to unbranched PEG. In specific embodiments, EphB receptor binding compounds comprise an EphB receptor binding peptide that is not conjugated to branched PEG. In other embodiments, EphB receptor binding compounds comprise an EphB receptor binding peptide that is not conjugated to PEG having a molecular weight ranging from 5,000 daltons to about 20,000 daltons. In particular embodiments, EphB receptor binding compounds comprise an EphB receptor binding peptide that is not conjugated to PEG having a molecular weight of 3.4 kDa or 10 kDa. In some embodiments, EphB receptor binding compounds comprise an EphB receptor binding peptide that is not conjugated to PEG. In other embodiments, EphB receptor binding compounds comprise two or more EphB receptor binding peptides that are not conjugated to PEG. In specific embodiments, EphB receptor binding compounds comprise an EphB receptor binding peptide that is not conjugated to cellulose or cellulose derivatives. In some embodiments, EphB receptor binding compounds comprise an EphB receptor binding peptide that is not conjugated to dextran or dextran derivatives. In certain embodiments, EphB receptor binding compounds comprise an EphB receptor binding peptide that is not conjugated to one or more polymers selected from the group consisting of polypropylene glycol, polylactic acid, polyglycolic acid, polyoxyalkenes, polyvinylalcohol, and polyvinylpyrrolidone.

5.1.2.5 Conjugation with Marker Sequences

In certain embodiments, EphB receptor binding compounds can be formed by conjugating EphB receptor binding peptides described herein to marker sequences, e.g., another peptide, to facilitate purification. In specific embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, PNAS 86:821, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In certain embodiments, EphB receptor binding compounds do not comprise an EphB receptor binding peptide conjugated to one or more marker sequences. In specific embodiments, EphB receptor binding compounds do not comprise an EphB receptor binding peptide conjugated to one or more “HA” tag. In certain embodiments, EphB receptor binding compounds do not comprise an EphB receptor binding peptide conjugated to one or more “flag” tag. In other embodiments, EphB receptor binding compounds do not comprise an EphB receptor binding peptide conjugated to one or more hexa-histidine.

In other embodiments, the EphB receptor binding peptides described herein are conjugated to a diagnostic or detectable agent. Such conjugates can be useful for monitoring or prognosing the development or progression of a cancer as part of a clinical testing procedure, such as determining the efficacy of a particular therapy. Additionally, such conjugates can be useful for monitoring or prognosing the development or progression of a pre-cancerous condition associated with cells that overexpress an EphB receptor.

Such diagnosis and detection can accomplished by coupling the EphB receptor binding peptides to detectable substances including, but not limited to various enzymes, such as but not limited to, horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga) germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵_{Rh}), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

In one embodiment, EphB receptor binding compounds comprise multimeric EphB receptor binding peptides, wherein one or more of the EphB receptor binding peptides are conjugated to a detectable agent or marker sequence. In specific embodiments, EphB receptor binding compounds comprise one or more EphB receptor binding peptides conjugated to an Fc region that is conjugated to a detectable agent or marker sequence. In certain embodiments, the EphB receptor binding compounds can be used to detect or diagnose an EphB receptor related disease or disorder.

In certain embodiments, the EphB receptor binding compounds contain EphB receptor binding peptides that are not conjugated to marker sequences or to diagnostic agents or to detectable agents. In other embodiments, EphB receptor binding compounds comprise an EphB receptor binding peptide that is not conjugated to biotin. In specific embodiments, EphB receptor binding compounds comprise EphB receptor binding peptides that are not conjugated to ¹²⁵I. In other embodiments, EphB receptor binding compounds comprise EphB receptor binding peptides that are not conjugated to fluorescent materials, such as but not limited to, umbelliferone, fluorescein, fluorescein isothiocynate, rhodamine, dichlorotriazinylamine fluorescein, dansyl chloride or phycoerythrin. In specific embodiments, EphB receptor binding compounds comprise EphB receptor binding peptides that are not conjugated to a compound selected from the group consisting of horseradish peroxidase, alkaline phosphatase, beta-galactosidase, and acetylcholinesterase. In other embodiments, EphB receptor binding compounds comprise EphB receptor binding peptides that are not conjugated to bioluminescent materials, such as but not limited to, luciferase, luciferin, and aequorin. In specific embodiments, EphB receptor binding compounds comprise EphB receptor binding peptides that are not conjugated to radioactive materials.

5.1.2.6 Conjugation with Therapeutic Agents

In certain embodiments, the present invention provides EphB receptor binding peptides conjugated (e.g., fused or chemically linked) to a therapeutic agent to form an EphB receptor binding compound. One or more peptides may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, a peptide may be conjugated (e.g., fused or chemically linked) to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Moreover, a peptide can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the peptide via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50 each incorporated by reference in their entireties. In particular embodiments, the conjugation efficiency of two or more EphB receptor binding peptides conjugated to therapeutic agents to form EphB receptor binding compounds is approximately 10%, approximately 20%, approximately 30%, approximately 40%, approximately 50%, approximately 60%, approximately 70%, approximately 80%, approximately 85%, approximately 90%, approximately 95%, or approximately 100% as determined by assays well known in the art, e.g., conjugated and unconjugated fractions are separated and quantitation of the fractions can be determined by, e.g., ELISA or spectrometry. Assays well known in the art or described herein, e.g., affinity chromatography and HPLC, can be performed to separate the unconjugated and conjugated fractions.

In certain embodiments, the EphB receptor binding compounds contain EphB receptor binding peptides that are not conjugated to therapeutic agents or drug moieties. In other embodiments, the EphB receptor binding compounds are conjugates that are not conjugated to therapeutic agents or drug moieties. In particular embodiments, EphB receptor binding compounds comprise multimeric peptides that are not conjugated to therapeutic agents, e.g., chemotherapeutic agents, hormones, vitamins, enzymes, toxins, steroids, apoptosis inducing agents, alkylating agents, antimetabolites, natural products and their derivatives, antisense oligonucleotide, neuroeffector (e.g., neurotransmitters or neurotransmitter antagonists), opiods, anti-inflammatory agents, diuretic, vasopressin agonist or antagonist, angiotensin, rennin, anti-bacterial agents, anti-viral agents, anti-coagulant, anti-thrombolytic agent, and anti-platelet agents.

Techniques for conjugating therapeutic moieties to peptides are well known. Moieties can be conjugated to peptides by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv. Drug Deliv. Rev. 53:171-216). Additional techniques for conjugating therapeutic moieties to peptides are well known, see, e.g., Amon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy,” in Monoclonal Antibodies For Cancer Detection And Therapy, Baldwin et al. (eds.), pp. 303-16 (Academic Press 1985), and Thorpe et al., 1982, Immunol. Rev. 62:119-58. Methods for fusing or conjugating antibodies to polypeptide moieties are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J. Immunol. 154:5590-5600; and Vil et al., 1992, PNAS 89:11337-11341. The fusion of a peptide to a therapeutic moiety does not necessarily need to be direct, but may occur through linker sequences. Such linker molecules are commonly known in the art and described in Section 5.1.3.2, supra, and in Denardo et al., 1998, Clin Cancer Res. 4:2483-90; Peterson et al., 1999, Bioconjug. Chem. 10:553; Zimmerman et al., 1999, Nucl. Med. Biol. 26:943-50; Garnett, 2002, Adv. Drug Deliv. Rev. 53:171-216, each of which is incorporated herein by reference in its entirety.

5.1.3 High Throughput Screening

High throughput screening can be used to screen chemical libraries for molecules, including, for example, EphB receptor binding peptides and EphB receptor binding compounds, that disrupt and/or prevent the formation of the EphB-EphrinB complex. One type of assay for identifying such molecules uses immobilized Eph receptor ectodomains in complex with ephrin-alkaline phosphatase fusion proteins. The ability to decrease bound alkaline phosphatase activity will identify molecule (e.g., an EphB receptor binding peptide or an EphB receptor binding compound) inhibitors of the EphB-Ephrin interaction. Standard high throughput screening methods are known to those skilled in the art and are also described in, e.g., U.S. Patent Publication No. US2006/0177452, which is incorporated by reference herein in its entirety. Further assays to assess binding affinity and biological activity of such identified molecules can be performed by standard methods known in the art and include those described in Section 5.3, infra.

5.2 Prophylactic/Therapeutic Methods

The Eph receptor binding peptides and/or EphB receptor binding compounds described herein (see Section 5.1, supra) can be administered to animals, including humans, to modulate EphB receptors in vivo. For example, certain peptides disclosed herein can be used to selectively activate or inhibit a member of the EphB receptor family, including but not limited to, EphB1, EphB2, EphB3, EphB4, EphB5 and EphB6. Thus, the present invention encompasses methods for therapeutic and prophylactic treatment of EphB receptor related diseases that comprise administering EphB receptor binding peptides and/or EphB receptor binding compounds that are agonists or antagonists in amounts sufficient to activate or inhibit an EphB receptor in vivo. Accordingly, the present invention provides methods for using the EphB receptor binding peptides and/or EphB receptor binding compounds to treat and/or manage EphB receptor related diseases, including, but not limited to, cancers, neoplastic diseases, vascular diseases (e.g., macular degeneration) and neurological disorders.

In the context of cancer, for example, the invention provides EphB receptor binding peptides and EphB receptor binding compounds that selectively bind to a member of the EphB receptor family, particularly EphB1 or EphB4, which act as agonists. EphB receptor agonists may bind to an EphB receptor, stimulate receptor clustering, transphosphorylation, downstream signaling, EphB receptor internalization and/or receptor degradation to inhibit tumor progression by inhibiting cell proliferation and metastasis. Moreover, the EphB receptor binding peptides and Eph receptor binding compounds described herein can be used to deliver cytotoxic agents to cancer tissues or cells, particularly where an EphB receptor is overexpressed in the cancer cells compared to normal, non-cancerous cells of the same tissue type or cells from the same patient. The EphB receptor binding peptides can therefore be coupled to chemotherapeutic drugs, toxins, or pro-apoptotic peptides to decrease tumor growth, suppress clinical arthritis, or destroy prostate tissue (Arap, W. et al. 1998 Science 279:377-380; Olson, T. A. et al 1997 Int J Cancer 73:865-870; Ellerby, H. M. et al. 1999 Nat Med 5:1032-1038; Arap, W. et al. 2002 PNAS USA 99:1527-1531; Gerlag, D. M. et al. 2001 Arthritis Research 3:357-361). Toxic or pro-apoptotic substances can also be delivered intracellularly to selectively kill cells (Ellerby, H. M. et al. 1999 Nat Med 5:1032-1038). In specific embodiments, the invention provides EphB receptor binding peptides which are conjugated, linked, and/or fused to a therapeutic moiety which can be used to kill/eliminate/modulate cells expressing an EphB receptor to prevent, treat and/or manage an EphB related disease characterized by aberrant proliferation of such cells.

In a specific embodiment, an EphB receptor binding peptide or EphB receptor binding compound described herein, and in particular Section 5.1, is an antagonist. In a specific embodiment, an antagonist is an EphB receptor binding peptide or EphB receptor binding compound that competes with and/or inhibits binding of a natural ligand (e.g., an EphrinB ligand) to a member of the EphB family of receptors and prevents, inhibits or reduces EphB receptor clustering and/or transphosphorylation and activation of downstream signaling pathways. EphB receptor antagonists may function by preventing Ephrin-dependent Eph receptor clustering and transphosphorylation, which are necessary steps to activate downstream signaling pathways (Murai & Pasquale, 2003, J. Cell Sci. 116:2823-2832). In a specific embodiment, an antagonist is an EphB receptor binding compound that is an Fc fusion protein, as described in Section 5.1, supra. In another embodiment, an antagonist is an EphB receptor binding compound that is a fragment of a peptide. In another embodiment, an antagonist is an EphB receptor binding compound that is pegylated.

Accordingly, in certain embodiments, the EphB receptor binding peptides and EphB binding compounds can be used to prevent, treat and/or manage diseases which involve aberrant angiogenesis, such as cancer and non-neoplastic conditions such as, for example, cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis.

In one embodiment, the EphB receptor binding peptides and/or Eph receptor binding compound can be administered in combination with one or more other therapies useful in the treatment and/or management of cancer. In certain embodiments, one or more EphB receptor binding peptides or Eph receptor binding compounds are administered to an animal, e.g., a mammal, preferably a human, concurrently with one or more other therapies useful for the treatment of cancer. In other embodiments, one or more EphB receptor binding peptides and/or Eph receptor binding compounds are administered to an animal, e.g., a mammal, preferably a human, concurrently with one or more other therapies useful for the treatment of a vascular disease (e.g., macular degeneration). In yet other embodiments, one or more EphB receptor binding peptides or Eph receptor binding compounds are administered to an animal, e.g., a mammal, preferably a human, concurrently with one or more other therapies (useful for the treatment of a neurological disorder. The term “concurrently” is not limited to the administration of therapies, e.g., prophylactic or therapeutic agents at exactly the same time, but rather it is meant that the EphB receptor binding peptide or Eph receptor binding compound and the other agent are administered to a subject in a sequence and within a time interval such that the antibodies can act together with the other agent to provide an increased benefit than if they were administered otherwise. For example, each therapy, e.g., prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route. In other embodiments, the EphB receptor binding peptides and/or Eph receptor binding compounds are administered before, concurrently or after surgery. In particular embodiments, the surgery completely removes localized tumors or reduces the size of large tumors. Surgery can also be done as a preventive measure or to relieve pain. In certain embodiments, one or more EphB receptor binding peptides or Eph receptor binding compounds are administered to an animal, e.g., a mammal, preferably a human, after removal of a tumor by surgery. In other embodiments, one or more EphB receptor binding peptides or Eph receptor binding compounds are administered to an animal, e.g., a mammal, preferably a human, after a tumor is treated locally with radiation therapy and/or chemotherapy. In some embodiments, one or more EphB receptor binding peptides or Eph receptor binding compounds are administered to an animal, e.g., a mammal, preferably a human, following removal of a tumor by surgery and in combination with adjuvant therapy.

In various embodiments, the therapies, e.g., prophylactic or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In specific embodiments, two or more components are administered within the same patient visit.

The dosage amounts and frequencies of administration provided herein are encompassed by the terms therapeutically effective and prophylactically effective. The dosage and frequency further will typically vary according to factors specific for each patient depending on the specific therapies, e.g., therapeutic or prophylactic agents administered, the severity and type of cancer, the route of administration, as well as age, body weight, response, and the past medical history of the patient. Suitable regimens can be selected by one skilled in the art by considering such factors and by following, for example, dosages reported in the literature and recommended in the Physicians' Desk Reference (61^{st} ed., 2007).

5.2.1 Patient Population

The present invention encompasses methods for preventing, treating and/or managing an EphB receptor related disease, or a symptom thereof, in a subject, which method comprises administering one or more EphB receptor binding peptides and/or EphB receptor binding compounds, particularly those described in Section 5.1, supra. The subject is preferably a mammal such as non-primate (e.g., cows, pigs, horses, cats, dogs, rats, etc.) or a primate (e.g., monkeys, such as cynomolgous monkeys, or humans). In a preferred embodiment, the subject is a human.

The methods comprise the administration of one or more EphB receptor binding peptides and/or EphB receptor binding compounds to patients suffering from or expected to suffer from (e.g., patients with a genetic predisposition for or patients that have previously suffered from) an EphB receptor related disease. In a certain embodiment, the subject is diagnosed with an EphB receptor related disease. Non-limiting examples of EphB receptor related diseases include cancer, and vascular diseases (e.g., macular degeneration), and neurological disorders. Such patients may have been previously treated or are currently being treated for the EphB receptor related disease, e.g., with a non-EphB receptor binding therapy. In accordance with the invention, an EphB receptor binding peptide and/or EphB receptor binding compound may be used as any line of therapy, including, but not limited to, a first, second, third and fourth line of therapy. Further, in accordance with the invention, an EphB receptor binding peptide and/or EphB receptor binding compound can be used before any adverse effects or intolerance of the non-EphB receptor binding therapies occur. The invention encompasses methods for administering one or more EphB receptor binding peptides and/or EphB receptor binding compounds to prevent the onset or recurrence of an EphB receptor related disease (e.g., cancer).

In one embodiment, the invention also provides methods of treatment or management of an EphB receptor related disease as alternatives to current therapies (e.g., therapeutic or prophylactic agents). In a specific embodiment, the current therapy has proven or may prove too toxic (i.e., results in unacceptable or unbearable side effects) for the patient. In another embodiment, an EphB receptor binding peptide and/or EphB receptor binding compound decreases the side effects as compared to the current therapy. In another embodiment, the patient has proven refractory or non-responsive to a current therapy. In certain embodiments, one or more EphB receptor binding peptides and/or EphB receptor binding compounds can be administered to a patient in need thereof instead of another therapy to treat an EphB receptor related disease.

The present invention also encompasses methods for administering one or more EphB receptor binding peptides and/or EphB receptor binding compounds to treat or ameliorate an EphB receptor related disease or symptoms thereof in patients that are or have become refractory to non-EphB receptor binding peptide and/or non-EphB receptor binding compound therapies. The determination of whether the EphB receptor related disease or symptoms thereof are refractory can be made either in vivo or in vitro by any method known in the art for assaying the effectiveness of a therapy on affected cells in the EphB receptor related disease, e.g., epithelial and/or endothelial cells, or in patients that are or have become refractory to non-EphB receptor binding peptide and/or non-EphB receptor binding compound therapies.

In various embodiments, a cancer is refractory where the number of cancer cells has not been significantly reduced, or has increased. The invention also encompasses methods for administering one or more EphB receptor binding peptides and/or EphB receptor binding compounds, particularly, those described in Section 5.1, supra, to prevent the recurrence, onset or development of cancer in patients predisposed to having cancer.

In particular embodiments, the EphB receptor binding peptides and/or EphB receptor binding compounds, or other therapeutics that reduce EphB expression and/or activity, are administered to reverse resistance or reduced sensitivity of cancer cells to certain hormonal, radiation and chemotherapeutic agents thereby resensitizing the cancer cells to one or more of these agents, which can then be administered (or continue to be administered) to treat or manage cancer, including to prevent metastasis. In other embodiments, provided herein are methods for treating metastasis in a patient in need thereof comprising administering one or more EphB receptor binding peptides and/or EphB receptor binding compounds, particularly, those described in Section 5.1.

In alternate embodiments, the invention provides methods for treating patients' cancer by administering one or more EphB receptor binding peptides and/or EphB receptor binding compounds in combination with any other treatment or to patients who have proven refractory to other treatments but are no longer on these treatments. In certain embodiments, the patients being treated by the methods described herein are patients already being treated with chemotherapy, radiation therapy, hormonal therapy, or biological therapy/immunotherapy. Among these patients are refractory patients and those with cancer despite treatment with existing cancer therapies.

In specific embodiments, the existing treatment is chemotherapy. In particular embodiments, the existing treatment includes administration of chemotherapies including, but not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, docetaxel, etc. Among these patients are patients treated with radiation therapy, hormonal therapy and/or biological therapy/immunotherapy. Also among these patients are those who have undergone surgery for the treatment of cancer.

Alternatively, the invention also encompasses methods for treating patients undergoing or having undergone radiation therapy. Among these are patients being treated or previously treated with chemotherapy, hormonal therapy and/or biological therapy/immunotherapy. Also among these patients are those who have undergone surgery for the treatment of cancer.

In other embodiments, the invention encompasses methods for treating patients undergoing or having undergone hormonal therapy and/or biological therapy/immunotherapy. Among these are patients being treated or having been treated with chemotherapy and/or radiation therapy. Also among these patients are those who have undergone surgery for the treatment of cancer.

Additionally, the invention also provides methods of treatment of cancer as an alternative to chemotherapy, radiation therapy, hormonal therapy, and/or biological therapy/immunotherapy where the therapy has proven or may prove too toxic, i.e., results in unacceptable or unbearable side effects, for the subject being treated. The subject being treated with the methods described herein may, optionally, be treated with other cancer treatments such as surgery, chemotherapy, radiation therapy, hormonal therapy or biological therapy, depending on which treatment was found to be unacceptable or unbearable.

In other embodiments, the invention provides administration of one or more Eph receptor binding peptides and/or Eph receptor binding compounds without any other cancer therapies for the treatment of cancer, but who have proved refractory to such treatments. In specific embodiments, patients refractory to other cancer therapies are administered one or more Eph receptor binding compounds in the absence of cancer therapies.

5.2.2 EphB Receptor Related Diseases

The present invention encompasses methods for treating and/or managing an EphB receptor related disease in a subject comprising administering one or more Eph receptor binding peptides and/or Eph receptor binding compounds either alone or in combination with one or more other therapies (e.g., therapeutic or prophylactic agents). Non-limiting examples of EphB receptor related diseases include cancer, and vascular diseases (e.g., macular degeneration) and neurological disorders (e.g., spinal cord injury). Non-limiting diseases involving aberrant angiogenesis are also contemplated by the methods of the present invention, and include cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. In one embodiment, an EphB receptor related disease is an EphB4 receptor related disease.

5.2.2.1 Cancers

Cancers and related disorders that can be treated, prevented, managed and/or ameliorated by methods and compositions of the present invention include, but are not limited to, cancers of an epithelial or endothelial cell origin. Non-limiting examples of such cancers include mesothelioma, ovarian cancer, bladder cancer, squamous cell carcinoma of the head and neck, breast cancer, prostate cancer, colon cancer, small cell lung carcinoma and cancers of neurological origin. In a specific embodiment, the cancers to be treated and/or managed overexpress one or more members of the EphB receptor family.

Other non-limiting examples of such cancers include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions The Complete Book of Cancer Diagnosis, Treatment, and Recovery, Viking Penguin, Penguin Books U.S.A., Inc., United States of America).

Accordingly, the methods and compositions are also useful in the treatment, prevention, and/or management of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. It is also contemplated that cancers caused by aberrations in apoptosis would also be treated by the methods and compositions described herein. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the skin, lung, colon, breast, prostate, bladder, kidney, pancreas, ovary, or uterus. In other specific embodiments, sarcoma, melanoma, or leukemia is treated, prevented, and/or managed.

In some embodiments, the cancer is characterized by or displays aberrant EphB receptor expression. In specific embodiments, the cancer is malignant and overexpresses an EphB receptor, preferably EphB1 or EphB4. In one embodiment, aberrant expression of an EphB receptor in a sample (e.g., cancer cells or cancerous tissues) relative to a negative control (e.g., non-cancerous cells, normal tissue samples) is detected via assays well known in the art and/or described herein. Non-limiting examples of such assays include immunohistochemistry, ELISA, immunoflurescence, Western blots, and flow cytometry. In a specific embodiment, the EphB receptor expression level of a cancer that overexpresses an EphB receptor is at least 10%, at least 20%, at least 30%, at least 40%, at least 50%, at least 60%, at least 70%, at least 80%, at least 90%, or at least 100% higher than an EphB receptor expression level of a negative control (e.g., normal or non-cancerous cells or normal or non-cancerous tissue samples) as determined using assays well known in the art and/or described herein.

5.2.3 Other Prophylactic/Therapeutic Agents

In some embodiments, the administration of one or more EphB receptor binding peptides and/or EphB receptor binding compounds is combined with the administration of one or more therapies such as, but not limited to, chemotherapies, radiation therapies, hormonal therapies, and/or biological therapies/immunotherapies. Prophylactic/therapeutic agents include, but are not limited to, proteinaceous molecules, including, but not limited to, peptides, polypeptides, proteins, including post-translationally modified proteins, antibodies, intrabodies, aptamers, etc.; or small molecules (less than 1000 daltons), inorganic or organic compounds; or nucleic acid molecules including, but not limited to, double-stranded or single-stranded DNA, or double-stranded or single-stranded RNA, RNAi, as well as triple helix nucleic acid molecules. Prophylactic/therapeutic agents can be derived from any known organism (including, but not limited to, animals, plants, bacteria, fungi, and protista, or viruses) or from a library of synthetic molecules. For non-limiting examples of other prophylactic and therapeutic agents for use in the methods of the present invention, see U.S. Patent Publication Nos. US 2004/0091486 A1 and US 2004/0028685 A1, and US 2006/0121042 A1, which is each incorporated by reference herein in its entirety.

In a specific embodiment, the methods described herein encompass administration of an EphB receptor binding peptide and/or an EphB receptor binding compound in combination with the administration of one or more prophylactic/therapeutic agents that are inhibitors of kinases such as, but not limited to, ABL, ACK, AFK, AKT (e.g., AKT-1, AKT-2, and AKT-3), ALK, AMP-PK, ATM, Aurora1, Aurora2, bARK1, bArk2, BLK, BMX, BTK, CAK, CaM kinase, CDC2, CDK, CK, COT, CTD, DNA-PK, EGF-R, ErbB-1, ErbB-2, ErbB-3, ErbB-4, ERK (e.g., ERK1, ERK2, ERK3, ERK4, ERK5, ERK6, ERK7), ERT-PK, FAK, FGR (e.g., FGF1R, FGF2R), FLT (e.g., FLT-1, FLT-2, FLT-3, FLT-4), FRK, FYN, GSK (e.g., GSK1, GSK2, GSK3-alpha, GSK3-beta, GSK4, GSK5), G-protein coupled receptor kinases (GRKs), HCK, HER2, HKII, JAK (e.g., JAK1, JAK2, JAK3, JAK4), JNK (e.g., JNK1, JNK2, JNK3), KDR, KIT, IGF-1 receptor, IKK-1, IKK-2, INSR (insulin receptor), IRAK1, IRAK2, IRK, ITK, LCK, LOK, LYN, MAPK, MAPKAPK-1, MAPKAPK-2, MEK, MET, MFPK, MHCK, MLCK, MLK3, NEU, NIK, PDGF receptor alpha, PDGF receptor beta, PHK, PI-3 kinase, PKA, PKB, PKC, PKG, PRK1, PYK2, p38 kinases, p135tyk2, p34cdc2, p42cdc2, p42mapk, p44 mpk, RAF, RET, RIP, RIP-2, RK, RON, RS kinase, SRC, SYK, S6K, TAK1, TEC, TIE1, TIE2, TRKA, TXK, TYK2, UL13, VEGFR1, VEGFR2, YES, YRK, ZAP-70, and all subtypes of these kinases (see e.g., Hardie and Hanks (1995) The Protein Kinase Facts Book, I and II, Academic Press, San Diego, Calif.). In specific embodiments, an EphB receptor binding peptide and/or and/or EphB receptor binding compound is administered in combination with the administration of one or more prophylactic/therapeutic agents that are inhibitors of Eph receptor kinases (e.g., EphB1, EphB2, EphB3, EphB4, EphB5 and EphB6). In a specific embodiment, an EphB receptor binding peptide and/or EphB receptor binding compound is administered in combination with the administration of one or more prophylactic/therapeutic agents that are inhibitors of an EphB receptor, preferably EphB1, EphB2, and EphB4.

In another specific embodiment, the invention provides for methods that encompass administration of an EphB receptor binding peptide and/or EphB receptor binding compound in combination with the administration of one or more prophylactic/therapeutic agents that are angiogenesis inhibitors such as, but not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16k_{d} fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-β); Vasculostatin; Vasostatin (calreticulin fragment); ZD6126; ZD6474; farnesyl transferase inhibitors (FTI); and bisphosphonates.

In another specific embodiment, the invention provides for methods that encompass administration of an EphB receptor binding peptide and/or EphB receptor binding compound in combination with the administration of one or more prophylactic/therapeutic agents that are anti-cancer agents such as, but not limited to: acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decarbazine, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, fluorocitabine, fosquidone, fostriecin sodium, gemcitabine, gemcitabine hydrochloride, hydroxyurea, idarubicin hydrochloride, ifosfamide, ilmofosine, interleukin 2 (including recombinant interleukin 2, or rIL2), interferon alpha-2a, interferon alpha-2b, interferon alpha-n1, interferon alpha-n3, interferon beta-I a, interferon gamma-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nitrosoureas, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride. Other anti-cancer drugs include, but are not limited to: 20-epi-1,25 dihydroxyvitamin D3,5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1, antiandrogens, antiestrogens, antineoplaston, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chloroquinoxaline sulfonamide, cicaprost, cis-porphyrin, cladribine, clomifene analogs, clotrimazole, collismycin A, collismycin B, combretastatin A4, combretastatin analog, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexamethasone, dexifosfamide, dexrazoxane, dexverapamil, diaziquone, didemnin B, didox, diethylnorspermine, dihydro-5-azacytidine, dihydrotaxol, dioxamycin, diphenyl spiromustine, docetaxel, docosanol, dolasetron, doxifluridine, droloxifene, dronabinol, duocarmycin SA, ebselen, ecomustine, edelfosine, edrecolomab, eflornithine, elemene, emitefur, epirubicin, epristeride, estramustine analog, estrogen agonists, estrogen antagonists, etanidazole, etoposide phosphate, exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analog, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogs, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance gene inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, O6-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, prednisone, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sd±1 mimetics, semustine, senescence derived inhibitor 1, sense oligonucleotides, signal transduction inhibitors, signal transduction modulators, single chain antigen binding protein, sizofuran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfinosine, superactive vasoactive intestinal peptide antagonist, suradista, suramin, swainsonine, synthetic glycosaminoglycans, tallimustine, tamoxifen methiodide, tauromustine, taxol, tazarotene, tecogalan sodium, tegafur, tellurapyrylium, telomerase inhibitors, temoporfin, temozolomide, teniposide, tetrachlorodecaoxide, tetrazomine, thaliblastine, thalidomide, thiocoraline, thioguanine, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, and zinostatin stimalamer. In a specific embodiment, additional anti-cancer drugs are 5-fluorouracil and leucovorin.

In more particular embodiments, the present invention also comprises the administration of one or more EphB receptor binding peptides and/or EphB receptor binding compounds in combination with the administration of one or more therapies such as, but not limited to anti-cancer agents such as those disclosed in Table 2 or those discussed below in Section 5.2.3.1.

TABLE 2

| Therapeutic Agent | Administration | Dose | Intervals |
|---|---|---|---|
| doxorubicin | Intravenous | 60-75 mg/m² on Day 1 | 21 day intervals |
| hydrochloride | | | |
| (Adriamycin | | | |
| RDF ® and | | | |
| Adriamycin | | | |
| PFS ®) | | | |
| epirubicin | Intravenous | 100-120 mg/m² on Day | 3-4 week cycles |
| hydrochloride | | 1 of each cycle or | |
| (Ellence ™) | | divided equally and | |
| | | given on Days 1-8 of the | |
| | | cycle | |
| fluorousacil | Intravenous | How supplied: | |
| | | 5 ml and 10 ml vials | |
| | | (containing 250 and 500 mg | |
| | | flourouracil | |
| | | respectively) | |
| docetaxel | Intravenous | 60-100 mg/m² over 1 | Once every 3 weeks |
| (Taxotere ®) | | hour | |
| paclitaxel | Intravenous | 175 mg/m² over 3 hours | Every 3 weeks for 4 |
| (Taxol ®) | | | courses (administered |
| | | | sequentially to |
| | | | doxorubicin-containing |
| | | | combination |
| | | | chemotherapy) |
| tamoxifen citrate | Oral | 20-40 mg | Daily |
| (Nolvadex ®) | (tablet) | Dosages greater than 20 mg | |
| | | should be given in | |
| | | divided doses (morning | |
| | | and evening) | |
| leucovorin | Intravenous or | How supplied: | Dosage is unclear from |
| calcium for | intramuscular | 350 mg vial | text. PDR 3610 |
| injection | injection | | |
| luprolide acetate | Single | 1 mg (0.2 ml or 20 unit | Once a day |
| (Lupron ®) | subcutaneous | mark) | |
| | injection | | |
| flutamide | Oral (capsule) | 250 mg | 3 times a day at 8 hour |
| (Eulexin ®) | | (capsules contain 125 mg | intervals (total daily |
| | | flutamide each) | dosage 750 mg) |
| nilutamide | Oral | 300 mg or 150 mg | 300 mg once a day for |
| (Nilandron ®) | (tablet) | (tablets contain 50 or | 30 days followed by |
| | | 150 mg nilutamide each) | 150 mg once a day |
| bicalutamide | Oral | 50 mg | Once a day |
| (Casodex ®) | (tablet) | (tablets contain 50 mg | |
| | | bicalutamide each) | |
| progesterone | Injection | USP in sesame oil 50 mg/ml | |
| ketoconazole | Cream | 2% cream applied once | |
| (Nizoral ®) | | or twice daily depending | |
| | | on symptoms | |
| prednisone | Oral | Initial dosage may vary | |
| | (tablet) | from 5 mg to 60 mg per | |
| | | day depending on the | |
| | | specific disease entity | |
| | | being treated. | |
| estramustine | Oral | 14 mg/kg of body | Daily given in 3 or 4 |
| phosphate sodium | (capsule) | weight (i.e. one 140 mg | divided doses |
| (Emcyt ®) | | capsule for each 10 kg | |
| | | or 22 lb of body weight) | |
| etoposide or VP- | Intravenous | 5 ml of 20 mg/ml | |
| 16 | | solution (100 mg) | |
| dacarbazine | Intravenous | 2-4.5 mg/kg | Once a day for 10 days. |
| (DTIC-Dome ®) | | | May be repeated at 4 |
| | | | week intervals |
| polifeprosan 20 | wafer placed in | 8 wafers, each | |
| with carmustine | resection | containing 7.7 mg of | |
| implant (BCNU) | cavity | carmustine, for a total of | |
| (nitrosourea) | | 61.6 mg, if size and | |
| (Gliadel ®) | | shape of resection cavity | |
| | | allows | |
| cisplatin | Injection | [n/a in PDR 861] | |
| | | How supplied: | |
| | | solution of 1 mg/ml in | |
| | | multi-dose vials of | |
| | | 50 mL and 100 mL | |
| mitomycin | Injection | supplied in 5 mg and 20 mg | |
| | | vials (containing 5 mg | |
| | | and 20 mg | |
| | | mitomycin) | |
| gemcitabine HCl | Intravenous | For NSCLC-2 | 4 week schedule- |
| (Gemzar ®) | | schedules have been | Days 1, 8 and 15 of each |
| | | investigated and the | 28-day cycle. Cisplatin |
| | | optimum schedule has | intravenously at 100 mg/m² |
| | | not been determined | on day 1 after |
| | | 4 week schedule- | the infusion of Gemzar. |
| | | administration | 3 week schedule- |
| | | intravenously at 1000 mg/m² | Days 1 and 8 of each 21 |
| | | over 30 minutes | day cycle. Cisplatin at |
| | | on 3 week schedule- | dosage of 100 mg/m² |
| | | Gemzar administered | administered |
| | | intravenously at 1250 mg/m² | intravenously after |
| | | over 30 minutes | administration of |
| | | | Gemzar on day 1. |
| carboplatin | Intravenous | Single agent therapy: | Every 4 weeks |
| (Paraplatin ®) | | 360 mg/m² I.V. on day 1 | |
| | | (infusion lasting 15 | |
| | | minutes or longer) | |
| | | Other dosage | |
| | | calculations: | |
| | | Combination therapy | |
| | | with cyclophosphamide, | |
| | | Dose adjustment | |
| | | recommendations, | |
| | | Formula dosing, etc. | |
| ifosamide | Intravenous | 1.2 g/m² daily | 5 consecutive days |
| (Ifex ®) | | | Repeat every 3 weeks |
| | | | or after recovery from |
| | | | hematologic toxicity |
| topotecan | Intravenous | 1.5 mg/m² by | 5 consecutive days, |
| hydrochloride | | intravenous infusion | starting on day 1 of 21 |
| (Hycamtin ®) | | over 30 minutes daily | day course |

The invention also encompasses administration of the EphB receptor binding peptides and/or EphB receptor binding compounds in combination with radiation therapy comprising the use of x-rays, gamma rays and other sources of radiation to destroy the cancer cells. In specific embodiments, the radiation treatment is administered as external beam radiation or teletherapy wherein the radiation is directed from a remote source. In other specific embodiments, the radiation treatment is administered as internal therapy or brachytherapy wherein a radioactive source is placed inside the body close to cancer cells or a tumor mass.

Cancer therapies and their dosages, routes of administration and recommended usage are known in the art and have been described in such literature as the Physicians' Desk Reference (61^{st} ed., 2007) and in Section 5.4, infra.

5.2.3.1 Anti-Cancer Agents

Any therapy (e.g., therapeutic or prophylactic agent) which is known to be useful, has been used, or is currently being used for the prevention, treatment, and/or management of a proliferative disorder, such as cancer (benign, malignant or metastatic), or one or more symptoms thereof can be used in compositions and methods described herein. Therapies (e.g., therapeutic or prophylactic agents) include, but are not limited to, peptides, polypeptides, fusion proteins, nucleic acid molecules, small molecules, mimetic agents, synthetic drugs, inorganic molecules, and organic molecules. Non-limiting examples of cancer therapies include chemotherapies, radiation therapies, hormonal therapies, and/or biological therapies/immunotherapies.

In certain embodiments, the anti-cancer agent is an immunomodulatory agent, such as a chemotherapeutic agent. In certain other embodiments, the anti-cancer agent is an immunomodulatory agent other than a chemotherapeutic agent. In other embodiments, the anti-cancer agent is not an immunomodulatory agent. In specific embodiments, the anti-cancer agent is an anti-angiogenic agent. In other embodiments, the anti-cancer agent is not an anti-angiogenic agent. In specific embodiments, the anti-cancer agent is an anti-inflammatory agent. In other embodiments, the anti-cancer agent is not an anti-inflammatory agent.

In particular embodiments, the anti-cancer agent is, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; EphA2 inhibitors (e.g., anti-EphA2 antibodies that result in the phosphorylation of EphA2 and the degradation of EphA2 (see, U.S. Patent Publication Nos. US 2004/0091486 A1 and US 2004/0028685 A1, and US 2006/0121042 A1, which is each incorporated by reference herein in its entirety); elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriec in sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; anti-CD2 antibodies (e.g., siplizumab (MedImmune Inc.; International Publication No. WO 02/098370, which is incorporated herein by reference in its entirety)); megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogs; clotrimazole; collismycin A; collismycin B; combretastatin A4; combretastatin analog; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analog; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP (Biogen, Cambridge, Mass.; International Publication No. WO 93/0686 and U.S. Pat. No. 6,162,432); liarozole; linear polyamine analog; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogs; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogs; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sd±1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; VITAXIN™ (see U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents”); vorozole; zanoterone; zeniplatin; zilascorb; and zinostatin stimalamer.

Anticancer agents described herein can be cytotoxic agents or cancer chemotherapeutic agents. As non limiting examples, cytotoxic agents that target a DNA associated process encompass cyclophosphamide, melphalan, mitomycin C, bizelesin, cisplatin, doxorubicin, etoposide, mitoxantrone, SN 38, Et 743, actinomycin D, bleomycin and TLK286. Cancer chemotherapeutic agents can be, without limitation, a taxane such as docetaxel; an anthracyclin such as doxorubicin; an alkylating agent; a vinca alkaloid; an anti metabolite; a platinum agent such as cisplatin or carboplatin; a steroid such as methotrexate; an antibiotic such as adriamycin; a isofamide; or a selective estrogen receptor modulator; an antibody such as trastuzumab.

Taxanes are chemotherapeutic agents useful in the combination treatment. Useful taxanes include, without limitation, docetaxel (Taxotere; Aventis Pharmaceuticals, Inc.; Parsippany, N.J.) and paclitaxel (Taxol; Bristol Myers Squibb; Princeton, N.J.). See, for example, Chan et al. 1999 J Clin Oncol 17:2341 2354, and Paridaens et al. 2000 J Clin Oncol 18:724.

Another cancer chemotherapeutic agent useful in the combination treatment is an anthracyclin such as doxorubicin, idarubicin or daunorubicin. Doxorubicin is a commonly used cancer chemotherapeutic agent and can be useful, for example, for treating breast cancer (Stewart and Ratain, In: “Cancer: Principles and Practice of Oncology” 5th ed., chap. 19, eds. DeVita, Jr. et al.; J.P. Lippincott 1997; Harris et al., In: “Cancer: Principles and practice of oncology,” supra, 1997). In addition, doxorubicin has anti angiogenic activity (Folkman, 1997 Nature Biotechnology 15:510; Steiner, In: “Angiogenesis: Key principles Science, technology and medicine,” pp. 449-454, eds. Steiner et al., Birkhauser Verlag, 1992), which can contribute to its effectiveness in treating cancer.

Alkylating agents such as melphalan or chlorambucil are cancer chemotherapeutic agents useful in the combination treatment. Similarly, a vinca alkaloid such as vindesine, vinblastine or vinorelbine; or an antimetabolite such as 5 fluorouracil, 5 fluorouridine or a derivative thereof are cancer chemotherapeutic agents useful in the combination treatment.

Platinum agents are chemotherapeutic agents useful in the combination treatment. Such a platinum agent can be, for example, cisplatin or carboplatin as described, for example, in Crown, 2001 Seminars in Oncol 28:28-37. Other cancer chemotherapeutic agents useful in the combination treatment include, without limitation, methotrexate, mitomycin C, adriamycin, ifosfamide and ansamycins.

Cancer chemotherapeutic agents used for treatment of breast cancer and other hormonally dependent cancers also can be used as an agent that antagonizes the effect of estrogen, such as a selective estrogen receptor modulator or an anti estrogen. The selective estrogen receptor modulator, tamoxifen, is a cancer chemotherapeutic agent that can be used in the combination treatment for treatment of breast cancer (Fisher et al. 1998 J Natl Cancer Instit 90:1371 1388).

A therapeutic agent useful in the combination treatment can be an antibody such as a humanized monoclonal antibody. As an example, the anti epidermal growth factor receptor 2 (HER2) antibody, trastuzumab (Herceptin; Genentech, South San Francisco, Calif.) is a therapeutic agent useful in a conjugate for treating HER2/neu overexpressing breast cancers (White et al. 2001 Ann Rev Med 52:125-141).

Another therapeutic agent useful in the invention also can be a cytotoxic agent, which, as used herein, is any molecule that directly or indirectly promotes cell death. Cytotoxic agents useful in the invention include, without limitation, small molecules, polypeptides, peptides, peptidomimetics, nucleic acid molecules, cells and viruses. As non limiting examples, cytotoxic agents useful in the invention include cytotoxic small molecules such as doxorubicin, docetaxel or trastuzumab; antimicrobial peptides such as those described further below; pro-apoptotic polypeptides such as caspases and toxins, for example, caspase 8; diphtheria toxin A chain, Pseudomonas exotoxin A, cholera toxin, ligand fusion toxins such as DAB389EGF, ricinus communis toxin (ricin); and cytotoxic cells such as cytotoxic T cells. See, for example, Martin et al. 2000 Cancer Res 60:3218-3224; Kreitman and Pastan 1997 Blood 90:252-259; Allam et al. 1997 Cancer Res 57:2615-2618; Osborne and Coronado Heinsohn 1996 Cancer J Sci Am 2:175. One skilled in the art understands that these and additional cytotoxic agents described herein or known in the art can be useful as therapeutic agents.

In specific embodiments, radiation therapy comprising the use of x-rays, gamma rays and other sources of radiation to destroy the cancer cells is used in combination with the EphB receptor binding peptides and/or EphB receptor binding compounds. In specific embodiments, the radiation treatment is administered as external beam radiation or teletherapy, wherein the radiation is directed from a remote source. In other specific embodiments, the radiation treatment is administered as internal therapy or brachytherapy wherein a radioactive source is placed inside the body close to cancer cells or a tumor mass.

Cancer therapies and their dosages, routes of administration and recommended usage are known in the art and have been described in such literature as the Physicians' Desk Reference (61^{st} ed., 2007).

5.2.3.2 Anti-Angiogenesis Agents

In certain embodiments, the present invention provides compositions comprising one or more EphB receptor binding peptides and/or EphB receptor binding compounds and one or more anti-angiogenic agents, and methods for treating and/or managing an EphB related disease (e.g., aberrant angiogenesis) in a subject comprising the administration of said compositions. Any anti-angiogenic agent well-known to one of skill in the art can be used in the compositions and methods described herein.

Any anti-angiogenic agent well-known to one of skill in the art can be used in the compositions and methods described herein. Non-limiting examples of anti-angiogenic agents include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to TNF-α, nucleic acid molecules (e.g., antisense molecules or triple helices), organic molecules, inorganic molecules, and small molecules that reduce or inhibit angiogenesis. In particular, examples of anti-angiogenic agents, include, but are not limited to, endostatin, angiostatin, apomigren, anti-angiogenic antithrombin III, the 29 kDa N-terminal and a 40 kDa C-terminal proteolytic fragments of fibronectin, a uPA receptor antagonist, the 16 kDa proteolytic fragment of prolactin, the 7.8 kDa proteolytic fragment of platelet factor-4, the anti-angiogenic 24 amino acid fragment of platelet factor-4, the anti-angiogenic factor designated 13.40, the anti-angiogenic 22 amino acid peptide fragment of thrombospondin I, the anti-angiogenic 20 amino acid peptide fragment of SPARC, RGD and NGR containing peptides, the small anti-angiogenic peptides of laminin, fibronectin, procollagen and EGF, integrin αvβ3 antagonists, acid fibroblast growth factor (aFGF) antagonists, basic fibroblast growth factor (bFGF) antagonists, vascular endothelial growth factor (VEGF) antagonists (e.g., anti-VEGF antibodies (e.g., AVASTIN™ (Genentech)), VEGF receptor (VEGFR) antagonists (e.g., anti-VEGFR antibodies) and anti-integrin antagonists (e.g., REOPRO® (abciximab) (Centocor) which binds to the glycoprotein IIb/IIIa receptor on the platelets for the prevention of clot formation).

Non-limiting examples of anti-angiogenic agents include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to TNF-α, nucleic acid molecules (e.g., antisense molecules or triple helices), organic molecules, inorganic molecules, and small molecules that reduce or inhibit angiogenesis. In particular, examples of anti-angiogenic agents, include, but are not limited to, endostatin, angiostatin, apomigren, anti-angiogenic antithrombin III, the 29 kDa N-terminal and a 40 kDa C-terminal proteolytic fragments of fibronectin, a uPA receptor antagonist, the 16 kDa proteolytic fragment of prolactin, the 7.8 kDa proteolytic fragment of platelet factor-4, the anti-angiogenic 24 amino acid fragment of platelet factor-4, the anti-angiogenic factor designated 13.40, the anti-angiogenic 22 amino acid peptide fragment of thrombospondin I, the anti-angiogenic 20 amino acid peptide fragment of SPARC, RGD and NGR containing peptides, the small anti-angiogenic peptides of laminin, fibronectin, procollagen and EGF, integrin α_{V}β₃ antagonists, acid fibroblast growth factor (aFGF) antagonists, basic fibroblast growth factor (bFGF) antagonists, vascular endothelial growth factor (VEGF) antagonists (e.g., anti-VEGF antibodies), and VEGF receptor (VEGFR) antagonists (e.g., anti-VEGFR antibodies).

Examples of integrin α_{V}β₃ antagonists include, but are not limited to, proteinaceous agents such as non-catalytic metalloproteinase fragments, RGD peptides, peptide mimetics, fusion proteins, disintegrins or derivatives or analogs thereof, and antibodies that immunospecifically bind to integrin α_{V}β₃, nucleic acid molecules, organic molecules, and inorganic molecules. Non-limiting examples of antibodies that immunospecifically bind to integrin α_{V}β₃ include 11D2 (Searle), LM609 (Scripps), and VITAXIN™ (Medlmmune, Inc.). Non-limiting examples of small molecule peptidometric integrin αVβ₃ antagonists include S836 (Searle) and S448 (Searle). Examples of disintegrins include, but are not limited to, Accutin. The invention also encompasses the use of any of the integrin α_{V}β₃ antagonists disclosed in the following U.S. Patents and International publications in the compositions and methods described herein: U.S. Pat. Nos. 5,149,780; 5,196,511; 5,204,445; 5,262,520; 5,306,620; 5,478,725; 5,498,694; 5,523,209; 5,578,704; 5,589,570; 5,652,109; 5,652,110; 5,693,612; 5,705,481; 5,753,230; 5,767,071; 5,770,565; 5,780,426; 5,817,457; 5,830,678; 5,849,692; 5,955,572; 5,985,278; 6,048,861; 6,090,944; 6,096,707; 6,130,231; 6,153,628; 6,160,099; and 6,171,588; and International Publication Nos. WO 95/22543; WO 98/33919; WO 00/78815; and WO 02/070007, each of which is incorporated herein by reference in its entirety. In a certain embodiment, the anti-angiogenic agent is VITAXIN™ (Medlmmune, Inc.) or an antigen-binding fragment thereof.

In a specific embodiment, an anti-angiogenic agent is endostatin. Naturally occurring endostatin consists of the C-terminal ˜180 amino acids of collagen XVIII (cDNAs encoding two splice forms of collagen XVIII have GenBank Accession Nos. AF18081 and AF18082). In another embodiment, an anti-angiogenic agent is a plasminogen fragment (the coding sequence for plasminogen can be found in GenBank Accession Nos. NM_{—}000301 and A33096). Angiostatin peptides naturally include the four kringle domains of plasminogen, kringle 1 through kringle 4. It has been demonstrated that recombinant kringle 1, 2 and 3 possess the anti-angiogenic properties of the native peptide, whereas kringle 4 has no such activity (Cao et al., 1996, J. Biol. Chem. 271:29461-29467). Accordingly, the angiostatin peptides comprises at least one and preferably more than one kringle domain selected from the group consisting of kringle 1, kringle 2 and kringle 3. In a specific embodiment, the anti-angiogenic peptide is the 40 kDa isoform of the human angiostatin molecule, the 42 kDa isoform of the human angiostatin molecule, the 45 kDa isoform of the human angiostatin molecule, or a combination thereof. In another embodiment, an anti-angiogenic agent is the kringle 5 domain of plasminogen, which is a more potent inhibitor of angiogenesis than angiostatin (angiostatin comprises kringle domains 1-4). In another embodiment, an anti-angiogenic agent is antithrombin III. Antithrombin III, which is referred to hereinafter as antithrombin, comprises a heparin binding domain that tethers the protein to the vasculature walls, and an active site loop which interacts with thrombin. When antithrombin is tethered to heparin, the protein elicits a conformational change that allows the active loop to interact with thrombin, resulting in the proteolytic cleavage of said loop by thrombin. The proteolytic cleavage event results in another change of conformation of antithrombin, which (i) alters the interaction interface between thrombin and antithrombin and (ii) releases the complex from heparin (Carrell, 1999, Science 285:1861-1862, and references therein). O'Reilly et al. (1999, Science 285:1926-1928) have discovered that the cleaved antithrombin has potent anti-angiogenic activity. Accordingly, in one embodiment, an anti-angiogenic agent is the anti-angiogenic form of antithrombin. In another embodiment, an anti-angiogenic agent is the 40 kDa and/or 29 kDa proteolytic fragment of fibronectin.

In another embodiment, an anti-angiogenic agent is a urokinase plasminogen activator (uPA) receptor antagonist. In one mode of the embodiment, the antagonist is a dominant negative mutant of uPA (see, e.g., Crowley et al., 1993, Proc. Natl. Acad. Sci. USA 90:5021-5025). In another mode of the embodiment, the antagonist is a peptide antagonist or a fusion protein thereof (Goodson et al., 1994, Proc. Natl. Acad. Sci. USA 91:7129-7133). In yet another mode of the embodiment, the antagonist is a dominant negative soluble uPA receptor (Min et al., 1996, Cancer Res. 56:2428-2433). In another embodiment, a therapeutic molecule is the 16 kDa N-terminal fragment of prolactin, comprising approximately 120 amino acids, or a biologically active fragment thereof (the coding sequence for prolactin can be found in GenBank Accession No. NM_{—}000948). In another embodiment, an anti-angiogenic agent is the 7.8 kDa platelet factor-4 fragment. In another embodiment, a therapeutic molecule is a small peptide corresponding to the anti-angiogenic 13 amino acid fragment of platelet factor-4, the anti-angiogenic factor designated 13.40, the anti-angiogenic 22 amino acid peptide fragment of thrombospondin I, the anti-angiogenic 20 amino acid peptide fragment of SPARC, the small anti-angiogenic peptides of laminin, fibronectin, procollagen, or EGF, or small peptide antagonists of integrin α_{V}β₃ or the VEGF receptor. In another embodiment, the small peptide comprises an RGD or NGR motif. In certain embodiments, an anti-angiogenic agent is a TNF-α antagonist. In other embodiments, an anti-angiogenic agent is not a TNF-α antagonist.

Nucleic acid molecules encoding proteins, polypeptides, or peptides with anti-angiogenic activity, or proteins, polypeptides or peptides with anti-angiogenic activity can be administered to a subject at risk of or with a non-neoplastic hyperproliferative epithelial and/or endothelial cell disorder in accordance with the methods described herein. Further, nucleic acid molecules encoding derivatives, analogs, fragments, or variants of proteins, polypeptides, or peptides with anti-angiogenic activity, or derivatives, analogs, fragments, or variants of proteins, polypeptides, or peptides with anti-angiogenic activity can be administered to a subject at risk of or with a non-neoplastic hyperproliferative epithelial and/or endothelial cell disorder in accordance with the methods. In specific embodiments, such derivatives, analogs, variants, and fragments retain the anti-angiogenic activity of the full-length, wild-type protein, polypeptide, or peptide.

Proteins, polypeptides, or peptides that can be used as anti-angiogenic agents can be produced by any technique well-known in the art or described herein. Proteins, polypeptides or peptides with anti-angiogenic activity can be engineered so as to increase the in vivo half-life of such proteins, polypeptides, or peptides utilizing techniques well-known in the art or described herein. In certain embodiments, anti-angiogenic agents that are commercially available are used in the compositions and methods. The anti-angiogenic activity of an agent can be determined in vitro and/or in vivo by any technique well-known to one skilled in the art.

5.2.3.3 Neuroprotective Agents

Neuroprotective agents are well known in the art and can be compounds which prevent or delay the death of neuronal cells. As non-limiting examples, neuroprotective agents, which can be administered either alone or in combination with an EphB receptor binding peptide and/or EphB receptor binding compound, can be anti-apoptotic compounds such as small molecule drugs, peptides, proteins, antibodies or a combination thereof. Neuroprotective agents may act through interference with one or more apoptotic or necrotic pathways, activation of neural growth hormone receptors or modulation of ion channels. One skilled in the art understands that these and additional neuroprotective agents described herein or known in the art can be useful as therapeutic agents.

5.3 Biological Assays

The protocols and compositions are tested in vitro, and then in vivo, for the desired activity, prior to use in humans. Various in vitro assays that are well known to those skilled in the art can be used to test or determine the biological activity or properties of an EphB receptor binding peptide or EphB receptor binding compound identified by the methods described herein. For example, surface plasmon resonance (e.g., a BIAcore assay) can be used to determine the binding affinity constants (e.g., Kₐ, K_{d}, Kₒₙ, and K_{off}) of an EphB receptor binding peptide or an EphB receptor binding compound for one or more EphB receptors. Non-limiting examples of assays that can be used to measure the K_{d} and/or IC₅₀ to characterize the binding affinity of EphB receptor binding peptides and EphB receptor binding compounds include ELISA, isothermal titration calorimetry, and fluorescent polarization assay. For ELISA assays to measure affinity constants (e.g., Kₐ, K_{d}, Kₒₙ, and K_{off}) EphB receptors are immobilized on a surface and various concentrations of EphB receptor binding peptides or EphB receptor binding compounds are tested. For ELISA assays to measure the IC₅₀ value wherein EphB receptor binding peptides and EphB receptor binding compounds can inhibit the binding of an EphB receptor to an Ephrin B ligand, either the EphB receptor or the Ephrin B ligand can be immobilized on a surface. Subsequently, various concentrations of EphB receptor binding peptides or EphB receptor binding compounds are tested to determine the concentration at which 50% inhibition of the binding of the EphB receptor and the Ephrin B ligand is detected. In specific embodiments, EphB receptors are immobilized on a surface and the ability of EphB receptor binding peptides or EphB receptor binding compounds to inhibit binding of soluble Ephrin B ligand to the EphB receptor is tested. In certain embodiments, Ephrin B ligands are immobilized on a surface and the ability of EphB receptor binding peptides or EphB receptor binding compounds to inhibit binding of soluble EphB receptors to immobilized Ephrin B ligand is tested. In specific embodiments, the concentration of soluble Ephrin B ligand or EphB receptor used in the assay to measure the IC₅₀ value is approximately 0.001 μM, 0.005 μM, 0.01 μM, 0.05 μM, 0.1 μM, 0.5 μM, 1 μM, 10 μM, 20 μM, 30 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1000 μM, or 5000 μM. Specific parameters that may affect the measurement of the K_{d} and IC₅₀ values include, but are not limited to the following parameters, species of EphB receptor or Ephrin B ligand, purity of the EphB receptor binding peptide or EphB receptor binding compound preparations, and sensitivity of the detection agent.

Isothermal titration calorimetry (ITC) can be performed with a Microcal MCS ITC at 25° C. Titrations are performed following an initial injection by making a series of injections of EphB receptor binding peptides or EphB receptor binding compounds into EphB receptors in the sample cell. Dilution data are fit to a line and subtracted from the corresponding titration data, which are analyzed with Origin ITC Software version 5.0 (Microcal Software Inc.). The curves are fit to a single binding cite model (Wiseman et al., Anal. Biochem., 1989, 179:131-137.

Fluoresent polarization assay can be performed with EphB receptor binding peptides or EphB receptor binding compounds that are conjugated to a fluoresent marker (.e.g, Alexa-532). Serial dilutions of EphB receptors are combined with the labeled EphB receptor binding peptides or EphB receptor binding compounds conjugated to a fluorescent marker in the absence or presence of unconjugated EphB receptor binding peptides or EphB receptor binding compounds as a control for nonspecific binding. After allowing the combined mixture to equilibriate for 30 minutes at room temperature, measurements are taken with a Tecan Genios Pro (Tecan Instrucments) using the appropriate excitation and emission wavelength. The experimental data can be analyzed with Prism software version 4.0 (GraphPad Software Inc., San Diego, Calif.). The dissociation constant (Kd) values can be generated by fitting the experimental data using a one-site binding hyperbola nonlinear regression model.

In vitro assays that can be used to measure receptor movement or clustering on the plasma membrane include, for example, fluorescence microscopy (see, e.g., Dove, 2006, Nature Methods 3:223-229). In vitro assays to measure receptor phosphorylation include protein kinase assays using p-32. Assays to measure degradation include Western blots using antibodies that immunospecifically bind to the target protein (e.g., an EphB receptor of interest).

In vitro assays which can be used to determine whether administration of a specific therapeutic or prophylactic protocol is indicated, include in vitro cell culture assays in which a patient tissue sample is grown in culture, and exposed to or otherwise administered a protocol, and the effect of such protocol upon the tissue sample is observed, e.g., inhibition of cell proliferation or survival. A lower level of proliferation or survival of the contacted cells (e.g., cancer cells, vascular cells, or neuronal cells) indicates that the therapeutic agent is effective to treat the condition in the patient. Alternatively, instead of culturing cells from a patient, therapeutic agents and methods may be screened using cells of a tumor or malignant cell line. Many assays standard in the art can be used to assess such survival and/or growth; for example, cell proliferation can be assayed by measuring ³H-thymidine incorporation, flow cytometry, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, increased clustering, phosphorylation, internalization and/or degradation of an Eph receptor, preferably, an EphB1, EphB2 or EphB4 receptor. Additional assays to determine the effect of a particular composition on cancer cell growth include observing formation of colonies in a three-dimensional substrate such as soft agar or the formation of tubular networks or web-like matrices in a three-dimensional basement membrane or extracellular matrix preparation, such as MATRIGEL™. Non-cancer cells do not form colonies in soft agar and form distinct sphere-like structures in three-dimensional basement membrane or extracellular matrix preparations.

Compounds for use in therapy can be tested in suitable animal model systems prior to testing in humans, including but not limited to in rats, mice, chicken, cows, monkeys, rabbits, hamsters, etc., for example, the animal models. For example, but not by way of limitation, for the study of cancer, the SCID mouse model or transgenic mice where a mouse EphB receptor is replaced with the human EphB receptor, nude mice with human xenografts, or any animal model (including hamsters, rabbits, etc.) known in the art and described in Relevance of Tumor Models for Anticancer Drug Development (1999, eds. Fiebig and Burger); Contributions to Oncology (1999, Karger); The Nude Mouse in Oncology Research (1991, eds. Boven and Winograd); and Anticancer Drug Development Guide (1997 ed. Teicher), each of which is incorporated by reference herein in its entirety, may be used. The compounds can then be used in the appropriate clinical trials.

Toxicity and efficacy of the prophylactic and/or therapeutic protocols described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method, the therapeutically effective dose can be estimated initially from cell culture assays. A dose may be formulated in animal models to achieve a circulating plasma concentration range that includes the ICₛₒ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The invention further provides methods for identifying an IC₅₀ values of the EphB receptor binding compounds, which represents the concentration of the EphB receptor binding compound that is required for 50% inhibition of binding of, for example, binding of an EphrinB ligand to an EphB receptor. Protocols for determining IC₅₀ values are known to one of skill in the art, and are described for example, in Example 6, infra.

The invention further provides assays to determine the efficacy of the EphB receptor binding compounds. For example, to test whether an agonistic EphB receptor binding compound (e.g., a multimeric peptide) is efficacious, the EphB receptor binding compound is contacted with cells from a patient, e.g., cancer cells, and assays are performed to determine the effect of the EphB receptor binding compound on various endpoints, such as proliferation of the cancer cells, EphB receptor clustering, transphosphorylation, internalization and/or degradation. An agonistic EphB receptor binding compound is determined to be efficacious if: (1) there is a decrease or inhibition of proliferation of the cancer cells relative to a control; (2) there is an increase in EphB receptor transphosphorylation relative to a control; (3) there is an increase in EphB receptor clustering relative to a control; (4) there is an increase in EphB receptor internalization relative to a control; or (5) there is an increase in EphB receptor degradation relative to a control. An antagonistic EphB receptor binding compound (e.g., a multimeric peptide) is determined to be efficacious if: (1) there is a decrease or no change of proliferation of the cancer cells relative to a control; (2) there is decrease in EphB receptor transphosphorylation relative to a control; (3) there is a decrease in EphB receptor clustering relative to a control; (4) there is decrease in EphB receptor internalization relative to a control; or (5) there is decrease in EphB receptor degradation relative to a control.

Further, any assays known to those skilled in the art can be used to evaluate the prophylactic and/or therapeutic utility of the combinatorial therapies disclosed herein for treatment or prevention of cancer.

5.4 Compositions and Methods of Administration

The present invention provides compositions comprising one or more EphB receptor binding peptides and/or EphB receptor binding compounds (e.g., multimeric peptides and/or conjugates described herein). Such compositions can further comprise a chemotherapy, a hormonal therapy, a radiation therapy, a biological therapy or an immunotherapy. Such compositions may also further comprise a pharmaceutically acceptable carrier or excipient. The invention also provides methods of preventing, treating or managing an EphB receptor related disease, comprising administering to a subject in need thereof a prophylactically or therapeutically effective amount of one or more EphB receptor binding compounds (e.g., EphB receptor binding peptides and EphB receptor binding compounds described herein). In certain embodiments, the EphB receptor binding compound is the single active agent used in the methods provided herein. In other embodiments, the EphB receptor binding peptide is the single active agent used in the methods provided herein. In one embodiment, the EphB receptor binding compound or EphB receptor binding peptide is not used in combination with another therapy. EphB receptor related diseases to be prevented, treated and/or managed by the methods described herein include but are not limited to, neoplastic diseases, cancers, vascular diseases (e.g., macular degeneration) and neurological disorders (e.g., spinal cord injury).

5.4.1 Pharmaceutical Compositions

In specific embodiments, the invention provides methods of treatment and prophylaxis by administering to a subject an effective amount of an EphB receptor binding peptide and/or EphB receptor binding compound either in combination with a pharmaceutically acceptable carrier. In other specific embodiments, the invention provides pharmaceutical compositions comprising one or more EphB receptor binding peptides and/or EphB receptor binding compounds either alone or in combination with one or more other therapeutic or prophylactic agents useful in the methods described herein.

In a specific embodiment, a pharmaceutical composition comprises a purified EphB receptor binding peptide and/or EphB receptor binding compound (e.g., the EphB receptor binding peptide and/or EphB receptor binding compound is preferably substantially free from substances that limit its effect or produce undesired side-effects).

The compositions include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a therapeutically effective amount of a therapeutic agent disclosed herein (e.g., EphB receptor binding peptide and/or EphB receptor binding compound) and a pharmaceutically acceptable carrier. In specific embodiments, compositions comprise a therapeutically effective amount of one or more one or more proteins and a pharmaceutically acceptable carrier. In a further embodiment, the composition further comprises an additional cancer or non-cancer therapeutic. Preferably, the therapeutic agent(s) in the composition are purified.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete) or MF59C.1 adjuvant available from Chiron, Emeryville, Calif.), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. In specific embodiments, water is a carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of the compositions are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In one embodiment, the compositions are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with monoclonal antibodies, even trace amounts of harmful and dangerous endotoxin must be removed. In particular embodiments, endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

Various delivery systems are known and can be used to administer a therapy, e.g., prophylactic or therapeutic agent, useful for preventing, treating or managing a disease or symptoms thereof, e.g., encapsulation in liposomes, microparticles, microcapsules, recombinant cells capable of expressing the polypeptide fragment, receptor-mediated endocytosis (see, e.g., Wu and Wu, 1987, J. Biol. Chem. 262:4429-4432), construction of a nucleic acid as part of a retroviral or other vector, etc. Methods of administering a therapy, e.g., prophylactic or therapeutic agent, include, but are not limited to, parenteral administration (e.g., intradermal, intramuscular, intraperitoneal, intravenous and subcutaneous), epidural, and mucosal (e.g., intranasal, inhaled, and oral routes). In a specific embodiment, therapies, e.g., prophylactic or therapeutic agents, are administered intramuscularly, intravenously, or subcutaneously. The therapies, e.g., prophylactic or therapeutic agents, may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the therapies, e.g., prophylactic or therapeutic agents, for use in methods described herein locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In yet another embodiment, the therapy, e.g, prophylactic or therapeutic agent, can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the agents (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; International Publication Nos. WO 99/15154 and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a specific embodiment, the polymer used in a sustained release formulation is inert, free of leachable impurities, stable on storage, sterile, and biodegradable. In yet another embodiment, a controlled or sustained release system can be placed in proximity of the therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapies, e.g., prophylactic or therapeutic agents. See, e.g., U.S. Pat. No. 4,526,938; International Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. R^{e1}. Bioact. Mater. 24:853-854; and Lam et al., 1997, Proc. Int'l. Symp. Control R^{e1}. Bioact. Mater. 24:759-760.

5.4.2 Formulations

Pharmaceutical compositions for use in accordance with the present invention may be formulated in conventional manner using one or more physiologically acceptable carriers or excipients.

Thus, the agents for use in the methods and their physiologically acceptable salts and solvates may be formulated for administration by inhalation or insufflation (either through the mouth or the nose) or oral, parenteral or mucosal (such as buccal, vaginal, rectal, sublingual) administration. In a certain embodiment, local or systemic parenteral administration is used.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the therapies, e.g., prophylactic or therapeutic agents, for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The therapies, e.g., prophylactic or therapeutic agents, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The therapies, e.g., prophylactic or therapeutic agents, may also be formulated in rectal compositions such as suppositories or retention enemas, e.g., containing conventional suppository bases such as cocoa butter or other glycerides.

In addition to the formulations described previously, the therapies, e.g., prophylactic or therapeutic agents, may also be formulated as a depot preparation. Such long acting formulations may be administered by implantation (for example subcutaneously or intramuscularly) or by intramuscular injection. Thus, for example, the therapies, e.g., prophylactic or therapeutic agents, may be formulated with suitable polymeric or hydrophobic materials (for example as an emulsion in an acceptable oil) or ion exchange resins, or as sparingly soluble derivatives, for example, as a sparingly soluble salt.

The invention also provides that a therapy, e.g., prophylactic or therapeutic agent, is packaged in a hermetically sealed container such as an ampoule or sachette indicating the quantity. In one embodiment, the therapy, e.g., prophylactic or therapeutic agent, is supplied as a dry sterilized lyophilized powder or water free concentrate in a hermetically sealed container and can be reconstituted, e.g., with water or saline to the appropriate concentration for administration to a subject.

In a specific embodiment, the formulation and administration of various therapies for EphB receptor related diseases, e.g., neoplastic diseases, cancer, vascular diseases (e.g., macular degeneration) or neurological diseases are known in the art and often described in the Physicians' Desk Reference, 61^{st} ed. (2007).

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

5.4.3 Dosage and Frequency of Administration

The amount of a therapy, e.g., prophylactic or therapeutic agent, or a composition which will be effective in the prevention, treatment, and/or management of an EphB receptor related disease, or one or more symptoms thereof can be determined by standard clinical methods. The frequency and dosage will vary also according to factors specific for each patient depending on the specific therapies (e.g., the specific therapeutic or prophylactic agent or agents) administered, the severity of the disorder, disease, or condition, the route of administration, as well as age, body, weight, response, and the past medical history of the patient. For example, the dosage of a prophylactic or therapeutic agent or a composition (e.g., a composition comprising an EphB receptor binding peptide and/or EphB receptor binding compound) which will be effective in the treatment, prevention and/or management of an EphB receptor related disease, or one or more symptoms thereof can be determined by administering the composition to an animal model such as, e.g., the animal models disclosed herein or known in to those skilled in the art. In addition, in vitro assays may optionally be employed to help identify optimal dosage ranges. Suitable regimens can be selected by one skilled in the art by considering such factors and by following, for example, dosages are reported in literature and recommended in the Physicians' Desk Reference (61^{st} ed., 2007).

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). For antibodies, proteins, multimeric peptides, polypeptides, peptides and fusion proteins encompassed by the invention (e.g., an EphB receptor binding peptide and/or EphB receptor binding compound), the dosage administered to a patient is typically 0.0001 mg/kg to 100 mg/kg of the patient's body weight. In particular embodiments, the dosage administered to a patient is between approximately 0.0001 mg/kg and approximately 20 mg/kg, approximately 0.0001 mg/kg and approximately 10 mg/kg, approximately 0.0001 mg/kg and approximately 5 mg/kg, approximately 0.0001 and approximately 2 mg/kg, approximately 0.0001 and approximately 1 mg/kg, approximately 0.0001 mg/kg and approximately 0.75 mg/kg, approximately 0.0001 mg/kg and approximately 0.5 mg/kg, approximately 0.0001 mg/kg to approximately 0.25 mg/kg, approximately 0.0001 to approximately 0.15 mg/kg, approximately 0.0001 to approximately 0.10 mg/kg, approximately 0.001 to approximately 0.5 mg/kg, approximately 0.01 to approximately 0.25 mg/kg or approximately 0.01 to approximately 0.10 mg/kg of the patient's body weight. The dosage and frequency of administration of an EphB receptor binding peptide and/or an EphB receptor binding compound may be reduced by enhancing uptake and tissue penetration of the peptide or compound by modifications such as, for example, lipidation.

In a specific embodiment, the dosage of EphB receptor binding peptides and/or EphB receptor binding compounds administered to prevent, treat, manage, and/or manage an EphB receptor related disease in a patient is 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight. In another embodiment, the dosage of EphB receptor binding peptides and/or EphB receptor binding compounds administered to prevent, treat and/or manage an EphB receptor related disease in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In certain embodiments, a subject is administered one or more doses of an effective amount of one or more EphB receptor binding peptides and/or EphB receptor binding compounds, wherein the an effective amount of said EphB receptor binding peptides and/or EphB receptor binding compounds prevent at least 20% to 25%, at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% of endogenous ligand (e.g., an Ephrin) from binding to its receptor relative to a negative control as determined by assays well known in the art and/or described herein, e.g., ELISA, BIAcore assay, immunofluorescence assay, in vivo imaging assays.

In other embodiments, a subject is administered one or more doses of an effective amount of one or more an EphB receptor binding peptides and/or EphB receptor binding compounds (e.g., a multimeric peptide), wherein the dose of an effective amount achieves a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the EphB receptor binding peptides and/or EphB receptor binding compounds. In yet other embodiments, a subject is administered a dose of an effective amount of one or more EphB receptor binding peptides and/or EphB receptor binding compounds to achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 mg/ml, at least 375 μg/ml, or at least 400 μg/ml of the EphB receptor binding peptides and/or EphB receptor binding compounds and a subsequent dose of an effective amount of one or more EphB receptor binding peptides and/or EphB receptor binding compounds is administered to maintain a serum titer of at least 0.1 μg/ml, 0.5 μg/ml, 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 mg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml. In accordance with these embodiments, a subject may be administered 1, 2, 3, 4, 5, 6, 7, 8, 9, 10, 11, 12 or more subsequent doses.

In a specific embodiment, the invention provides methods of preventing, treating, and/or managing an EphB receptor related disease, or one or more symptoms thereof, said method comprising administering to a subject in need thereof a dose of at least 10 μg, at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, or at least 120 μg of one or more EphB receptor binding peptides and/or EphB receptor binding compounds. In another embodiment, the invention provides a method of preventing, treating, and/or managing an EphB receptor related disease, said methods comprising administering to a subject in need thereof a dose of at least 10 μg, at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, or at least 120 μg of one or more EphB receptor binding peptides and/or EphB receptor binding compounds once every 3 days, once every 4 days, once every 5 days, once every 6 days, once every 7 days, once every 8 days, once every 10 days, once every two weeks, once every three weeks, or once a month.

The present invention provides methods of preventing, treating and/or managing an EphB receptor related disease, said method comprising: (a) administering to a subject in need thereof one or more doses of a prophylactically or therapeutically effective amount of one or more EphB receptor binding peptides and/or EphB receptor binding compounds; and (b) monitoring the plasma level/concentration of the said administered EphB receptor binding peptide and/or EphB receptor binding compound in said subject after administration of a certain number of doses of the EphB receptor binding peptide and/or EphB receptor binding compound. Moreover, in specific embodiments, said certain number of doses is 1, 2, 3, 4, 5, 6, 7, 8, 9, 10, 11, or 12 doses of a prophylactically or therapeutically effective amount one or more EphB receptor binding peptides and/or EphB receptor binding compounds. In specific embodiments, the methods comprise administering one or more therapies, e.g., prophylactic or therapeutic agents, for a period of 1 month, 2 months, 3 months, 4 months, 5 months, 6 months, 7 months, 8 months, 9 months, 10 months, 11 months, or 12 months. In certain embodiments, the methods comprise administering one or more therapies, e.g., prophylactic or therapeutic agents, for a period of 2 weeks, 2 weeks, 3 weeks, 4 weeks, 5 weeks, 6 weeks, 7 weeks, 8 weeks, 9 weeks, or 10 weeks. In some embodiments, the methods comprise administering one or more therapies, e.g., prophylactic or therapeutic agents, for a period of 1 year, 2 years, 3 years, 4 years, 5 years, 6 years, 7 years, 8 years, 9 years, or 10 years.

In a specific embodiment, the invention provides a method of preventing, treating, and/or managing an EphB receptor related disease, or one or more symptoms thereof, said method comprising: (a) administering to a subject in need thereof a dose of at least 10 μg, at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg of one or more EphB receptor binding peptides and/or EphB receptor binding compounds (e.g., a multimeric peptide); and (b) administering one or more subsequent doses to said subject when the plasma level of the EphB receptor binding peptides and/or EphB receptor binding peptides administered in said subject is less than 0.1 μg/ml, less than 0.25 μg/ml, less than 0.5 μg/ml, less than 0.75 μg/ml, or less than 1 μg/ml. In another embodiment, the invention provides a method of preventing, treating, managing, and/or treating an EphB receptor related disease, said method comprising: (a) administering to a subject in need thereof one or more doses of at least 10 μg, at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg of one or more EphB receptor binding peptides and/or EphB receptor binding compounds (e.g., a multimeric peptide); (b) monitoring the plasma level of the administered EphB receptor binding peptides and/or EphB receptor binding compounds in said subject after the administration of a certain number of doses; and (c) administering a subsequent dose of the EphB receptor binding peptides and/or EphB receptor binding compounds when the plasma level of the administered EphB receptor binding peptides and/or EphB receptor binding compounds in said subject is less than 0.1 μg/ml, less than 0.25 μg/ml, less than 0.5 μg/ml, less than 0.75 μg/ml, or less than 1 μg/ml. In particular embodiments, said certain number of doses is 1, 2, 3, 4, 5, 6, 7, 8, 9, 10, 11, or 12 doses of an effective amount of one or more EphB receptor binding peptides and/or EphB receptor binding compounds.

In certain embodiments, EphB receptor binding peptides and/or EphB receptor binding compounds (e.g., multimeric peptides) are formulated at 1 mg/ml, 5 mg/ml, 10 mg/ml, and 25 mg/ml for intravenous injections and at 5 mg/ml, 10 mg/ml, and 80 mg/ml for repeated subcutaneous administration and intramuscular injection.

For EphB receptor binding peptides and/or EphB receptor binding compounds, the dosage administered to a patient is typically 0.01 mg/kg to 0.1 mg/kg, or 0.1 mg/kg to 100 mg/kg of the patient's body weight. In specific embodiments, the dosage administered to a patient is between approximately 0.1 mg/kg and approximately 20 mg/kg of the patient's body weight, or approximately 1 mg/kg to approximately 10 mg/kg of the patient's body weight.

Effective doses may be extrapolated from dose-response curves derived animal model test systems. In certain exemplary embodiments, the dosage ranges are 0.001-fold to 10.000-fold of the murine LD₅₀, 0.01-fold to 1.000-fold of the murine LD₅₀, 0.1-fold to 500-fold of the murine LD₅₀, 0.5-fold to 250-fold of the murine LD₅₀, 1-fold to 100-fold of the murine LD₅₀, and 5-fold to 50-fold of the murine LD₅₀. In certain specific embodiments, the dosage ranges are 0.001-fold, 0.01-fold, 0.1-fold, 0.5-fold, 1-fold, 5-fold, 10-fold, 50-fold, 100-fold, 200-fold, 500-fold, 1,000-fold, 5,000-fold or 10,000-fold of the murine LD₅₀.

In specific embodiments, the amount of EphB receptor binding peptide and/or EphB receptor binding compound that is effective to activate or inactivate downstream signaling events includes concentrations of at least 0.05 μM, at least 0.1 μM, at least 0.2 at least 0.3 μM, at least 0.4 μM, at least 0.5 μM, at least 0.6 μM, at least 0.7 μM, at least 0.8 μM, at least 0.9 μM, at least 1 μM, at least 5 μM, at least 10 μM, at least 20 μM, at least 30 μM, at least 40 μM, at least 50 μM, at least 60 at least 70 μM, at least 80 μM, at least 90 μM, at least 100 μM or at least 200 μM. Determination of other effective concentrations not described herein can be readily determined by one of ordinary skill in the art.

Therapies (e.g., prophylactic or therapeutic agents), other than EphB receptor binding peptides and/or EphB receptor binding compounds, which have been or are currently being used to prevent, treat and/or manage EphB receptor related diseases can be administered in combination with one or more EphB receptor binding peptides and/or EphB receptor binding compounds according to the methods described herein to prevent, treat and/or manage an EphB receptor related disorder. In a specific embodiment, the dosages of prophylactic or therapeutic agents used in combination therapies are lower than those which have been or are currently being used to prevent, treat and/or manage an EphB receptor related disease. The recommended dosages of agents currently used for the prevention, treatment and/or management of an EphB receptor related disease can be obtained from any reference in the art including, but not limited to, Hardman et al., eds., 2001, Goodman & Gilman's The Pharmacological Basis Of Basis Of Therapeutics, 10th ed., Mc-Graw-Hill, New York; Physicians' Desk Reference (PDR) 60^{th} ed., 2006, Medical Economics Co., Inc., Montvale, N.J.; Physicians' Desk Reference (PDR) 61^{st} ed., 2007, Medical Economics Co., Inc., Montvale, N.J., which are all incorporated herein by reference in their entireties.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In specific embodiments, two or more therapies are administered within the same patient visit.

In certain embodiments, one or more EphB receptor binding peptides and/or EphB receptor binding compounds and one or more other therapies (e.g., prophylactic or therapeutic agents) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the administration of the same EphB receptor binding peptides and/or EphB receptor binding compounds may be repeated and the administrations may be separated by at least 1 day, 2 days, 3 days, 5 days, 10 days, 15 days, 30 days, 45 days, 2 months, 75 days, 3 months, or at least 6 months. In other embodiments, the administration of the same therapy (e.g., prophylactic or therapeutic agent) other than an antibody may be repeated and the administration may be separated by at least at least 1 day, 2 days, 3 days, 5 days, 10 days, 15 days, 30 days, 45 days, 2 months, 75 days, 3 months, or at least 6 months.

5.5 Kits

The invention provides a pharmaceutical pack or kit comprising one or more containers filled with an EphB receptor binding peptide and/or EphB receptor binding compound. Additionally, one or more other prophylactic or therapeutic agents useful for the treatment of an EphB receptor related disease or other relevant agents can also be included in the pharmaceutical pack or kit. In certain embodiments, the other prophylactic or therapeutic agent is an immunomodulatory agent (e.g., anti-Eph receptor antibody). The invention also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration. The invention further provides a diagnostic pack or kit comprising one or more containers filled with an EphB receptor binding peptide and/or EphB receptor binding compound for diagnosing and/or monitoring an EphB receptor related disease. The diagnostic pack or kit may further comprise detection agents for diagnosing and/or monitoring an EphB receptor related disease.

5.6 Articles of Manufacture

The present invention also encompasses a finished packaged and labeled pharmaceutical product. This article of manufacture includes the appropriate unit dosage form in an appropriate vessel or container such as a glass vial or other container that is hermetically sealed. The pharmaceutical product may be formulated in single dose vials as a sterile liquid that contains 10 mM histidine buffer at pH 6.0 and 150 mM sodium chloride. Each 1.0 mL of solution may contain 100 mg of protein, 1.6 mg of histidine and 8.9 mg of sodium chloride in water for injection. In the case of dosage forms suitable for parenteral administration the active ingredient, e.g., an EphB receptor binding peptide and/or an EphB receptor binding compound, is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, intransal, or topical delivery.

In a specific embodiment, the unit dosage form is suitable for intravenous, intramuscular, intranasal, oral, topical or subcutaneous delivery. Thus, the invention encompasses solutions, preferably sterile, suitable for each delivery route.

As with any pharmaceutical product, the packaging material and container are designed to protect the stability of the product during storage and shipment. Further, the products include instructions for use or other informational material that advise the physician, technician or patient on how to appropriately prevent or treat the disorder in question. In other words, the article of manufacture includes instruction means indicating or suggesting a dosing regimen including, but not limited to, actual doses, monitoring procedures, total lymphocyte, mast cell counts, T cell counts, IgE production, and other monitoring information.

Specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material, wherein said pharmaceutical agent comprises an EphB receptor binding compound (e.g., a multimeric peptide) and wherein said packaging material includes instruction means which indicate that said EphB receptor binding compound can be used to prevent, manage, treat, and/or ameliorate one or more symptoms associated with an EphB receptor related disease, or one or more symptoms thereof by administering specific doses and using specific dosing regimens as described herein.

The invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material, wherein one pharmaceutical agent comprises an EphB receptor binding peptide and/or EphB receptor binding compound and the other pharmaceutical agent comprises a second, different therapy (e.g., a different prophylactic or therapeutic agent), and wherein said packaging material includes instruction means which indicate that said agents can be used to treat, prevent and/or ameliorate an EphB receptor related disease, or one or more symptoms thereof by administering specific doses and using specific dosing regimens as described herein.

The invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material, wherein one pharmaceutical agent comprises an EphB receptor binding peptide and/or EphB receptor binding compound and the other pharmaceutical agent comprises a prophylactic or therapeutic agent other than an EphB receptor binding peptide and/or EphB receptor binding compound, and wherein said packaging material includes instruction means which indicate that said agents can be used to treat, prevent and/or ameliorate one or more symptoms associated with an EphB receptor related disease by administering specific doses and using specific dosing regimens as described herein.

The present invention provides that the adverse effects that may be reduced or avoided by the methods described herein are indicated in informational material enclosed in an article of manufacture for use in preventing, treating and/or managing one or more symptoms associated with an EphB receptor related disease. Adverse effects that may be reduced or avoided by the methods described herein include, but are not limited to, vital sign abnormalities (fever, tachycardia, bardycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, and vasodilatation.

Further, the information material enclosed in an article of manufacture for use in preventing, treating, and/or managing an EphB receptor related disease can indicate that foreign proteins may also result in allergic reactions, including anaphylaxis, or cytosine release syndrome. The information material should indicate that allergic reactions may exhibit only as mild pruritic rashes or they may be severe such as erythroderma, Stevens-Johnson syndrome, vasculitis, or anaphylaxis. The information material should also indicate that anaphylactic reactions (anaphylaxis) are serious and occasionally fatal hypersensitivity reactions. Allergic reactions including anaphylaxis may occur when any foreign protein is injected into the body. They may range from mild manifestations such as urticaria or rash to lethal systemic reactions. Anaphylactic reactions occur soon after exposure, usually within 10 minutes. Patients may experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, or eosinophilia.

5.7 Other Utility

The EphB receptor binding peptides and EphB receptor binding compounds described herein are useful in vitro as unique tools for understanding the biological role of Eph receptors (e.g., EphB receptors), including the evaluation of the many factors thought to influence, and be influenced by, the production of Ephrin ligands (e.g., EphrinB ligands) and the receptor binding process. The EphB receptor binding peptides and EphB receptor binding compounds are also useful in the development of other compounds that bind to and activate Eph receptors, because the EphB receptor binding peptides and EphB receptor binding compounds provide important information on the relationship between structure and activity to facilitate such development.

The EphB receptor binding peptides and EphB receptor binding compounds are also useful as competitive binders in assays to screen for new Eph receptor agonists. In such assay embodiments, the EphB receptor binding peptides and EphB receptor binding compounds described herein can be used without modification or can be modified in a variety of ways; for example, by labeling, such as covalently or non-covalently joining a moiety which directly or indirectly provides a detectable signal. In any of these assays, the materials thereto can be labeled either directly or indirectly. Possibilities for direct labeling include label groups such as: radiolabels such as ¹²⁵I, enzymes (U.S. Pat. No. 3,645,090) such as peroxidase and alkaline phosphatase, and fluorescent labels (U.S. Pat. No. 3,940,475) capable of monitoring the change in fluorescence intensity, wavelength shift, or fluorescence polarization. Possibilities for indirect labeling include biotinylation of one constituent followed by binding to avidin coupled to one of the above label groups. The EphB receptor binding peptides and EphB receptor binding compounds may also include spacers or linkers in cases where the EphB receptor binding peptides and EphB receptor binding compounds are to be attached to a solid support.

Nuclear magnetic resonance (NMR) spectroscopy is known for its ability to characterize macromolecular structures, and is a technique for investigating both static and transient features of ligand binding to a target molecule (Pellecchia, et al. 2002 Nature Rev Drug Disc 1:211). NMR spectroscopy is a useful tool for determining the binding of ligands to target molecules, and has the advantage of being able to detect and quantify interactions with high sensitivity without requiring prior knowledge of protein function. Furthermore, NMR spectroscopy can provide structural information on both the target and the ligand to aid subsequent optimization of weak-binding hits into high-affinity leads.

Methods of detecting binding of a ligand compound to a target biomolecule by generating first and second nuclear magnetic resonance correlation spectra from target biomolecules which have been uniformly labeled are reported in U.S. Pat. Nos. 5,698,401 and 5,804,390. The first spectrum is generated from data collected on the target substance in the absence of ligands, and the second in the presence of one or more ligands. A comparison of the two spectra permits determination of which compounds in the mixture of putative ligands bind(s) to the target biomolecule.

EphB receptors may be selectively labeled by incorporation of ¹H, ¹³C, ¹⁵N and/or ¹⁹F into the side chain of one or more amino acid residues. Selectively labeled complexes of an Eph receptor bound to an Eph receptor binding ligand can be exposed to a second molecule and any molecular interaction can be examined by NMR spectroscopy. For example, 2D 13C,1H-HMQC (heteronuclear multiple quantum coherence) and 13C-edited 1H,1H-NOESY NMR experiments can be used to detect molecular interaction and to determine the dissociation constant for any complex. In addition, a predictive model can be created based on the three-dimensional structure of the target and from the relative position of the ligand with respect to the labeled side chain. The use of several different labeled side-chains in a single, selectively-labeled, target-molecule will improve the resolution as well as the predictive nature of the model.

Because non-peptidic small molecules may be more suitable than peptides for clinical development, high throughput screening can be used to screen chemical libraries for small molecules that disrupt or prevent formation of the Eph-Ephrin complex. The assay uses immobilized Eph receptor ectodomains in complex with Ephrin-alkaline phosphatase fusion proteins. The ability to decrease bound alkaline phosphatase activity will identify small molecule inhibitors of the Eph-Ephrin interaction.

Moreover, based on their ability to selectively bind to EphB receptors, the EphB receptor binding peptides and EphB receptor binding compounds described herein can be used as reagents for selectively detecting EphB receptors on living cells, fixed cells, in biological fluids, in tissue homogenates, in purified, natural biological materials, etc. For example, by labeling peptides described herein, one can selectively identify cells having receptors such as EphB1, EphB2, EphB3 or EphB4 on their surfaces. In addition, based on their ability to bind Eph receptors, the peptides can be used in in situ staining, FACS (fluorescence-activated cell sorting), Western blotting, ELISA, etc. In addition, based on their ability to selectively bind EphB receptors, the peptides can be used in receptor purification, or in purifying cells expressing only specific Eph receptors on the cell surface (or inside permeabilized cells).

The EphB receptor binding peptides and EphB receptor binding compounds described herein can also be utilized as commercial reagents for various medical research and diagnostic uses. Such uses include but are not limited to: (1) use as a calibration standard for quantitating the activities of candidate EphB agonists in a variety of functional assays; (2) use to maintain the proliferation and growth of EphB-dependent cell lines; (3) use in structural analysis of the EphB-receptor ligand-binding interfaces through co-crystallization; (4) use to investigate the mechanism of EphB signal transduction/receptor activation; (5) other research and diagnostic applications wherein the EphB receptor is activated or such activation is conveniently calibrated against a known quantity of an EphB agonist, and the like; and (6) other research and diagnostic applications wherein the EphB receptor is inhibited or such inhibition is conveniently calibrated against a known quantity of an EphB antagonist, and the like. In certain embodiments, the EphB receptor binding peptides and EphB receptor binding compounds described herein can be used to diagnose an EphB receptor related disease in a subject. In some embodiments, EphB receptor binding compounds can be used to purge cells from a patient sample. In specific embodiments, EphB receptor binding peptides and EphB receptor binding compounds described herein can be used to monitor the progression of an EphB receptor related disease in a subject. For example, EphB receptor binding peptides and/or EphB receptor binding compounds that are conjugated to a detectable marker or imaging agent can be administered to a subject to identify tissues that comprise aberrant expression of EphB receptor. Computed tomography (CT), positron emitted tomography (PET), magnetic resonance imaging (MRI) and other radiological imaging techniques are well known in the art of medical diagnostics and other imaging based applications.

In particular embodiments, methods of detecting or diagnosing an EphB receptor related disorder in a subject comprises contacting a sample (i.e., cells, tissues, biological fluids, biological materials) of said subject with an isolated conjugate comprising an EphB receptor binding peptide having a length of between approximately 5 to approximately 50 amino acid residues and a detectable agent, and wherein the isolated conjugate selectively binds to a member of the EphB receptor family. Methods and techniques for in vivo and in vitro techniques are discusses, e.g., in U.S. Pat. No. 6,572,856, which is incorporated by reference herein in its entirety. In other embodiments, the invention also provides for methods for detecting aberrant expression of an EphB receptor in a subject, the methods comprising (a) contacting samples or cells of the subject with an isolated EphB receptor binding compound comprising a detectable agent; and (b) detecting binding of the isolated EphB receptor binding compound to the samples or cells of the subject, wherein aberrant expression of the EphB receptor is detected if the binding of the isolated EphB receptor binding compound to said samples of the subject is higher or lower than the binding of the isolated EphB receptor binding compound to control samples or cells that have normal expression of the EphB receptor. In specific embodiments, the level of EphB receptor expression is measured in a subject and is compared to the level of EphB receptor expression of a healthy subject or a subject who does not have a detectable EphB receptor related disease or to a predetermined reference range for a healthy subject or a subject who does not have a detectable EphB receptor related disease. In certain embodiments, a normal level of EphB receptor expression is the level of EphB receptor expression in a healthy subject or a subject who does not have a detectable EphB receptor related disease. In other embodiments, methods of detecting aberrant expression of an EphB receptor in a subject comprise measuring the level of EphB receptor expression in a sample of the subject or in the subject and comparing the level of EphB receptor expression in the sample or the subject to the level of expression of a healthy subject or a subject who does not have a detectable EphB receptor related disease or to a predetermined reference range for a subject that aberrantly expresses an EphB receptor or has an EphB receptor related disease, wherein aberrant expression of an EphB receptor is detected if there is an equivalent or greater level of EphB receptor expression in the sample or the subject relative to the level of EphB receptor expression of a healthy subject or a subject who does not have a detectable EphB receptor related disease or to the predetermined reference range. In certain embodiments, methods of detecting aberrant expression of an EphB receptor is for the purpose of diagnosing an EphB receptor related disease. In other embodiments, methods of detecting aberrant expression of an EphB receptor is for the purpose of monitoring the progression of an EphB receptor related disease or for the purpose of monitoring the effectiveness of a therapy.

### 6. EXAMPLES

The following non-limiting examples illustrate the synthesis, analysis and/or utility of certain EphB receptor binding peptides and EphB receptor binding compounds of the present invention.

### Example 1

### PEG-Peptide Conjugation

PEG which has been activated such that it can react with amino groups of peptides and proteins is commercially available (e.g., from Nektar Therapeutics, Huntsville Ala.). The linking of the amino group of a peptide to PEG of a specified size is termed amine PEGylation, and can result in an improved serum half-life profile for the peptide. The PEGylation of the TNYL-RAW peptide (SEQ ID NO:39, Table 1) involves a chemical coupling reaction between the amino group of a peptide and a monofunctional activated PEG to produce a physiologically stable amide linkage. An activated PEG which is commonly used in this type of reaction is the monomethoxy-N-hydroxysuccinimide ester of PEG carboxylic acid (MePEG-NHS). The reaction is carried out by combining 1-10 molar equivalents of Me-PEG NHS per mole of the TNYL-RAW peptide, at a solution concentration of 0.5 to 5 mg/ml peptide. The coupling is performed at a pH between 7-9 and at a temperature between 4-25° C., for a total reaction time of between 15-120 min. The PEG-conjugated peptide is subsequently isolated and characterized.

### Example 2

### Conjugation of Peptides to Bifunctional PEGs

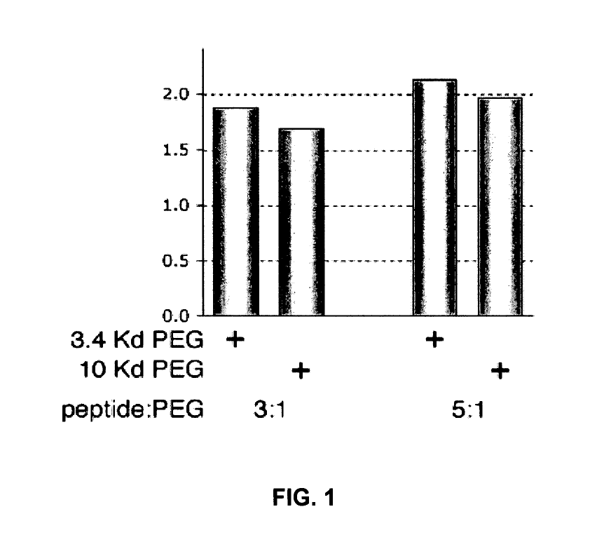

The conjugation reaction described above may be employed with a multifunctional PEG in order to link two or more peptides together to form a PEG-linked multimeric peptide. The TNYL-RAW-biotin peptide, reconstituted in phosphate buffer solution (PBS), was reacted with SMB-PEG-SMB, a bifunctional linear PEG activated at both terminal ends with succinimidyl α-methylbutanoate groups. The reaction was conducted by reconstituting the SMB-PEG-SMB with PBS to 6-11 mM, followed by mixing 100 μl of TNYL-RAW-biotin (260 μM) with reconstituted SMB-PEG-SMB. The reaction was performed using two different sizes of SMB-PEG-SMB reactant, 3.4 kDa and 10 kDa. The reaction was repeated using two different peptide:PEG molar ratios, 3:1 and 5:1. For the 3:1 ratio, 260 μM TNYL-RAW was combined with 87 μM SMB-PEG-SMB in a total volume of approximately 100 μl. For the 5:1 ratio, 260 μM TNYL-RAW was combined with 52 μM SMB-PEG-SMB in a total volume of approximately 1004 The reactants were incubated for 30 min at ambient temperature, then at 4° C. overnight. The reactions were dialyzed against 2-3 changes of 500 ml PBS for 24 h at 4° C. The reactions were recovered from dialysis units and characterized by optical absorption (OD₂₈₀) to determine peptide concentration. The results are shown in FIG. 1. The results indicate that the products contain approximately 2 peptides per PEG linker molecule, suggesting that peptide dimers were produced.

### Example 3

### TNYL-RAW Peptide Coupled to PEG Binds to EphB4

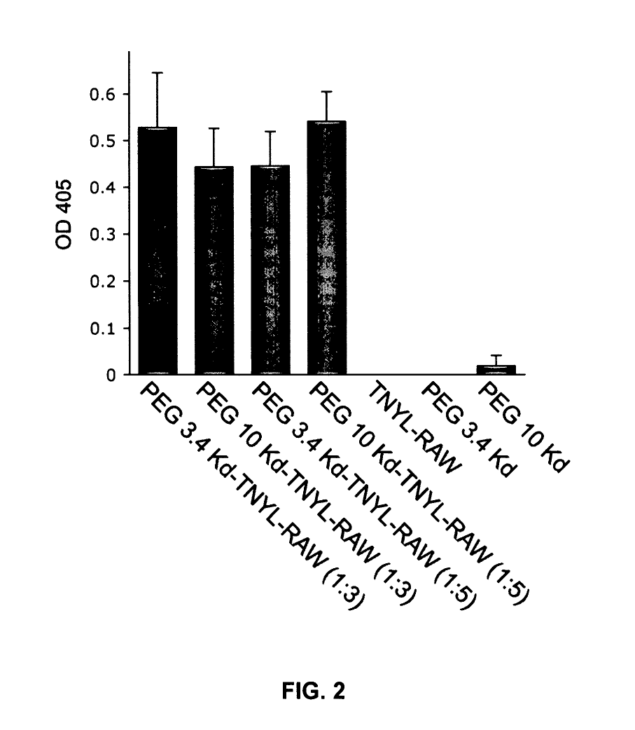

An experiment was designed to evaluate whether PEG-TNYL-RAW-biotin captured on wells coated with anti-PEG antibodies would bind to murine EphB4 AP. Reacti-bind Protein L coated plates were washed three times with TBST/Ca (described, supra). Plates were coated with AGP3 (mouse anti-PEG IgM) 1 μg/ml diluted in TBST/Ca (100 μl/well) for 1.5 h at ambient temperature. The plates were washed four times with TBST/Ca. The products of several PEGylation reactions performed (as described supra) with different PEG sizes at different PEG:peptide starting ratios were added to the plates in 40 μl total TBST/Ca. The PEGylation products included: PEG 3.4 kDa-TNYL-RAW (1:3 PEG:peptide); PEG 10 kDa-TNYL-RAW (1:3 PEG:peptide); PEG 3.4 kDa-TNYL-RAW (1:5 PEG:peptide); PEG 10 kDa-TNYL-RAW (1:5 PEG:peptide). Controls (TNYL-RAW-biotin; SMB-PEG-SMB 3.4 kDa; and SMB-PEG-SMB 10 kDa) were added at the same concentration as the PEGylation reaction products. The plates were incubated for 1.5 h at ambient temperature, then washed four times with TBST/Ca. Substrate, 1 mg/ml PNPP in SEAP buffer, was added in the amount of 100 μl/well (substrate was prepared as described supra). Optical density analysis (OD405) was performed using a plate reader to demonstrate that PEG-TNYL-RAW binds to EphB4. Results are shown in FIG. 2.

### Example 4

### The Effect of Amino Acid Sequence Modification on Binding Affinity to the EphB4 Receptor

An experiment was performed to assess the importance of certain amino acids of the TNYLFSPNGPIARAW peptide (“TNYL-RAW”; SEQ ID NO:39 in Table 1) to its EphB4 binding affinity. Specifically, the importance of the first three amino acids of the TNYL-RAW peptide (SEQ ID NO:39 in Table 1) to the EphB4 binding affinity was evaluated. The experiment, which was performed as detailed below, demonstrated that EphB4 binding affinity is not significantly altered upon removal of either the first amino acid or the first two amino acids of the TNYL-RAW peptide.

The synthetic peptides TNYL-RAW, NYL-RAW, YL-RAW and L-RAW (corresponding to SEQ ID NOS:39, 40, 41 and 42, respectively, in Table 1) were synthesized by Biopeptide (San Diego, Calif.) and tested for their ability to inhibit binding of alkaline phosphatase-tagged ephrin-B2 (ephrin-B2-AP) to EphB receptors. Wells coated with the murine EphB4 ectodomain were incubated with murine ephrin-B2 AP and different concentrations of the indicated peptides. Ephrin-B2 AP binding to the immobilized EphB4 receptor ectodomain was measured in the presence of various concentrations of the peptides and detected by measuring alkaline phosphatase activity. Ephrin-B2 AP binding in the presence of peptide was normalized to binding in the absence of peptide.

Of the peptides tested for their ability to inhibit ephrin binding to murine EphB4, TNYL-RAW, NYL-RAW and YL-RAW each exhibited effective binding inhibition, while the L-RAW peptide did not inhibit ephrin-B2 AP binding as effectively. The concentration of peptides necessary to inhibit binding of the dimeric ephrin-B2 AP by 50% (IC₅₀) was approximately 40 μM for TNYL-RAW, NYL-RAW and YL-RAW. In contrast, the IC₅₀ was determined to be greater than 10 μM for L-RAW.

### Example 5

Stability of TNYL-RAW Peptide in Cell Culture Medium

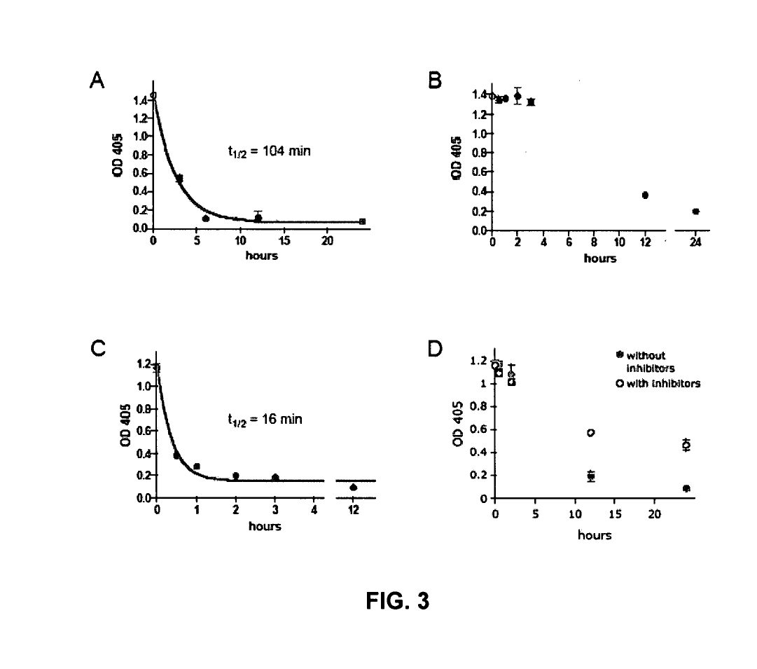

The stability of the TNYL-RAW peptide (SEQ ID NO:39) in cell culture medium was assessed. The TNYL-RAW peptide was synthesized with a biotin tag by the Burnham Institute (La Jolla, Calif.). Biotinylated TNYL-RAW peptide was added at a concentration of 1 μM to PC3 prostate cancer cells cultured overnight and functional (EphB4- and streptavidin-binding) peptides remaining in the cell culture medium at various time points were captured in ELISA plates coated with neutravidin (FIG. 3A). The bound TNYL-RAW peptide was detected with EphB4 conjugated to alkaline phosphatase (AP). The half-life of the TNYL-RAW peptide in cell culture medium with PC3 cells is approximately 104 minutes (FIG. 3A). FIG. 3B shows data from an experiment carried out essential as described for FIG. 3A except that the cell culture medium was replaced with fresh medium just before adding the peptide. The peptide remained intact for several hours and was more stable relative to the peptide in the experiment where the cell culture medium was not replace with fresh medium prior to addition of the peptide. FIG. 3C presents data obtained from an experiment where the TNYL-RAW peptide is incubated in cell conditioned medium in the absence of the cells. The peptide was also rapidly lost (FIG. 3C). The results of FIGS. 3B and 3C suggest that the cells are secreting proteases that degrade the peptide into the cell culture medium. Thus, a mixture of protease inhibitors (including aprotinin, PMSF, leupeptin, and pepstatin) added to cell conditioned medium, with which the peptide was incubated, was able to reduce peptide instability (FIG. 3D).

### Example 6

Dimerization of the TNYL-RAW Peptide by Covalent Coupling to Bifunctional PEG Produces Functional PEGylated TNYL-RAW Peptide

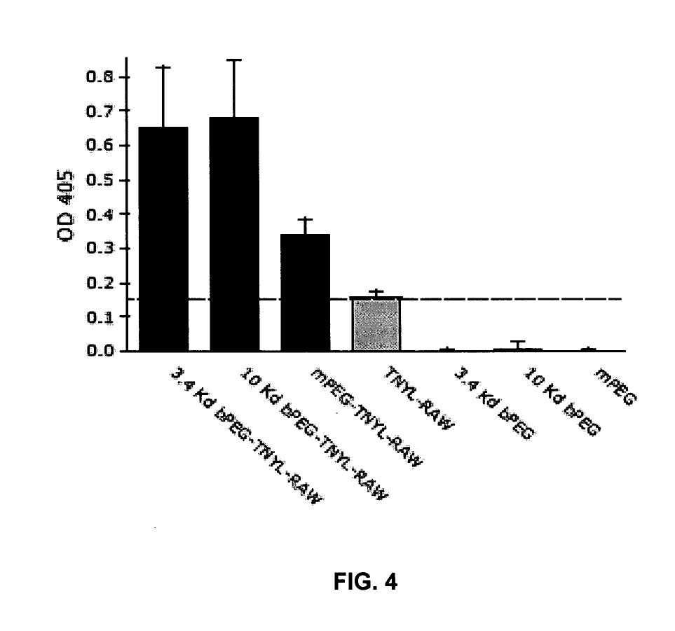

TNYL-RAW peptide (SEQ ID NO:39) synthesized and conjugated to biotin by The Burnhan Institute (La Jolla, Calif.) was coupled to monofunctional PEG (mPEG) (at a ratio of 1:5) or bifunctional PEG (bPEG) of 3.4 kDa or 10 kDa as described in Example 2, supra. To confirm that the PEGylated TNYL-RAW peptide was successfully synthesized, it was captured in ELISA wells coated with anti-PEG antibodies and detected with streptavidin-HRP. The signal from peptide bound to bPEG (bPEG-TNYL-RAW) was higher than the background signal with peptide alone (dashed line) and the signal from peptide coupled to monofunctional PEG (mPEG-TNYL-RAW), demonstrating that the peptide was coupled to the PEG (FIG. 4). The data presented in FIG. 4 is the averages from 2 to 4 measurements with standard deviations. Furthermore, PEGylated TNYL-RAW retained high EphB receptor binding affinity (Table 3) and the ability to inhibit EphB4-ephrin-B2 interaction (Table 4).

The dissociation constants in Table 3 were measured in binding assays as described below. Reacti-bind Protein A coated strip plates were washed three times with the wash buffer TBST/Ca (TBS, 0.01% Tween, 1 mM CaCl₂). Plates were coated with murine EphB4-Fc (reconstituted in PBS 200 μg/ml, diluted to 1 μg/ml in TBST/Ca) 100 μl/well for 1 hour at ambient temperature. The plates were washed four times with TBST/Ca. PEG-TNYL-RAW-biotin (made by The Burnham Institute, La Jolla, Calif.), obtained from the PEGylation reaction described in Example 2 supra, was added at different concentrations in TBST/Ca (100 μl/well) for 1 hour at ambient temperature. The plates were again washed four times with TBST/Ca. Streptavidin-HRP (1:2000) in 100 μl TBST/Ca was added for 1 hour at ambient temperature. The plates were again washed four times with TBST/Ca. To each well was added 100 μl ABTS substrate (i.e., 2,2-azine-bis(3-ethylbenzthiazoline-6-sulfonic acid, the substrate for horseradish peroxidase). (ABTS substrate was prepared by adding 100 mg ABTS to 450 ml 0.05 M filtered (0.22 mm filter) citric acid solution (pH=4), filtering again, storing at 4° C. in foil, then, on day of use, adding 18 μl of 30% H₂O₂ to 10.5 ml ABTS stock solution.) Optical density analysis (OD₄₀₅) was performed using a plate reader to determine the binding affinity of the unconjugated TNYL-RAW peptide and TNYL-RAW peptide conjugated to PEG (3.4 and 10 kDa) to murine EphB4 receptor.

The dissociation constants in Table 3 indicate that the TNYL-RAW peptides conjugated to 3.4 kDa PEG or 10 kDa PEG retained comparable binding affinity to murine EphB4 receptor as the TNYL-RAW peptide not conjugated to PEG.

The IC₅₀ values in Table 4 were obtained from inhibition assays. Murine Ephrin B2-Fc was immobilized on ELISA plates, and binding of EphB4-AP was measured in the presence or absence of the indicated TNYL-RAW peptides conjugated to PEG (3.4 kDa or 10 kDa) as described below. Reati-bind Protein A coated strip plates were washed three times with wash buffer TBST/Ca. Plates were coated with murine ephrin-B2-Fc (reconstituted in PBS 200 μg/ml, diluted to 1 μg/ml with TBST/Ca) 100 μl/well for 1 h at ambient temperature. The control used was Human-Fc (1 μg/ml; 100 μl/well); Human-Fc is the control for ephrin-B2-Fc, which contains the extracellular domain of ephrin-B2 fused to the Fc portion of human IgG1. The plates were washed four times with TBST/Ca. For each well was added: PEG-TNYL-RAW-biotin, from PEGylation reaction in Example 2, supra, at different concentrations; 10 μl murine EphB4-Alkaline Phosphatase (EphB4-AP); and TBST/Ca to 50 μl. Plates were incubates for 3 hours at ambient temperature, then washed four times with TBST/Ca. Substrate, 1 mg/ml PNPP in SEAP buffer, was added in the amount of 100 μl/well. (2×SEAP buffer (pH=9.8) was prepared by: adding 2.1 ml diethanolamine and 10 μl 1M MgCl₂ to 10 ml (total) water; adjusting pH to 9.8; wrapping in foil and storing at ambient temperature; and diluting with water to 1× on day of use). Optical density analysis (OD405) was performed using a plate reader to determine the extent to which PEG-TNYL-RAW-biotin inhibits the EphB4-ephrin-B2 interaction. The control TNYL-RAW peptide was not conjugated to biotin, but the TNYL-RAW peptides conjugated to PEG also contained biotin. Dimer indicates that two TNYL-RAW peptides were conjugated to a PEG molecule, and monomer indicates that one TNYL-RAW peptide was conjugated to a PEG molecule.

TABLE 3
K_{D} Values for TNYL-RAW binding to murine EphB4

| PEG size | K_{D} ± SE* | # experiments |
|---|---|---|
| No PEG | 1.3 ± 0.2 nM | 2 |
| 10 Kd (monomer) | 1.4 ± 0.7 nM | 2 |
| 3.4 Kd (dimer) | 0.9 ± 0.3 nM | 8 |
| 10 Kd (dimer) | 0.9 ± 0.2 nM | 8 |

*based on the estimated concentration of peptide-coupled PEG molecules

TABLE 4
IC₅₀ for Inhibition of murine EphB4/murine ephrin-B2 Binding by
TNYL-RAW

| PEG size | IC₅₀ ± SE* | | # experiments |
|---|---|---|---|
| No PEG | 16 | nM | 1 |
| 10 Kd (monomer) | 21 ± 5 | nM | 2 |
| 3.4 Kd (dimer) | 7 ± 4 | nM | 3 |
| 10 Kd (dimer) | 9 ± 2 | nM | 5 |

*based on the estimated concentration of peptide-coupled PEG molecules

### Example 7

### Dimerization of TNYL-RAW Peptide by Fusion to Human Fc

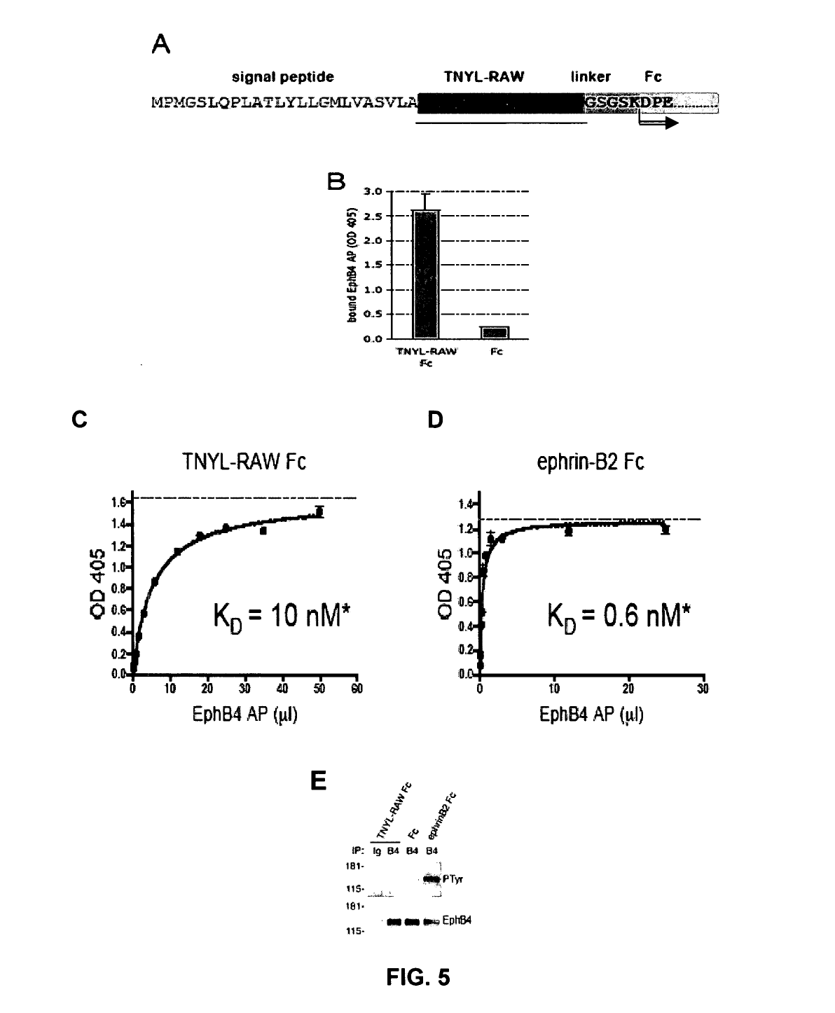

Fusing the TNYL-RAW peptide to human Fc portion of IgG₁ was another approach taken to dimerize the TNYL-RAW peptide and improve stability. A synthetic DNA sequence encoding the TNYL-RAW peptide was cloned into an expression vector. The cloned vector produces the peptide preceded by a signal sequence for secretion into the medium (this signal sequence is cleaved in the mature TNYL-RAW Fc protein) and followed by an intervening GSGSK (SEQ ID NO:76) linker and human Fc (FIG. 5A). Two of the TNYL-RAW Fc fusion proteins dimerize via the Fc domain to produced a molecule comprising two TNYL-RAW peptides and two Fc domains.

The encoded TNYL-RAW Fc fusion protein purified from HEK293 cell culture supernatants were immobilized on protein A ELISA plates and assayed for its ability to bind murine EphB4 conjugated to AP (“EphB4 AP”) for detection. As shown in FIG. 5B, the TNYL-RAW Fc fusion protein was able to bind EphB4 AP with substantial affinity. The results from one binding experiment to assay the binding affinity of TNYL-RAW Fc fusion protein for murine EphB4 AP and the affinity of ephrin-B2 Fc for murine EphB4 AP are shown in FIGS. 5C and 5D, respectively.

The half-life of the TNYL-RAW Fc protein appears to be longer than that of the synthetic peptide without Fc, since the TNYL-RAW Fc fusion protein was purified from medium in which the producing HEK293 cells were grown for 4 days.

The TNYL-RAW Fc protein at 2 μg/ml and clustered with anti-Fc antibodies did not stimulate EphB4 tyrosine phosphorylation in MDA-MB-231 breast cancer cells, unlike the positive control ephrin B2 Fc (FIG. 5E). EphB4 tyrosine phosphorylation was detected in EphB4 immunoprecipitates by immunoblotting with anti-phosphotyrosine antibodies (PTyr). MDA-MB-231 breast cancer cells were stimulated with 2 μg/ml TNYL-RAW Fc protein for 20 minutes prior to lysing the cells and obtaining EphB4 immunoprecipitates. Cells were also stimulated with 1 μg/ml ephrin-B2 Fc or human Fc as positive and negative controls, respectively.

### Example 8

### TNYL-RAW Peptide Linked to 40 kDa PEG

TNYL-RAW peptide (SEQ ID NO:39) synthesized and conjugated to biotin is coupled to PEG with a molecular weight of 40 kDa (TNYL-RAW-PEG40). TNYL-RAW-PEG40 is dialyzed prior to use in binding assays. Due to the size of the compound formed by the conjugation of TNYL-RAW to 40 kDa PEG, the biotin conjugated to TNYL-RAW normally used for detection in binding assays may not be exposed for detection. Therefore, a modified assay using AGP3 anti-PEG antibody is designed to measure the dissociation constant (K_{d}) and IC₅₀ of TNYL-RAW-PEG40.

Ni-NTA plates (Qiagen) are coated with a volume 100 μl/well of murine EphB4-Fc (R&D Systems, #446-B4-200) diluted in TBST/Ca (TBS, 0.01% Tween, 1 mM CaCl₂) to a final concentration of 1 μg/ml. After 1 hour at room temperature, the plates are washed 4 times with TBST/Ca. Subsequently, 100 μl of TNYL-RAW-PEG40 in TBST/Ca is added to each well at different concentrations. After a 1 hour incubation at room temperature, the plates are washed 4 times with TBST/Ca. AGP3 mouse anti-PEG IgM (from Academia Sinica, Taiwan) at a concentration of 1 μg/ml in TBST/Ca is added at a volume of 50 μl/well and allowed to incubate for another 1.5 hours at room temperature followed by 4 washes with TBST/Ca. Then, 100 μl of anti-mouse IgM-HRP (Serotec, #STAR86P)(dilution of 1:1000) is added to each well. After a 1 hour incubation at room temperature, the wells are washed 4 times with TBST/Ca, and bound complexes are detected with ABTS substrate (Sigma, #1888). The OD405 is measured with a plate reader. A filtered stock of ABTS substrate is prepared by adding 100 mg ABTS (Sigma, #1888) to 450 ml of 0.05 M citric acid (pH 4, filtered). Prior to use, 18 μl of 30% H₂O₂ is added to 10.5 ml ABTS stock solution.

For inhibition assays, plates are coated with a volume 100 μl/well of murine EphB4-Fc (R&D Systems, #446-B4-200) diluted in TBST/Ca (TBS, 0.01% Tween, 1 mM CaCl₂) to a final concentration of 1 μg/ml. After 1 hour at room temperature, the plates are washed 4 times with TBST/Ca. Subsequently, murine Ephrin B2 AP ligand in the presence or absence of various concentrations 100 μl of TNYL-RAW-PEG40 in TBST/Ca are added to the wells containing the immobilized EphB4-Fc. After a 1 hour incubation at room temperature, the plates are washed 4 times with TBST/Ca. The concentration of Ephrin B2 AP binding to the EphB4 receptor is measured at OD405 with a plate reader. In similar inhibition assays, plates are coated with a volume 100 μl/well of murine Ephrin B2-Fc diluted in TBST/Ca (TBS, 0.01% Tween, 1 mM CaCl₂). After 1 hour at room temperature, the plates are washed 4 times with TBST/Ca. Subsequently, murine EphB4 AP ligand in the presence or absence of various concentrations 100 μl of TNYL-RAW-PEG40 in TBST/Ca are added to wells containing immobilized Ephrin B2-Fc. After a 1 hour incubation at room temperature, the plates are washed 4 times with TBST/Ca. The concentration of EphB4 AP binding to the EphB4 receptor is measured at OD405 with a plate reader.

### Example 9

### EphB-Binding Multimeric Peptides as Targeting Agents

In addition to inhibiting the ability of Eph receptors to bind ephrins and elicit biological responses, EphB-binding multimeric peptides may selectively target other molecules, such as drugs or imaging probes, to EphB receptor-expressing cells. A stringent assay to determine the receptor-binding specificity of the peptides uses biotinylated peptides immobilized on streptavidin plates, which captures dimeric EphB Fc with high avidity (the apparent dissociation constants are in the low nM range). Biotinylated multimeric peptides are immobilized on streptavidin-coated plates and are used to capture EphB receptor Fc proteins. The binding affinity of the peptide is measured by immobilizing EphB4 Fc to protein A plates and measuring binding of biotinylated peptide using steptavidin-HRP to detect the bound biotinylated peptide. Bound receptor is detected using anti-Fc antibody coupled to alkaline phosphatase and is normalized to the value in the well with highest receptor binding. These experiments are designed to reveal binding of certain peptides to one or more EphB binding receptors.

### Example 10

### Binding Stability of Multimeric Peptides

The ability of the multimeric peptides to bind in a stable manner, which is important for both targeting and competitive inhibition, is tested in pull-down experiments. For peptide pull-down experiments, cells in 60 cm plates at 70% confluency or adult mouse brain tissue are solubilized in pull-down buffer (50 mM HEPES pH 7.5, 150 mM NaCl, 10% glycerol, 1% Triton-X100, 5 mM KCl and 1 mM EDTA). Three μg of biotinylated multimeric peptide are incubated for 45-90 min with 5 streptavidin agarose beads (Sigma), unbound multimeric peptide is washed away, and the beads are incubated with the cell lysates for 45-90 min. Proteins bound to the beads are separated by SDS-polyacrylamide gel electrophoresis and probed by immunoblotting with EphB1, EphB2, EphB3, EphB4, EphB5 or EphB6 antibodies. The EphB1 antibody (Santa Cruz) is detected with a secondary anti-goat IgG peroxidase-conjugated antibody (BioRad Laboratories). The EphB2 and EphB4 antibodies are affinity-purified polyclonal antibodies to GST fusion proteins containing approximately 100 amino acids from the carboxy-terminal tails of the EphB2 or EphB4 receptors (Noren, N. K. et al. 2004 PNAS USA 101:5583-5588; Holash, J. A. & Pasquale, E. B. 1995 Devel Biol 172:683-693) and are detected with a secondary anti-rabbit IgG peroxidase-conjugated antibody (Amersham Biosciences). Endogenous EphB receptors are isolated from lysates of mouse brain or cultured cells using the multimeric peptides immobilized on streptavidin beads. This study is used to detect the ability of multimeric peptides immobilized on streptavidin beads to bind EphB receptors from tissue and cell lines.

### Example 11

### Targeting Ability of Multimeric Peptides

To demonstrate the targeting ability of an EphB receptor-binding multimeric peptide, the multimeric peptide can be used to mediate binding of fluorescent streptavidin-coated quantum dot nanocrystals to cells expressing transfected as well as endogenous EphB receptors. For labeling of cells expressing transfected EphB receptor, COS cells in 6 cm plates are transfected with 3 μg of a plasmid encoding the EphB extracellular and transmembrane domains fused to enhanced green fluorescent protein (EGFP) (Ogawa, K. et al. 2000 Oncogene 19:6043-6052), or 3 μg of a vector encoding farnesylated EGFP (pEGFP-F) (BD Biosciences Clontech) as control, using SuperFect transfection reagent. The cells are plated on glass coverslips 1 day after transfection and labeled 2 days after transfection. For labeling experiments, 20 nM streptavidin-conjugated Qdot 655 quantum dots (Quantum Dot Corp.) are preincubated with 500 nM biotinylated multimeric peptide for 20 min on ice in quantum-dot-binding-buffer (1 mM CaCl₂, 2% BSA in PBS). The cells are incubated with quantum dots containing bound multimeric peptide, or quantum dots without multimeric peptide as a control, for 20 min at 4° C. and washed with ice cold 1 mM CaCl₂ in PBS. For labeling of cells endogenously expressing an EphB receptor, MCF-7 cells plated on glass coverslips coated with fibronectin (10 μg/ml) are incubated with 100 μM biotinylated multimeric peptide diluted in quantum-dot-binding-buffer for 20 min at 4° C. The cells are then washed with ice cold 1 mM CaCl₂ in PBS, followed by incubation with 20 nM streptavidin quantum dots for 20 min at 4° C. After labeling, the cells are fixed in 4% formaldehyde/4% sucrose for 10 min and permeabilized for 5 min with 0.05% Triton-X100 in PBS. The nuclei are counterstained with DAPI and the coverslips are mounted with ProLong Gold mounting media (Molecular Probes) onto glass slides and imaged and photographed under a fluorescence microscope. Green fluorescent protein marks the transfected cells.

MCF7 cells, which express endogenous EphB4, are labeled by quantum dots bound to a multimeric peptide but not by control quantum dots without multimeric peptide. MCF-7 human breast cancer cells, which endogenously express EphB4, are grown in Minimum Essential medium Eagle (MEM) (ATCC) with 10% fetal bovine serum, 0.01 mg/ml bovine insulin, and Pen/Strep. COS cells, which endogenously express EphB2, and 293 human embryonic kidney (HEK) cells are grown in Dulbecco's Modified Eagles medium (DME) with high glucose (Irvine Scientific) with 10% fetal calf serum, sodium pyruvate, and Pen/Strep. Ten cm plates of 293 HEK cells are transfected with 9 μg EphB4 cDNA in pcDNA3, and 1 μg of an enhanced green fluorescent protein plasmid (BD Biosciences Clontech) to verify transfection efficiency, using SuperFect transfection reagent (Qiagen). The cells are passaged 1 day after transfection and used for pull-down experiments 2 days after transfection. Nuclei of both transfected and untransfected cells are labeled with DAPI. The multimeric peptide is tested for its ability to bind to EphB4 after fixation of the cells with 4% formaldehyde.

### Example 12

### Administration of an EphB Receptor-Binding Multimeric Peptide in the Treatment of Cancer

A patient is identified by various diagnostic methods as being in need of treatment for colorectal cancer. A therapeutically effective amount of an EphB receptor binding compound, e.g., EphB receptor binding conjugate that is multimeric, is administered to the patient, who is monitored for amelioration of the colorectal cancer. Following such treatment, a reduction in the colorectal cancer in the patient is found.

### Example 13

### Administration of an EphB Receptor-Binding Multimeric Peptide in the Treatment of Cancer

A patient is identified by various diagnostic methods as being in need of treatment for a neoplastic disorder associated with abnormal angiogenesis. A therapeutically effective amount of an EphB receptor binding compound, e.g., EphB receptor binding conjugate that is multimeric, is administered to the patient prior to surgical and/or chemotherapeutic treatment to reduce neovascularization of the tumor thereby inducing shrinkage of the tumor. Following such treatment, the patient is monitored for a reduction in the size of the tumor.

### Example 14

### Administration of an EphB Receptor-Binding Multimeric Peptide in the Treatment of Chronic Pain

A patient complaining of neuropathic pain is administered a therapeutically effective amount of an EphB receptor binding compound, e.g., EphB receptor binding conjugate that is multimeric. A reduction in the level of the pain is observed and measured in the patient.

### Example 15

### Administration of an EphB Receptor-Binding Multimeric Peptide in the Treatment of Spinal Cord Injury

A patient with a spinal cord injury is administered a therapeutically effective amount of an EphB receptor binding compound, e.g., EphB receptor binding conjugate that is multimeric, that inhibits activity of the EphB receptor. Nerve regeneration at the site of injury is stimulated. Following such treatment, nerve regeneration at the site of stimulation is found in the patient.

### 7. EQUIVALENTS

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

We claim:

1.-67. (canceled)

68. An isolated EphB receptor binding compound comprising two or more peptides wherein each peptide selectively binds to an EphB receptor, and each of said two or more peptides has a length of between 5 to 50 amino acid residues.

69. The isolated EphB receptor binding compound according to claim 68 wherein the compound has a dissociation constant (Kd) of about 100 nM or less.

70. The isolated EphB receptor binding compound according to claim 68 wherein the compound inhibits the binding of the EphB receptor to an EphrinB ligand at an IC₅₀ of about 100 nM or less.

71. The isolated EphB receptor binding compound according to claim 68 wherein the compound further comprises a heterologous polypeptide, radioisotope, detectable label or peptidomimetic.

72. The isolated EphB receptor binding compound according to claim 68 wherein the compound further comprises an Fc region of human IgG or a fragment thereof.

73. The isolated EphB receptor binding compound according to claim 68 wherein the compound further comprises a polyethylene glycol (PEG) linker.

74. The isolated EphB receptor binding compound according to claim 68 wherein the compound further comprises at least one polypeptides selected from SEQ ID NOs:1-75.

75. The isolated EphB receptor binding compound according to claim 68, wherein the EphB receptor is a murine EphB receptor or a human EphB receptor.

76. The isolated EphB receptor binding compound according to claim 68 wherein the EphB receptor is EphB1, EphB2, EphB3, EphB4, EphB5, or EphB6.

77. A composition comprising the isolated EphB receptor binding compound according to claim 68 and a pharmaceutical carrier.

78. A method of using an isolated EphB receptor binding compound to bind an EphB receptor wherein the method comprises;
a. contacting a sample comprising an EphB receptor with the EphB receptor binding compound wherein the compound comprises two or more peptides each having a length of between 5 to 50 amino acid residues, to form a contacted sample; and
b. incubating the contacted sample for a sufficient amount of time for the EphB receptor binding compound to associate with the EphB receptor to form an incubated sample whereby the two or more peptides of the EphB receptor binding compound each selectively bind the EphB receptor.

79. The method according to claim 78, wherein the sample comprises cells expressing an EphB receptor, an EphB receptor attached to a solid support, an EphB receptor in an vitro system, an EphB receptor in an vivo system, a recombinant EphB receptor, an endogenous EphB receptor or variants thereof.

80. The method according to claim 79 wherein cells expressing an EphB receptor are depleted.

81. The method according to claim 79 wherein proliferation of cells expressing an EphB receptor is inhibited.

82. The method according to claim 79, wherein the EphB receptor in the sample is detected.

83. The method according to claim 82, wherein detecting an EphB receptor further comprises detecting aberrant expression of the EphB receptor.

84. The method according to claim 79, wherein an EphB receptor related disease is treated or prevented by the binding of the EphB receptor binding compound to the EphB receptor.

85. The method according to claim 84, wherein the EphB receptor binding compound is administered to a subject after removal of a tumor.

86. The method according to claim 84, wherein the EphB receptor related disease is selected from a neoplastic disease, a vascular disease, a neurological disorder, or cancer.

87. The method according to claim 84, further comprising determining the efficacy of the EphB receptor binding compound to treat an EphB receptor related disease.
